SEQUENCE LISTING (16 sequences)

SEQ ID NO: 1 - protein, 8 aa, synthetic construct
GFSLNRYY

SEQ ID NO: 2 - protein, 7 aa, synthetic construct
ISYGDTT

SEQ ID NO: 3 - protein, 15 aa, synthetic construct
ARADTGDNGYLGLQL

SEQ ID NO: 4 - protein, 6 aa, synthetic construct
QSIGSV

SEQ ID NO: 5 - skipped

SEQ ID NO: 6 - protein, 9 aa, synthetic construct
QYIPYGSSP

SEQ ID NO: 7 - protein, 121 aa, synthetic construct
QVQLVESGGRVVQPGRSLRLSCTASGFSLNRYYMLWVRQAPGKGLEWIGTISYGDTTYYASWAKGRFTISRDNSKNTLYLQMNSLRAEDTAVYYCARADTGDNGYLGLQLWGQGTLVTVSS

SEQ ID NO: 8 - protein, 107 aa, synthetic construct
DIQMTQSPSSVSASVGDRVTITCQASQSIGSVLAWYQQKPGKAPKLLISGVFDLASGVPSRFSGSGSGTDFTLTISSLQPEDFATYYCQYIPYGSSPFGGGTKVEIK

SEQ ID NO: 9 - protein, 107 aa, synthetic construct
DIQMTQSPSSVSASVGDRVTITCRASQSIGSVLAWYQQKPGKAPKLLIYGVFSLQSGVPSRFSGSGSGTDFTLTISSLQPEDFATYYCQYIPYGSSPFGGGTKVEIK

SEQ ID NO: 10 - protein, 451 aa, synthetic construct
QVQLVESGGRVVQPGRSLRLSCTASGFSLNRYYMLWVRQAPGKGLEWIGTISYGDTTYYASWAKGRFTISRDNSKNTLYLQMNSLRAEDTAVYYCARADTGDNGYLGLQLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 451 aa, synthetic construct
QVQLVESGGRVVQPGRSLRLSCTASGFSLNRYYMLWVRQAPGKGLEWIGTISYGDTTYYASWAKGRFTISRDNSKNTLYLQMNSLRAEDTAVYYCARADTGDNGYLGLQLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, synthetic construct
DIQMTQSPSSVSASVGDRVTITCQASQSIGSVLAWYQQKPGKAPKLLISGVFDLASGVPSRFSGSGSGTDFTLTISSLQPEDFATYYCQYIPYGSSPFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - RNA, 23 nt, synthetic construct
  source 1..23: mol_type = other RNA
tccatctccacgtagcccgtggg

SEQ ID NO: 14 - RNA, 23 nt, synthetic construct
  source 1..23: mol_type = other RNA
acttacgatggatatactgctgg

SEQ ID NO: 15 - protein, 211 aa, synthetic construct
ELVMTQTPASVEAAVGGTVTIKCQASQSIGSVLAWYQQKPGQRPKLLISGVFDLASGVPSRFKGSGSGTEFTLTISDLECADAATYYCQYIPYGSSPFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 16 - protein, 441 aa, synthetic construct
QSLEESGGRLVTPGTPLTLTCTASGFSLNRYYMLWVRQAPGEGLEWIGTISYGDTTYYASWAKGRFTISKTSTTVDLKMTSPTTEDTATYFCARADTGDNGYLGLQLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

(19) United States
(12) Patent Application Publication
(10) Pub. No.: US 2026/0199458 A1
(43) Pub. Date: Jul. 16, 2026

(54) METHODS OF TREATING CANCER USING ANTI-DDR1 ANTIBODIES AND IMMUNE CHECKPOINT INHIBITORS

(71) Applicant: Incendia Therapeutics, Inc., Cambridge, MA (US)

(72) Inventors: Laurent Audoly, Cambridge, MA (US); Laura Dillon, Cambridge, MA (US); Thomas Schuerpf, Cambridge, MA (US); Joseph Eder, Cambridge, MA (US); Guy Travis Clifton, Cambridge, MA (US); Yan Qin, Cambridge, MA (US)

(21) Appl. No.: 19/130,509

(22) PCT Filed: Nov. 15, 2023
(86) PCT No.: PCT/US2023/079907

§ 371 Date: May 15, 2025

Related U.S. Application Data
(60) Provisional application No. 63/383,954, filed on Nov. 16, 2022.
(60) Provisional application No. 63/519,935, filed on Aug. 16, 2023.

(51) Int. Cl. A61K 39/395; A61K 39/00; A61P 35/00

(52) U.S. Cl. CPC A61K 39/3955; A61P 35/00; A61K 2039/507; A61K 2039/54; A61K 2039/545

(57) ABSTRACT

Provided are methods and dosage regimens for treating cancer using an antibody that specifically binds to human DDR1 and an immune checkpoint inhibitor.

### REFERENCE TO RELATED APPLICATIONS

This application claims priority to U.S. Provisional Application Ser. No. 63/383,954, filed Nov. 16, 2022, and 63/519,935, filed Aug. 16, 2023. The contents of each of these related applications are hereby incorporated by reference in its entirety.

### REFERENCE TO SEQUENCE LISTING

The present application is being filed along with a Sequence Listing in electronic format. The Sequence Listing is provided as a file entitled INCEN011WOseqlist.xml, created Nov. 15, 2023, which is 16,422 bytes in size. The information in the electronic format of the Sequence Listing is incorporated herein by reference in its entirety.

### FIELD

The instant disclosure relates to methods of treating discoidin domain receptor tyrosine kinase 1 (DDR1) related disorders through administering an anti-DDR1 antibody in combination with an immune checkpoint inhibitor.

### BACKGROUND

Cancer immune exclusion is a cancer phenotype characterized by a spatial imbalance with more immunologic cells in proximity to the tumor but fewer immune cells in physical contact with tumor cells and is prevalent in a high proportion of tumors. Altering immune-excluded tumors to become immune-accessible is an active area of oncology research.

Accordingly, there remains an unmet medical need for treatment options for immune-excluded cancers.

### SUMMARY

Discoidin domain receptor tyrosine kinase 1 (DDR1) is a receptor tyrosine kinase that is widely expressed in normal and transformed epithelial cells and is activated by various types of collagens. DDR1 autophosphorylation is achieved by all collagens so far tested (type I to type VI). DDR1 is predominantly expressed in normal epithelial cells, and it is aberrantly over-expressed in a variety of human cancers. Its expression is associated with tumor progression, including breast, lung, ovary, liver, gastric cancer, and glioma. An elevated DDR1 signature is associated with more immunologically cold tumors and decreased responsiveness to immune checkpoint inhibitor therapy in non-small cell lung cancer.

9H-1 is a first-in-class, humanized monoclonal antibody targeting human DDR1 and is designed to disrupt the tumor stroma and allow a subject's own immune cells to infiltrate the tumor to destroy the tumor. Robust single agent activity has been observed across multiple preclinical tumor models. Accordingly, 9H-1 may provide an effective treatment for cancer, particularly immune-excluded cancer. Moreover, the use of 9H-1 in combination with an immune checkpoint inhibitor may lead to further improvements in anti-tumor capabilities over either monotherapy alone.

Embodiments of the present disclosure are directed to methods for reducing immune exclusion of a tumor and/or methods for reducing tumor burden in a subject in need thereof with an antibody that specifically binds to human discoidin domain receptor tyrosine kinase 1 (DDR1) and an immune checkpoint inhibitor. Also provided herein are embodiments of particular dosage regimens for administering an antibody that specifically binds to human DDR1.

Provided herein are the following numbered embodiments:
1. A method of reducing immune exclusion of a tumor in a subject in need thereof, the method comprising administering to the subject an anti-DDR1 antibody that specifically binds to human DDR1 and an immune checkpoint inhibitor, wherein the anti-DDR1 antibody is administered at a dose of 5 mg to 2000 mg.
2. A method of reducing tumor burden in a subject in need thereof, the method comprising administering to the subject an anti-DDR1 antibody that specifically binds to human DDR1 and an immune checkpoint inhibitor, wherein the anti-DDR1 antibody is administered at a dose of 5 mg to 2000 mg.
3. The method of embodiment 1 or 2, wherein the anti-DDR1 antibody is administered at a dose of about 8 mg to about 1600 mg.
4. The method of embodiment 1 or 2, wherein the anti-DDR1 antibody is administered at a dose of about 8 mg to about 800 mg.
5. The method of any one of embodiments 1-4, wherein the anti-DDR1 antibody is administered at a dose of about 8 mg, about 24 mg, about 80 mg, about 240 mg, about 400 mg, about 800 mg, or about 1600 mg.
6. The method of any one of embodiments 1-5, wherein the anti-DDR1 antibody is administered at a dose of 8 mg, 24 mg, 80 mg, 240 mg, 400 mg, 800 mg, or 1600 mg.
7. The method of any one of embodiments 1-6, wherein the anti-DDR1 antibody is administered intravenously.
8 The method of any one of embodiments 1-7, wherein the anti-DDR1 antibody is administered via intravenous infusion over 60 minutes.
9 The method of any one of embodiments 1-7, wherein the anti-DDR1 antibody is administered via intravenous infusion over 30 minutes.
10. The method of any one of embodiments 1-9, wherein the anti-DDR1 antibody is administered once weekly.
11. The method of any one of embodiments 1-9, wherein the anti-DDR1 antibody is administered once every 2 weeks.
12. The method of any one of embodiments 1-9, wherein the anti-DDR1 antibody is administered once every 3 weeks.
13. The method of any one of embodiments 1-9, wherein the anti-DDR1 antibody is administered once every 4 weeks.
14. The method of any one of embodiments 1-9, wherein the anti-DDR1 antibody is administered once every 8 weeks.
15. The method of any one of embodiments 1-9, wherein the anti-DDR1 antibody is administered intravenously at a dose of 8 mg once every 3 weeks.
16. The method of any one of embodiments 1-9, wherein the anti-DDR1 antibody is administered intravenously at a dose of 24 mg once every 3 weeks.
17. The method of any one of embodiments 1-9, wherein the anti-DDR1 antibody is administered intravenously at a dose of 80 mg once every 3 weeks.
18. The method of any one of embodiments 1-9, wherein the anti-DDR1 antibody is administered intravenously at a dose of 240 mg once every 3 weeks.
19. The method of any one of embodiments 1-9, wherein the anti-DDR1 antibody is administered intravenously at a dose of 400 mg once every 3 weeks.
20. The method of any one of embodiments 1-9, wherein the anti-DDR1 antibody is administered intravenously at a dose of 800 mg once every 3 weeks.
21. The method of any one of embodiments 1-9, wherein the anti-DDR1 antibody is administered intravenously at a dose of 1600 mg once every 3 weeks.
22. The method of any one of embodiments 1-21, wherein the immune checkpoint inhibitor comprises a PD-1 or PD-L1 antagonist.
23. The method of any one of embodiments 1-22, wherein the PD-1 or PD-L1 antagonist is administered at a dose of 100 mg to 2000 mg.
24. The method of any one of embodiments 1-22, wherein the PD-1 or PD-L1 antagonist is administered at a dose of 200 mg, 240 mg, 350 mg, 360 mg, 400 mg, 480 mg, 500 mg, 840 mg, 1000 mg, 1200 mg, 1500 mg, or 1680 mg.
25. The method of any one of embodiments 1-22, wherein the PD-1 or PD-L1 antagonist is administered at a dose of 0.5 mg/kg to 30 mg/kg.
26. The method of any one of embodiments 1-22, wherein the PD-1 or PD-L1 antagonist is administered at a dose of 1 mg/kg, 2 mg/kg, 3 mg/kg, 10 mg/kg, or 20 mg/kg.
27. The method of any one of embodiments 1-26, wherein the PD-1 or PD-L1 antagonist is administered intravenously.
28. The method of any one of embodiments 1-27, wherein the PD-1 or PD-L1 antagonist is administered once weekly.
29. The method of any one of embodiments 1-27, wherein the PD-1 or PD-L1 antagonist is administered once every 2 weeks.
30. The method of any one of embodiments 1-27, wherein the PD-1 or PD-L1 antagonist is administered once every 3 weeks.
31. The method of any one of embodiments 1-27, wherein the PD-1 or PD-L1 antagonist is administered once every 4 weeks.
32. The method of any one of embodiments 1-27, wherein the PD-1 or PD-L1 antagonist is administered once every 6 weeks.
33. The method of any one of embodiments 1-27, wherein the PD-1 or PD-L1 antagonist is administered once every 8 weeks.
34. The method of any one of the preceding embodiments, wherein the subject has cancer.
35. The method of embodiment 34, wherein the administration of the anti-DDR1 antibody and the PD-1 or PD-L1 antagonist treats the cancer in the subject.
36. The method of embodiment 34, wherein the PD-1 antagonist is an anti-PD-1 antibody that specifically binds to human PD-1.
37. The method of embodiment 34, wherein the PD-L1 antagonist is an anti-PD-L1 antibody that specifically binds to human PD-L1.
38. The method of embodiment 36, wherein the anti-PD-1 antibody is pembrolizumab, nivolumab, dostarlimab, cemiplimab, sintilimab, penpulimab, tislelizumab, toripalimab, or retifanlimab.
39. The method of embodiment 37, wherein the anti-PD-L1 antibody is avelumab, atezolizumab, or durvalumab.
40. The method of any one of embodiments 1-36, wherein the PD-1 antagonist is pembrolizumab.
41. The method of embodiment 40, wherein pembrolizumab is administered at a dose of 400 mg once every 6 weeks.
42. The method of embodiment 40, wherein pembrolizumab is administered at a dose of 200 mg once every 3 weeks.
43. The method of embodiment 40, wherein pembrolizumab is administered at a dose of 2 mg/kg once every 3 weeks.
44. The method of any one of embodiments 1-36, wherein the PD-1 antagonist is nivolumab.
45. The method of embodiment 44, wherein nivolumab is administered at a dose of 240 mg once every 2 weeks.
46. The method of embodiment 44, wherein nivolumab is administered at a dose of 360 mg once every 3 weeks.
47. The method of embodiment 44, wherein nivolumab is administered at a dose of 480 mg once every 4 weeks.
48. The method of embodiment 44, wherein nivolumab is administered at a dose of 3 mg/kg once every 2 weeks.
49. The method of embodiment 44, wherein nivolumab is administered at a dose of 3 mg/kg once every 3 weeks.
50. The method of any one of embodiments 1-36, wherein the PD-1 antagonist is cemiplimab.
51. The method of embodiment 50, wherein cemiplimab is administered at a dose of 350 mg once every 3 weeks.
52. The method of any one of embodiments 1-36, wherein the PD-1 antagonist is dostarlimab.
53. The method of embodiment 52, wherein dostarlimab is administered at a dose of 500 mg once every 3 weeks.
54. The method of embodiment 52, wherein dostarlimab is administered at a dose of 1000 mg once every 6 weeks.
55. The method of any one of embodiments 1-35, or 37, wherein the PD-L1 antagonist is atezolizumab.
56. The method of embodiment 55, wherein atezolizumab is administered at a dose of 840 mg once every 2 weeks.
57. The method of embodiment 55, wherein atezolizumab is administered at a dose of 1200 mg once every 3 weeks.
58. The method of embodiment 55, wherein atezolizumab is administered at a dose of 1680 mg once every 4 weeks.
59. The method of any one of embodiments 1-35, or 37, wherein the PD-L1 antagonist is durvalumab.
60. The method of embodiment 59, wherein durvalumab is administered at a dose of 10 mg/kg once every 2 weeks.
61. The method of embodiment 59, wherein durvalumab is administered at a dose of 20 mg/kg once every 3 weeks.
62. The method of embodiment 59, wherein durvalumab is administered at a dose of 1500 mg once every 3 weeks.
63. The method of any one of the preceding embodiments, wherein the cancer expresses DDR1.
64. The method of any one of the preceding embodiments, wherein the cancer is a solid cancer.
65. The method of any one of the preceding embodiments, wherein the cancer is a locally advanced or metastatic solid cancer.
66. The method of any one of the preceding embodiments, wherein the cancer is unresectable.
67. The method of any one of the preceding embodiments, wherein the cancer is refractory to immunotherapy.
68. The method of embodiment 67, wherein the immunotherapy is an antagonist anti-PD-1 antibody, an antagonist anti-PD-L1 antibody, an antagonist anti-PD-L2 antibody, an antagonist anti-PD-1/anti-PD-L1 antibody bispecific antibody, an antagonist anti-CTLA-4 antibody, an antagonist anti-BTLA antibody, an antagonist anti-TREMR antibody, an antagonist anti-TIGIT antibody, an antagonist anti-VISTA antibody, an antagonist anti-TIM-3 antibody, an antagonist anti-LAG-3 antibody, an antagonist anti-CEACAM1 antibody, an agonist anti-GITR antibody, an agonist anti-OX40 antibody, and an agonist anti-CD137 antibody, an agonist anti-DR3 antibody, an agonist anti-TNFSF14 antibody, an agonist anti-CD27 antibody, an agonist anti-ICOS antibody, or an agonist anti-CD28 antibody.
69. The method of embodiment 67, wherein the immunotherapy is an antagonist anti-PD-L1 antibody.
70. The method of any one of the preceding embodiments, wherein the cancer is not sarcoma, hepatocellular carcinoma, or glioma.
71. The method of any one of the preceding embodiments, wherein the cancer is pancreatic cancer, lung cancer, small cell lung cancer, non-small cell lung cancer, colorectal cancer, head and neck cancer, stomach (gastric) cancer, ovarian cancer, breast cancer, kidney cancer, prostate cancer, cervical cancer, brain cancer, skin cancer, melanoma, cholangiocarcinoma, or bone cancer.
72. The method of any one of the preceding embodiments, wherein the cancer is colorectal cancer, ovarian cancer, or non-small cell lung cancer.
73. The method of any one of the preceding embodiments, wherein the subject is not a candidate for standard of care treatment.
74. The method of any one of the preceding embodiments, wherein the cancer is refractory to a standard of care treatment.
75. The method of embodiment 74, wherein the standard of care treatment is chemotherapy or radiation.
76. The method of any one of embodiments 1-75, wherein administration of the anti-DDR1 antibody and the PD-1 or PD-L1 antagonist reduces tumor size in the subject.
77. The method of any one of the preceding embodiments, wherein before administration of the anti-DDR1 antibody and the PD-1 or PD-L1 antagonist the subject:
a) has confirmed metastatic or advanced, unresectable cancer, measurable disease per Response Evaluation Criteria in Solid Tumors (RECIST) v1.1;
b) has a pathologically documented advanced, unresectable, or metastatic cancer that is refractory to or intolerable to standard treatment known to confer benefit or for which no standard treatment is available;
c) has an Eastern Cooperative Oncology Group performance status (PS) 0-1;
d) has a predicted life expectancy of ≥3 months;
e) has one or more of:

$$i ) calculated creatinine clearance ( CrCL ) \geq 50 mL / min by Crockcroft - Gault fomulation calculation ;$$

$$ii ) total bilirubin \leq 1.5 ;$$

$$iii ) AST and ALT \leq 2.5 \times ULN ;$$

$$iv ) hemoglobin \geq 9. g / dL ;$$

$$v ) platelets \geq 100 \times {10}^{9} cells / L ; or$$

$$vi ) absolute neutrophil count \geq 1.5 \times {10}^{9} cells / L ;$$

f) has a Corrected QT interval (QTc)≤470 ms (as calculated by the Fridericia correction formula); and/or
g) is not receiving other cancer therapy.
78. The method of any one of the preceding embodiments, wherein before administration of the anti-DDR1 antibody and the PD-1 or PD-L1 antagonist the subject:
a) has not received prior treatment with systemic agents comprising radio-immunoconjugates, antibody-drug conjugates, immune/cytokines or monoclonal antibodies within 28 days or five half-lives of the drug, whichever is shorter;
b) does not have ongoing toxicity from prior therapy;
c) has not undergone a major surgery<3 months prior to administration of the anti-DDR1 antibody;
d) has not received radiation therapy<28 days prior to administration of the anti-DDR1 antibody;
e) has not undergone organ transplantation, allogeneic stem-cell transplantation, or autologous stem-cell transplantation;
f) has not received a diagnosis of primary or acquired immunodeficiency;
g) has not received treatment with systemic steroids or any other form of immunosuppressive therapy within 14 days prior to administration of the anti-DDR1 antibody;
h) does not have central nervous system (CNS) tumor involvement not definitively treated with surgery or radiation that is active;
i) does not have active autoimmune disease requiring immunosuppressive therapy or a history of such disease;
j) does not have clinical symptoms of CNS metastases within 28 days prior to administration of the anti-DDR1 antibody; and/or
k) does not have leptomeningeal carcinomatosis.
79. The method of any one of the preceding embodiments, wherein the anti-DDR1 antibody comprises: a heavy chain variable region (VH) comprising the CDRH1, CDRH2, and CDRH3 amino acid sequences of the VH amino acid sequence set forth in SEQ ID NO: 7, or a variant thereof comprising 1-5 amino acid changes in any one of the CDRH1, CDRH2, or CDRH3 amino acid sequences; and/or a light chain variable region (VL) comprising the CDRL1, CDRL2, and CDRL3 amino acid sequences of the VL amino acid sequence set forth in SEQ ID NO: 8 or 9, or a variant thereof comprising 1-5 amino acid changes in any one of the CDRL1, CDRL2, or CDRL3 amino acid sequences.
80. The method of embodiment 79, wherein:
   (a) the VH comprises the CDRH1, CDRH2, and CDRH3 amino acid sequences, respectively, of:
SEQ ID NO: 1, or a variant thereof comprising 1-5 amino acid changes,
SEQ ID NO: 2, or a variant thereof comprising 1-5 amino acid changes, and
SEQ ID NO: 3, or a variant thereof comprising 1-5 amino acid changes; and/or
   (b) the VL comprises the CDRL1, CDRL2, and CDRL3 amino acid sequences, respectively, of:
SEQ ID NO: 4, or a variant thereof comprising 1-5 amino acid changes,
SEQ ID NO: 5, or a variant thereof comprising 1-5 amino acid changes, and
SEQ ID NO: 6, or a variant thereof comprising 1-5 amino acid changes.
81. The method of embodiment 79 or 80, wherein the anti-DDR1 antibody that specifically binds to human DDR1 comprises the CDRH1, CDRH2, CDRH3, CDRL1, CDRL2, and CDRL3 amino acid sequences set forth in SEQ ID NOs: 1, 2, 3, 4, 5, and 6, respectively.
82. The method of any one of embodiments 79-81, wherein the anti-DDR1 antibody that specifically binds to human DDR1 comprises: a VH comprising an amino acid sequence which is at least 75%, 80%, 85%, 90%, 95%, 96%, 97%, 98%, 99%, or 100% identical to the amino acid sequence set forth in SEQ ID NO: 7; and/or a VL comprising an amino acid sequence which is at least 75%, 80%, 85%, 90%, 95%, 96%, 97%, 98%, 99%, or 100% identical to the amino acid sequence set forth in SEQ ID NO: 8 or 9.
83. The method of embodiment 82, wherein the anti-DDR1 antibody comprises a VH comprising the amino acid sequence set forth in SEQ ID NO: 7 and a VL comprising the amino acid sequence set forth in SEQ ID NO: 8.
84. The method of embodiment 82, wherein the anti-DDR1 antibody comprises a VH comprising the amino acid sequence set forth in SEQ ID NO: 7 and a VL comprising the amino acid sequence set forth in SEQ ID NO: 9.
85. The method of any one of embodiments 79-84, wherein the anti-DDR1 antibody comprises a heavy chain comprising an amino acid sequence which is at least 75%, 80%, 85%, 90%, 95%, 96%, 97%, 98%, 99%, or 100% identical to the amino acid sequence set forth in SEQ ID NO: 10 or 11 and/or a light chain comprising an amino acid sequence which is at least 75%, 80%, 85%, 90%, 95%, 96%, 97%, 98%, 99%, or 100% identical to the amino acid sequence set forth in SEQ ID NO: 12.
86. The method of any one of the preceding embodiments, wherein the anti-DDR1 antibody comprises a heavy chain comprising the amino acid sequence set forth in SEQ ID NO: 10 and a light chain comprising the amino acid sequence set forth in SEQ ID NO: 12.
87. The method of any one of embodiments 1-85, wherein the anti-DDR1 antibody comprises a heavy chain comprising the amino acid sequence set forth in SEQ ID NO: 10, without the terminal lysine, and a light chain comprising the amino acid sequence set forth in SEQ ID NO: 12.
88. The method of any one of embodiments 1-85, wherein the anti-DDR1 antibody comprises a heavy chain comprising the amino acid sequence set forth in SEQ ID NO: 11 and a light chain comprising the amino acid sequence set forth in SEQ ID NO: 12.
89. The method of any one of embodiments 1-85, wherein the anti-DDR1 antibody comprises a heavy chain comprising the amino acid sequence set forth in SEQ ID NO: 11, without the terminal lysine, and a light chain comprising the amino acid sequence set forth in SEQ ID NO: 12.
90. The method of any one of the preceding embodiments, wherein the anti-DDR1 is administered to the subject before the PD-1 or PD-L1 antagonist.
91. The method of any one of the preceding embodiments, wherein the anti-DDR1 antibody is administered to the subject after the PD-1 or PD-L1 antagonist.
92. The method of any one of the preceding embodiments, wherein the anti-DDR1 antibody is administered to the subject at the same time as the PD-1 or PD-L1 antagonist.
93. The method of any one of the preceding embodiments, wherein administration of the antibody and the immune checkpoint inhibitor prevents further growth of tumor size in the subject.
94. The method of any one of the preceding embodiments, wherein administration of the antibody and the immune checkpoint inhibitor achieves at least stable disease in the subject.
95. An anti-DDR1 antibody and a PD-1 or PD-L1 antagonist for use in the treatment of cancer, wherein the treatment is performed according to the method of any one of the previous embodiments.
96. An anti-DDR1 antibody and a PD-1 or PD-L1 antagonist for use in the manufacture of a medicament for the treatment of cancer, wherein the treatment is performed according to the method of any one of the previous embodiments.
97. Use of an anti-DDR1 antibody and a PD-1 or PD-L1 antagonist for the treatment of cancer, wherein the treatment is performed according to the method of any one of the previous embodiments.
98. A therapeutic combination comprising an antibody that specifically binds to human DDR1 and a PD-1 or PD-L1 antagonist.
99. A combination comprising an anti-DDR1 antibody and a PD-1 or PD-L1 antagonist for use in the treatment of cancer, wherein the treatment is performed according to the method of any one of the previous embodiments.

### BRIEF DESCRIPTION OF THE DRAWINGS

FIG. 1 is a bar graph of an embodiment showing 9H-1 binding at various concentrations in a flow cytometry assay.

FIG. 2 is a graph of an embodiment showing tumor volume in mice treated with 9H-1, 9H-1-LALAPG, or an IgG control.

FIG. 3A-D are graphs of an embodiment showing the concentrations of 9H-1 individual cynomolgus monkeys in all dose groups (FIG. 3A), the low dose group alone (FIG. 3B), the intermediate dose group (FIG. 3C), and the high dose group alone (FIG. 3D).

FIG. 4 is a graph of an embodiment showing simulated 9H-1 concentrations at a dosage interval of every 2 weeks.

FIG. 5 is a graph of an embodiment showing simulated 9H-1 concentrations at a dosage interval of every 3 weeks.

FIG. 6 is a graph of an embodiment showing the number of CD3+ T cells as a percentage of the total number of cells in the margin and core of solid tumors in a mouse tumor model. Mice were treated with vehicle (Control), a rabbit monoclonal antibody version of 9H-1 (αDDR1), pembrolizumab (αPD-1), or a combination of the rabbit monoclonal antibody version of 9H-1 and pembrolizumab (Combo).

FIG. 7A-7B depict the number of GZMB⁺ (left) and IFNγ⁺ (right) cells as a percentage of the total number of CD8⁺ T cells (FIG. 7A) and tumor volume over time (FIG. 7B) in a mouse solid tumor model according to some non-limiting embodiments. Mice were treated with vehicle (Control), a rabbit monoclonal antibody version of 9H-1 (αDDR1), mouse anti-PD-1 antibody (αPD-1), or a combination of the rabbit monoclonal antibody version of 9H-1 and mouse anti-PD-1 antibody (Combo).

FIG. 8 depicts a schematic model of the proposed mechanism by which anti-DDR1 enhances the anti-tumor effects of immune checkpoint inhibitors according to some non-limiting embodiments.

FIG. 9A-9D are graphs of an embodiment showing changes in individual tumor volume (mm³) (FIGS. 9A and 9B), average tumor volume (mm³) (FIG. 9C), and probability of survival (FIG. 9D) over time in B16F10 DDR1−/− tumors in C57BL/6 mice over 23 days post-inoculation.

FIG. 10A-10C are graphs of an embodiment showing changes in individual tumor volume (mm³) (FIG. 10A); average tumor volume (mm³) (FIG. 10B); and body weight (g) (FIG. 10C) in LLC1 DDR1−/− tumors in C57BL/6 mice over 26 days post-inoculation.

FIG. 11A-11C are graphs of an embodiment showing changes in individual tumor volume (mm³) (FIG. 11A); average tumor volume (mm³) (FIG. 11B); and body weight (g) (FIG. 11C) in E0771 DDR−/− tumors in C57BL/6 mice over 31 days post-inoculation.

FIG. 12A-12C are graphs of an embodiment showing changes in individual tumor volume (mm³) (FIG. 12A); average tumor volume (mm³) (FIG. 12B); and body weight (g) (FIG. 12C) in RENCA DDR1−/− tumors in Balb/c mice over 31 days post-inoculation.

FIG. 13A-13D are graphs of an embodiment showing changes in individual tumor volume (mm³) (FIG. 13A); average tumor volume (mm³) (FIG. 13B); body weight (g) (FIG. 13C); and average body weight (g) (FIG. 13D) in 4T1 DDR1−/− tumors in Balb/c mice over 38 days post-inoculation.

FIG. 14A-14C are graphs of an embodiment showing changes in individual tumor volume (mm³) (FIG. 14A); average tumor volume (mm³) (FIG. 14B); and average body weight (g) (FIG. 14C) in EMT6 DDR1−/− tumors in Balb/c mice over 31 days post-inoculation.

FIG. 15A-15D are graphs of an embodiment showing changes in individual tumor volume (mm³) (FIG. 15A); average tumor volume (mm³) (FIG. 15B); tumor volume (mm³) (FIG. 15C); and average body weight (g) (FIG. 15D) in CT26 DDR1−/− tumors in Balb/c mice over 38 days post-inoculation.

FIG. 16A-16D are graphs of an embodiment showing changes in individual tumor volume (mm³) (FIG. 16A); average tumor volume (mm³) (FIG. 16B); and average body weight (g) (FIG. 16C) and probability of survival (FIG. 16D) in MBT-2 DDR1−/− tumors in C3H/HeN mice over 11 days post-inoculation.

FIG. 17 shows the study design of an embodiment for tumor kinetics study with CT26 (wild type (WT) and knock out (KO)) cell lines in BALB/c mice.

FIG. 18A-18B are graphs of an embodiment showing changes in tumor volume (mm³) (FIG. 18A) and changes in % body weight (FIG. 18B) in CT26 DDR1 WT+CT26 DDR1 KO (n=10) over 42 days post-inoculation.

FIG. 19A-19B are graphs of an embodiment showing changes in individual tumor volumes (mm³) for CT26 WT cell lines (FIG. 19A) and CT26 KO cell lines (FIG. 19B) over 42 days post-inoculation.

FIG. 20A-20C show flow cytometry analysis plot of an embodiment of a CT26 parental line using an isotype antibody control (FIG. 20A) and using an anti-DDR1 antibody (FIG. 20B); and flow cytometry analysis plot of CT26 DDR1r line using an anti-DDR1 antibody post flow sorting (FIG. 20C).

### DETAILED DESCRIPTION

Embodiments of the present disclosure are directed to methods for treating cancer with an antibody that specifically binds to discoidin domain receptor tyrosine kinase 1 (DDR1), including human DDR1 (i.e., “specifically binds to human DDR1”), and an immune checkpoint inhibitor. In some embodiments, the immune checkpoint inhibitor is a PD-1 antagonist or a PD-L1 antagonist. In some embodiments, the immune checkpoint inhibitor comprises a PVRIG antagonist. In some embodiments, the PVRIG antagonist is an anti-PVRIG antibody that specifically binds to human PVRIG. Also provided herein are embodiments of particular dosage regimens for administering an antibody that specifically binds to human DDR1, as well as dosing regimens for combination therapies including PD-1 antagonists and PD-L1 antagonists. In some embodiments, the dosing regimens provided in the present disclosure provide serum exposure with anti-DDR1 antibody in human subjects above those necessary to inhibit DDR1 function thereby potentiating the therapeutic effect of co-administered immune checkpoint inhibitors.

### Terms

Unless defined otherwise, all technical and scientific terms used herein have the same meaning as is commonly understood by one of skill in the art to which the claimed subject matter belongs when read in light of the current disclosure. It is to be understood that the foregoing general description and the following detailed description are exemplary and explanatory only and are not restrictive of any subject matter claimed.

As used herein, the terms “antibody” and “antibodies,” have their plain and ordinary meaning as understood in light of the specification, and, unless specified otherwise, and include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA, or IgY), any class (e.g., IgG1, IgG2, IgG3, IgG4, IgA1, or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof. In an embodiment, the antibody is a humanized monoclonal antibody. In an embodiment, the antibody is a human monoclonal antibody.

As used herein, the term “CDR” or “complementarity determining region” has its plain and ordinary meaning as understood in light of the specification, and, unless specified otherwise, and means the noncontiguous antigen combining sites found within the variable regions of heavy and light chain polypeptides. These particular regions have been described by, for example, Kabat et al., J. Biol. Chem. 252, 6609-6616 (1977) and Kabat et al., “Sequences of Proteins of Immunological Interest.” (1991), by Chothia et al., J. Mol. Biol. 196:901-917 (1987), and by MacCallum et al., J. Mol. Biol. 262:732-745 (1996), all of which are herein incorporated by reference in their entireties, where the definitions include overlapping or subsets of amino acid residues when compared against each other (see Table 1 below). In certain embodiments, the term “CDR” is a CDR as defined by MacCallum et al., J. Mol. Biol. 262:732-745 (1996) and Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., “Sequences of Proteins of Immunological Interest.” (1991). In certain embodiments, heavy chain CDRs and light chain CDRs of an antibody are defined using different conventions. In certain embodiments, heavy chain CDRs and/or light chain CDRs are defined by performing structural analysis of an antibody and identifying residues in the variable region(s) predicted to make contact with an epitope region of a target molecule (e.g., human DDR1). CDRH1, CDRH2, and CDRH3 denote the heavy chain CDRs, and CDRL1, CDRL2, and CDRL3 denote the light chain CDRs.

TABLE 1
CDR definitions.

CDR Definitions

| | Kabat | Chothia | MacCallum |
|---|---|---|---|
| VH CDR1 | 31-35 | 26-32 | 30-35 |
| VH CDR2 | 50-65 | 53-55 | 47-58 |
| VH CDR3 | 95-102 | 96-101 | 93-101 |
| VL CDR1 | 24-34 | 26-32 | 30-36 |
| VL CDR2 | 50-56 | 50-52 | 46-55 |
| VL CDR3 | 89-97 | 91-96 | 89-96 |

As used herein, the terms “variable region” and “variable domain” have their plain and ordinary meaning as understood in light of the specification, and, unless specified otherwise, and are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable region are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In an embodiment, the variable region is a primate (e.g., non-human primate) variable region. In an embodiment, the variable region comprises rodent or murine CDRs and primate (e.g., non-human primate) framework regions (FRs).

As used herein, the terms “VH” and “VL” have their plain and ordinary meaning as understood in light of the specification, and, unless specified otherwise, and refer to antibody heavy and light chain variable regions, respectively, as described in Kabat et al., (1991) “Sequences of Proteins of Immunological Interest”, which is herein incorporated by reference in its entirety.

As used herein, the term “constant region” has its plain and ordinary meaning as understood in light of the specification, and, unless specified otherwise, and is common in the art. The constant region is an antibody portion, e.g., a carboxyl terminal portion of a light and/or heavy chain, which is not directly involved in binding of an antibody to antigen, but which can exhibit various effector functions, such as interaction with an Fc receptor (e.g., Fc gamma receptor).

As used herein, the term “heavy chain” has its plain and ordinary meaning as understood in light of the specification, and, unless specified otherwise, and when used in reference to an antibody can refer to any distinct type, e.g., alpha (a), delta (8), epsilon (¿), gamma (Y), and mu (u), based on the amino acid sequence of the constant region, which give rise to IgA, IgD, IgE, IgG, and IgM classes of antibodies, respectively, including subclasses of IgG, e.g., IgG1, IgG2, IgG3, and IgG4.

As used herein, the term “light chain” has its plain and ordinary meaning as understood in light of the specification, and, unless specified otherwise, and when used in reference to an antibody can refer to any distinct type, e.g., kappa (κ) or lambda (%), based on the amino acid sequence of the constant region. Light chain amino acid sequences are well known in the art. In an embodiment, the light chain is a human light chain.

As used herein, the terms “specifically binds,” “specifically recognizes,” “immunospecifically binds,” and “immunospecifically recognizes” have their plain and ordinary meaning as understood in light of the specification, and, unless specified otherwise, and are analogous terms in the context of antibodies and refer to molecules that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, ID), or other assays known in the art. In an embodiment, molecules that specifically bind to an antigen bind to the antigen with a KA that is at least 2 logs (e.g., factors of 10), 2.5 logs, 3 logs, 4 logs or greater than the KA when the molecules bind non-specifically to another antigen. Unless expressly indicated otherwise, and antibody or fragment thereof that is designated as “specifically binding” to an antigen, where the antigen is identified as being from a particular species (e.g., human) may bind to the same antigen of another species with the same or similar affinity. For example, an antibody or fragment thereof which “specifically binds to human DDR1” may bind with similar affinity to mouse DDR1.

As used herein, the term “EU numbering system” has its plain and ordinary meaning as understood in light of the specification, and, unless specified otherwise, and refers to the EU numbering convention for the constant regions of an antibody, as described in Edelman G.M. et al., Proc. Natl. Acad. USA, 63, 78-85 (1969) and Kabat et al., “Sequences of Proteins of Immunological Interest”, 1991, each of which is herein incorporated by reference in its entirety.

As used herein, the term “subject” includes any human or non-human animal. In an embodiment, the subject is a human. In some embodiments, “subject” and “patient” are used interchangeably.

As used herein, the term “cancer” has its plain and ordinary meaning as understood in light of the specification, and, unless specified otherwise, and refers to any condition characterized by the uncontrolled division of abnormal cells in the body. For example, mutations can occur in a cell that prevent it from being able to regulate cell division and result in the formation of one or more tumors. Cancers may be benign, pre-malignant or malignant. Cancer occurs in a variety of cells and tissues, including, but not limited to, the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, meningioma, etc.), breast, genital system, (e.g., uterus, ovary, prostate, testis, etc.), urinary system (e.g., bladder, kidney, ureter, etc.), eye, nervous system (e.g., brain, etc.), endocrine system (e.g., thyroid, etc.), soft tissues (e.g., muscle, fat, etc.), and hematopoietic system (e.g., lymphoma, myeloma, leukemia, acute lymphocytic leukemia, chronic lymphocytic leukemia, acute myeloid leukemia, chronic myeloid leukemia, etc.).

As used herein, the term “solid cancer” has its plain and ordinary meaning as understood in light of the specification, and, unless specified otherwise, and refers to a cancer that results in a malignant solid tumor. Solid tumors include sarcomas and carcinomas. Blood cancers do not typically form solid tumors.

As used herein, the term “unresectable” has its plain and ordinary meaning as understood in light of the specification, and, unless specified otherwise, and refers to a tumor that is unable to be treated by removal with surgery.

As used herein, the term “locally advanced” has its plain and ordinary meaning as understood in light of the specification, and, unless specified otherwise, and refers to a cancer that has high vascular involvement and/or has grown outside of the body part or organ it started in but has not yet metastasized.

As used herein, the term “refractory” has its plain and ordinary meaning as understood in light of the specification, and, unless specified otherwise, and refers to a cancer that does not respond to a treatment.

As used herein, the terms “treat,” “treating,” and “treatment” have their plain and ordinary meaning as understood in light of the specification, and, unless specified otherwise, and refer to therapeutic or preventative measures described herein. The methods of “treatment” employ administration of an antibody to a subject having a cancer in order to prevent, cure, delay, reduce the severity of, or ameliorate one or more symptoms of the cancer or recurring cancer, or in order to prolong the survival of a subject beyond that expected in the absence of such treatment. In some embodiments, “treatment” includes achieving complete response, partial response, or stable disease. In some embodiments, “treatment” includes achieving at least stable disease.

As used herein, the term “effective amount” has its plain and ordinary meaning as understood in light of the specification, and, unless specified otherwise, and in the context of the administration of a therapy to a subject refers to the amount of a therapy that achieves a desired prophylactic or therapeutic effect.

As used herein, the term “about” when referring to a measurable value, such as a dosage, encompasses variations of ±5% of a given value or range.

As used herein with respect to an antibody, the term “isolated” has its plain and ordinary meaning as understood in light of the specification, and, unless specified otherwise, and refers to an antibody that is separated from one or more contaminants (e.g., polypeptides, polynucleotides, lipids, host cells, or carbohydrates, etc.) which are present in a natural source of the antibody. All instances of “antibodies” described herein are additionally contemplated as antibodies that are, but need not be, isolated.

The determination of “percent identity” between two sequences (e.g., amino acid sequences or nucleic acid sequences) can be accomplished using a mathematical algorithm. A non-limiting example of a mathematical algorithm utilized for the comparison of two sequences is the algorithm of Karlin S & Altschul SF (1990) PNAS 87:2264-2268, modified as in Karlin S & Altschul SF (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted. In some embodiments, percent identity is calculated only using exact matches without introducing gaps.

As used herein, the term “amino acid change” has its plain and ordinary meaning as understood in light of the specification, and, unless specified otherwise, and refers to the substitution, insertion of an amino acid at a position in an amino acid sequence.

### Anti-DDR1 Antibodies

In an aspect, any antibody that specifically binds DDR1 (i.e., anti-DDR1 antibody) is useful in the methods and uses provided herein.

In an embodiment, the amino acid sequences of the CDR, VH/VL, and heavy chain and light chain sequences of exemplary antibodies that specifically bind to DDR1 are set forth in Tables 2, 3, and 4, respectively.

TABLE 2
CDR amino acid sequences of exemplary anti-DDR1 antibodies.

| | | SEQ | | SEQ | | SEQ |
|---|---|---|---|---|---|---|
| | | ID | | ID | | ID |
| Antibody | CDRL1 | NO: | CDRL2 | NO: | CDRL3 | NO: |
| humanized | QSIGSV | 4 | GVF | 5 | QYIPYGSSP | 6 |
| DDR1-9 | | | | | | |
| (9H-1) | | | | | | |
| | | SEQ | | SEQ | | SEQ |
| | | ID | | ID | | ID |
| | CDRH1 | NO: | CDRH2 | NO: | CDRH3 | NO: |
| humanized | GFSLNRYY | 1 | ISYGDTT | 2 | ARADTGDNGYLGLQ | 3 |
| DDR1-9 | | | | | L | |
| (9H-1) | | | | | | |

TABLE 3
VH/VL amino acid sequences of exemplary anti-DDR1 antibodies.

| | | |
|---|---|---|
| | SEQ | |
| | ID | |
| Antibody | NO: | Sequence |

Light chain variable region

| | | |
|---|---|---|
| humanized DDR1-9 | 8 | DIQMTQSPSSVSASVGDRVTITCQASQSIGSVLAWYQQKP |
| (9H-1)_1 | | GKAPKLLISGVFDLASGVPSRFSGSGSGTDFTLTISSLQPE |
| | | DFATYYCQYIPYGSSPFGGGTKVEIK |
| humanized DDR1-9 | 9 | DIQMTQSPSSVSASVGDRVTITCRASQSIGSVLAWYQQKP |
| (9H-1)_2 | | GKAPKLLIYGVFSLQSGVPSRFSGSGSGTDFTLTISSLQPE |
| | | DFATYYCQYIPYGSSPFGGGTKVEIK |

Heavy chain variable region

| | | |
|---|---|---|
| humanized DDR1-9 | 7 | QVQLVESGGRVVQPGRSLRLSCTASGFSLNRYYMLWVR |
| (9H-1) | | QAPGKGLEWIGTISYGDTTYYASWAKGRFTISRDNSKNT |
| | | LYLQMNSLRAEDTAVYYCARADTGDNGYLGLQLWGQG |
| | | TLVTVSS |

TABLE 4
LC/HC amino acid sequences of exemplary anti-DDR1 antibodies.

| | | |
|---|---|---|
| | SEQ ID | |
| Antibody | NO: | Sequence |

Light Chain

| | | |
|---|---|---|
| humanized | 12 | DIQMTQSPSSVSASVGDRVTITCQASQSIGSVLAWYQQKPGKAPKLLI |
| DDR1-9 | | SGVFDLASGVPSRFSGSGSGTDFTLTISSLQPEDFATYYCQYIPYGSSP |
| (9H-1)_1 | | FGGGTKVEIKRTVAAPSVFIFPPSDEQLKSGTASVVCLLNNFYPREAK |
| | | VQWKVDNALQSGNSQESVTEQDSKDSTYSLSSTLTLSKADYEKHKV |
| | | YACEVTHQGLSSPVTKSFNRGEC |

Heavy Chain

| | | |
|---|---|---|
| humanized | 10 | QVQLVESGGRVVQPGRSLRLSCTASGFSLNRYYMLWVRQAPGKGLE |
| DDR1-9 | | WIGTISYGDTTYYASWAKGRFTISRDNSKNTLYLQMNSLRAEDTAV |
| (9H-1)_1 | | YYCARADTGDNGYLGLQLWGQGTLVTVSSASTKGPSVFPLAPSSKS |
| inert IgG1 | | TSGGTAALGCLVKDYFPEPVTVSWNSGALTSGVHTFPAVLQSSGLYS |
| Fc | | LSSVVTVPSSSLGTQTYICNVNHKPSNTKVDKRVEPKSCDKTHTCPPC |
| | | PAPEAEGAPSVFLFPPKPKDTLMISRTPEVTCVVVDVSHEDPEVKFN |
| | | WYVDGVEVHNAKTKPREEQYNSTYRVVSVLTVLHQDWLNGKEYK |
| | | CKVSNKALPSSIEKTISKAKGQPREPQVYTLPPSREEMTKNQVSLTCL |
| | | VKGFYPSDIAVEWESNGQPENNYKTTPPVLDSDGSFFLYSKLTVDKS |
| | | RWQQGNVFSCSVMHEALHNHYTQKSLSLSPGK |
| humanized | 11 | QVQLVESGGRVVQPGRSLRLSCTASGFSLNRYYMLWVRQAPGKGLE |
| DDR1-9 | | WIGTISYGDTTYYASWAKGRFTISRDNSKNTLYLQMNSLRAEDTAV |
| (9H-1)_1 | | YYCARADTGDNGYLGLQLWGQGTLVTVSSASTKGPSVFPLAPSSKS |
| WT IgG1 | | TSGGTAALGCLVKDYFPEPVTVSWNSGALTSGVHTFPAVLQSSGLYS |
| Fc | | LSSVVTVPSSSLGTQTYICNVNHKPSNTKVDKRVEPKSCDKTHTCPPC |
| | | PAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVK |
| | | GFYPSDIAVEWESNGQPENNYKTTPPVLDSDGSFFLYSKLTVDKSRW |
| | | QQGNVFSCSVMHEALHNHYTQKSLSLSPGK |

In an embodiment, the antibody comprises: a heavy chain variable region (VH) comprising the CDRH1, CDRH2, and CDRH3 amino acid sequences of the VH amino acid sequence set forth in SEQ ID NO: 7, or a variant thereof comprising 1-5 amino acid changes in any one of the CDRH1, CDRH2, or CDRH3 amino acid sequences; and/or a light chain variable region (VL) comprising the CDRL1, CDRL2, and CDRL3 amino acid sequences of the VL amino acid sequence set forth in SEQ ID NO: 8 or 9, or a variant thereof comprising 1-5 amino acid changes in any one of the CDRL1, CDRL2, or CDRL3 amino acid sequences.

In an embodiment, (a) the VH comprises the CDRH1, CDRH2, and CDRH3 amino acid sequences, respectively, of: SEQ ID NO: 1, or a variant thereof comprising 1-5 amino acid changes, SEQ ID NO: 2, or a variant thereof comprising 1-3 amino acid changes, and SEQ ID NO: 3, or a variant thereof comprising 1-5 amino acid changes; and/or (b) the VL comprises the CDRL1, CDRL2, and CDRL3 amino acid sequences, respectively, of SEQ ID NO: 4, or a variant thereof comprising 1-5 amino acid changes, SEQ ID NO: 5, or a variant thereof comprising 1-5 amino acid changes, and SEQ ID NO: 6, or a variant thereof comprising 1-5 amino acid changes.

In an embodiment, (a) the VH comprises the CDRH1, CDRH2, and CDRH3 amino acid sequences, respectively, of: SEQ ID NO: 1, or a variant thereof comprising 1 or 2 amino acid changes, SEQ ID NO: 2, or a variant thereof comprising 1 or 2 amino acid changes, and SEQ ID NO: 3, or a variant thereof comprising 1 or 2 amino acid changes; and/or (b) the VL comprises the CDRL1, CDRL2, and CDRL3 amino acid sequences, respectively, of SEQ ID NO: 4, or a variant thereof comprising 1 or 2 amino acid changes, SEQ ID NO: 5, or a variant thereof comprising 1 or 2 amino acid changes, and SEQ ID NO: 6, or a variant thereof comprising 1 or 2 amino acid changes.

In an embodiment, (a) the VH comprises the CDRH1, CDRH2, and CDRH3 amino acid sequences, respectively, of: SEQ ID NO: 1, or a variant thereof comprising 1 amino acid change, SEQ ID NO: 2, or a variant thereof comprising 1 amino acid change, and SEQ ID NO: 3, or a variant thereof comprising 1 amino acid change; and/or (b) the VL comprises the CDRL1, CDRL2, and CDRL3 amino acid sequences, respectively, of SEQ ID NO: 4, or a variant thereof comprising 1 amino acid change, SEQ ID NO: 5, or a variant thereof comprising 1 amino acid change, and SEQ ID NO: 6, or a variant thereof comprising 1 amino acid change.

In an embodiment, the antibody comprises the CDRH1, CDRH2, CDRH3, CDRL1, CDRL2, and CDRL3 amino acid sequences set forth in SEQ ID NOs: 1, 2, 3, 4, 5, and 6, respectively.

In an embodiment, the antibody comprises: a VH comprising an amino acid sequence which is at least 75%, 80%, 85%, 90%, 95%, 96%, 97%, 98%, 99%, or 100% identical to the amino acid sequence set forth in SEQ ID NO: 7; and/or a VL comprising an amino acid sequence which is at least 75%, 80%, 85%, 90%, 95%, 96%, 97%, 98%, 99%, or 100% identical to the amino acid sequence set forth in SEQ ID NO: 8 or 9.

In an embodiment, the antibody comprises a VH comprising the amino acid sequence set forth in SEQ ID NO: 7 and a VL comprising the amino acid sequence set forth in SEQ ID NO: 8. In an embodiment, the amino acid sequence of the VH consists of the amino acid sequence set forth in SEQ ID NO: 7 and the amino acid sequence of VL consists of the amino acid sequence set forth in SEQ ID NO: 8.

In an embodiment, the antibody comprises a VH comprising the amino acid sequence set forth in SEQ ID NO: 7 and a VL comprising the amino acid sequence set forth in SEQ ID NO: 9. In an embodiment, the amino acid sequence of the VH consists of the amino acid sequence set forth in SEQ ID NO: 7 and the amino acid sequence of VL consists of the amino acid sequence set forth in SEQ ID NO: 9.

In an embodiment, the antibody comprises a heavy chain comprising an amino acid sequence which is at least 75%, 80%, 85%, 90%, 95%, 96%, 97%, 98%, 99%, or 100% identical to the amino acid sequence set forth in SEQ ID NO: 10 or 11 and/or a light chain comprising an amino acid sequence which is at least 75%, 80%, 85%, 90%, 95%, 96%, 97%, 98%, 99%, or 100% identical to the amino acid sequence set forth in SEQ ID NO: 12.

In an embodiment, the antibody comprises a heavy chain comprising the amino acid sequence set forth in SEQ ID NO: 10 and a light chain comprising the amino acid sequence set forth in SEQ ID NO: 12. In some embodiments, the antibody comprises a heavy chain comprising the amino acid sequence set forth in SEQ ID NO: 10, without the terminal lysine, and a light chain comprising the amino acid sequence set forth in SEQ ID NO: 12. In an embodiment, the amino acid sequence of the heavy chain consists of the amino acid sequence set forth in SEQ ID NO: 10 and the amino acid sequence of the light chain consists of the amino acid sequence set forth in SEQ ID NO: 12. In some embodiments, the amino acid sequence of the heavy chain consists of the amino acid sequence set forth in SEQ ID NO: 10, without the terminal lysine, and the amino acid sequence of the light chain consists of the amino acid sequence set forth in SEQ ID NO: 12.

In an embodiment, the antibody comprises a heavy chain comprising the amino acid sequence set forth in SEQ ID NO: 11 and a light chain comprising the amino acid sequence set forth in SEQ ID NO: 12. In some embodiments, the amino acid sequence of the heavy chain comprises the amino acid sequence set forth in SEQ ID NO: 11, without the terminal lysine, and the amino acid sequence of the light chain consists of the amino acid sequence set forth in SEQ ID NO: 12. In an embodiment, the amino acid sequence of the heavy chain consists of the amino acid sequence set forth in SEQ ID NO: 11 and the amino acid sequence of the light chain consists of the amino acid sequence set forth in SEQ ID NO: 12, referred to herein as the antibody “9H-1.” In some embodiments, the heavy chain of 9H-1 does not include the terminal lysine of SEQ ID NO: 11.

In some embodiments, the antibody is 9H-1 (PRTH-101), provided by Incendia Therapeutics, Inc. (Boston, MA).

Also provided herein are compositions (e.g., pharmaceutical compositions) that include at least one anti-DDR1 antibody of the present disclosure. A composition can include an antibody that specifically binds to human discoidin domain receptor tyrosine kinase 1 (DDR1); and a pharmaceutically acceptable carrier. Any suitable pharmaceutically acceptable carrier useful can be used. Remington's Pharmaceutical Sciences, by E. W. Martin, Mack Publishing Co., Easton, PA, 15th Edition (1975), describes compositions and formulations suitable for pharmaceutical delivery of the antibodies disclosed herein. In general, the nature of the carrier will depend on the particular mode of administration being employed. For instance, parenteral formulations can comprise injectable fluids that include pharmaceutically and physiologically acceptable fluids such as water, physiological saline, balanced salt solutions, aqueous dextrose, glycerol or the like as a vehicle. In addition to biologically neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate.

A composition of the present disclosure can take the form of solutions, suspensions, emulsion, and the like. Examples of suitable pharmaceutical agents are described in “Remington's Pharmaceutical Sciences.” Such compositions can contain a prophylactically or therapeutically effective amount of the antibody or fragment thereof, preferably in purified form, together with a suitable amount of carrier so as to provide the form for proper administration to the patient. The formulation may suit the mode of administration, which can be oral, intravenous, intraarterial, intrabuccal, intranasal, nebulized, bronchial inhalation, or delivered by mechanical ventilation.

Antibodies of the present disclosure, as described herein, can be formulated for parenteral administration, e.g., formulated for injection via the intradermal, intravenous, intramuscular, subcutaneous, intra-tumoral or even intraperitoneal routes. The antibodies could alternatively be administered by a topical route directly to the mucosa, for example by nasal drops, inhalation, or by nebulizer.

Generally, the ingredients of compositions of the disclosure can be supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### Methods and Dosage Regimens

The instant disclosure is directed to methods for reducing immune exclusion of a tumor and methods for reducing tumor burden in a subject in need thereof with an antibody that specifically binds to human DDR1 (i.e., an anti-DDR1 antibody). Also provided herein are particular dosage regimens for administering an anti-DDR1 antibody that results in a reduced tumor size and increased response to immunotherapy in subjects with solid cancers, e.g., colorectal cancer, ovarian cancer, or non-small cell lung cancer.

In an aspect, provided herein is a method of reducing immune exclusion of a tumor in a subject in need thereof, the method comprising administering to the subject 5 mg to 2000 mg of an antibody that specifically binds to human discoidin domain receptor tyrosine kinase 1 (DDR1).

In an aspect, provided herein is a method of reducing tumor burden in a subject in need thereof, the method comprising administering to the subject 5 mg to 2000 mg of an antibody that specifically binds to human discoidin domain receptor tyrosine kinase 1 (DDR1).

In an embodiment, the subject has cancer. In an embodiment, the antibody treats the cancer in the subject.

In an embodiment, the antibody is administered at a dose of about 5 mg to about 2000 mg. In an embodiment, the antibody is administered at a dose of about 8 mg to about 1600 mg. In an embodiment, the antibody is administered at a dose of about 8 mg to about 800 mg. In an embodiment, the antibody is administered at a dose of about 8 mg, about 9 mg, about 10 mg, about 15 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, or about 1600 mg. In an embodiment, the antibody is administered at a dose of about 8 mg, about 24 mg, about 25 mg, about 75 mg, about 80 mg, about 240 mg, about 250 mg, about 400 mg, about 800 mg, or about 1600 mg.

In an embodiment, the antibody is administered at a dose of 5 mg to 2000 mg. In an embodiment, the antibody is administered at a dose of 8 mg to 1600 mg. In an embodiment, the antibody is administered at a dose of 8 mg to 800 mg. In an embodiment, the antibody is administered at a dose of 8 mg, 9 mg, 10 mg, 15 mg, 20 mg, 21 mg, 22 mg, 23 mg, 24 mg, 25 mg, 30 mg, 35 mg, 40 mg, 45 mg, 50 mg, 55 mg, 60 mg, 65 mg, 70 mg, 75 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, or 1600 mg. In an embodiment, the antibody is administered at a dose of 8 mg, 24 mg, 25 mg, 75 mg, 80 mg, 240 mg, 250 mg, 400 mg, 800 mg, or 1600 mg.

In an embodiment, the antibody is administered intravenously or subcutaneously.

In an embodiment, the antibody is administered once weekly. In some embodiments, the antibody is administered no more frequently than once weekly. In an embodiment, the antibody is administered once every 2 weeks. In some embodiments, the antibody is administered no more frequently than once every 2 weeks. In an embodiment, the antibody is administered once every 3 weeks. In some embodiments, the antibody is administered no more frequently than once every 3 weeks. In an embodiment, the antibody is administered once every 4 weeks. In some embodiments, the antibody is administered no more frequently than once every 4 weeks. In an embodiment, the antibody is administered once every 5 weeks. In some embodiments, the antibody is administered no more frequently than once every 5 weeks. In an embodiment, the antibody is administered once every 6 weeks. In some embodiments, the antibody is administered no more frequently than once every 6 weeks. In an embodiment, the antibody is administered once every 7 weeks. In an embodiment, the antibody is administered once every 8 weeks. In some embodiments, the antibody is administered no more frequently than once every 8 weeks.

In an embodiment, the antibody is administered at a dose of about 5 mg to about 2000 mg once weekly. In an embodiment, the antibody is administered at a dose of about 8 mg to about 1600 mg once weekly. In an embodiment, the antibody is administered at a dose of about 8 mg to about 800 mg once weekly. In an embodiment, the antibody is administered at a dose of about 8 mg, about 9 mg, about 10 mg, about 15 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, or about 1600 mg once weekly. In an embodiment, the antibody is administered at a dose of about 8 mg, about 24 mg, about 25 mg, about 75 mg, about 80 mg, about 240 mg, about 250 mg, about 400 mg, about 800 mg, or about 1600 mg once weekly.

In an embodiment, the antibody is administered at a dose of 5 mg to 2000 mg once weekly. In an embodiment, the antibody is administered at a dose of 8 mg to 1600 mg once weekly. In an embodiment, the antibody is administered at a dose of 8 mg to 800 mg once weekly. In an embodiment, the antibody is administered at a dose of 8 mg, 9 mg, 10 mg, 15 mg, 20 mg, 21 mg, 22 mg, 23 mg, 24 mg, 25 mg, 30 mg, 35 mg, 40 mg, 45 mg, 50 mg, 55 mg, 60 mg, 65 mg, 70 mg, 75 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, or 1600 mg once weekly. In an embodiment, the antibody is administered at a dose of 8 mg, 24 mg, 25 mg, 75 mg, 80 mg, 240 mg, 250 mg, 400 mg, 800 mg, or 1600 mg once weekly.

In an embodiment, the antibody is administered at a dose of about 5 mg to about 2000 mg once every 2 weeks. In an embodiment, the antibody is administered at a dose of about 8 mg to about 1600 mg once every 2 weeks. In an embodiment, the antibody is administered at a dose of about 8 mg to about 800 mg once every 2 weeks. In an embodiment, the antibody is administered at a dose of about 8 mg, about 9 mg, about 10 mg, about 15 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, or about 1600 mg once every 2 weeks. In an embodiment, the antibody is administered at a dose of 8 mg, about 24 mg, about 25 mg, about 75 mg, about 80 mg, about 240 mg, about 250 mg, about 400 mg, about 800 mg, or about 1600 mg once every 2 weeks.

In an embodiment, the antibody is administered at a dose of 5 mg to 2000 mg once every 2 weeks. In an embodiment, the antibody is administered at a dose of 8 mg to 1600 mg once every 2 weeks. In an embodiment, the antibody is administered at a dose of 8 mg to 800 mg once every 2 weeks. In an embodiment, the antibody is administered at a dose of 8 mg, 9 mg, 10 mg, 15 mg, 20 mg, 21 mg, 22 mg, 23 mg, 24 mg, 25 mg, 30 mg, 35 mg, 40 mg, 45 mg, 50 mg, 55 mg, 60 mg, 65 mg, 70 mg, 75 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, or 1600 mg once every 2 weeks. In an embodiment, the antibody is administered at a dose of 8 mg, 24 mg, 25 mg, 75 mg, 80 mg, 240 mg, 250 mg, 400 mg, 800 mg, or 1600 mg once every 2 weeks.

In an embodiment, the antibody is administered at a dose of about 5 mg to about 2000 mg once every 3 weeks. In an embodiment, the antibody is administered at a dose of about 8 mg to about 1600 mg once every 3 weeks. In an embodiment, the antibody is administered at a dose of about 8 mg to about 800 mg once every 3 weeks. In an embodiment, the antibody is administered at a dose of about 8 mg, about 9 mg, about 10 mg, about 15 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, or about 1600 mg once every 3 weeks. In an embodiment, the antibody is administered at a dose of about 8 mg, about 24 mg, about 25 mg, about 75 mg, about 80 mg, about 240 mg, about 250 mg, about 400 mg, about 800 mg, or about 1600 mg once every 3 weeks.

In an embodiment, the antibody is administered at a dose of 5 mg to 2000 mg once every 3 weeks. In an embodiment, the antibody is administered at a dose of 8 mg to 1600 mg once every 3 weeks. In an embodiment, the antibody is administered at a dose of 8 mg to 800 mg once every 3 weeks. In an embodiment, the antibody is administered at a dose of 8 mg, 9 mg, 10 mg, 15 mg, 20 mg, 21 mg, 22 mg, 23 mg, 24 mg, 25 mg, 30 mg, 35 mg, 40 mg, 45 mg, 50 mg, 55 mg, 60 mg, 65 mg, 70 mg, 75 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, or 1600 mg once every 3 weeks. In an embodiment, the antibody is administered at a dose of 8 mg, 24 mg, 25 mg, 75 mg, 80 mg, 240 mg, 250 mg, 400 mg, 800 mg, or 1600 mg once every 3 weeks.

In an embodiment, the antibody is administered at a dose of about 5 mg to about 2000 mg once every 4 weeks. In an embodiment, the antibody is administered at a dose of about 8 mg to about 1600 mg once every 4 weeks. In an embodiment, the antibody is administered at a dose of about 8 mg to about 800 mg once every 4 weeks. In an embodiment, the antibody is administered at a dose of about 8 mg, about 9 mg, about 10 mg, about 15 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, or about 1600 mg once every 4 weeks. In an embodiment, the antibody is administered at a dose of about 8 mg, about 24 mg, about 25 mg, about 75 mg, about 80 mg, about 240 mg, about 250 mg, about 400 mg, about 800 mg, or about 1600 mg once every 4 weeks.

In an embodiment, the antibody is administered at a dose of 5 mg to 2000 mg once every 4 weeks. In an embodiment, the antibody is administered at a dose of 8 mg to 1600 mg once every 4 weeks. In an embodiment, the antibody is administered at a dose of 8 mg to 800 mg once every 4 weeks. In an embodiment, the antibody is administered at a dose of 8 mg, 9 mg, 10 mg, 15 mg, 20 mg, 21 mg, 22 mg, 23 mg, 24 mg, 25 mg, 30 mg, 35 mg, 40 mg, 45 mg, 50 mg, 55 mg, 60 mg, 65 mg, 70 mg, 75 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, or 1600 mg once every 4 weeks. In an embodiment, the antibody is administered at a dose of 8 mg, 24 mg, 25 mg, 75 mg, 80 mg, 240 mg, 250 mg, 400 mg, 800 mg, or 1600 mg once every 4 weeks.

In an embodiment, the antibody is administered at a dose of about 5 mg to about 2000 mg once every 5 weeks. In an embodiment, the antibody is administered at a dose of about 8 mg to about 1600 mg once every 5 weeks. In an embodiment, the antibody is administered at a dose of about 8 mg to about 800 mg once every 5 weeks. In an embodiment, the antibody is administered at a dose of about 8 mg, about 9 mg, about 10 mg, about 15 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, or about 1600 mg once every 5 weeks. In an embodiment, the antibody is administered at a dose of about 8 mg, about 24 mg, about 25 mg, about 75 mg, about 80 mg, about 240 mg, about 250 mg, about 400 mg, about 800 mg, or about 1600 mg once every 5 weeks.

In an embodiment, the antibody is administered at a dose of 5 mg to 2000 mg once every 5 weeks. In an embodiment, the antibody is administered at a dose of 8 mg to 1600 mg once every 5 weeks. In an embodiment, the antibody is administered at a dose of 8 mg to 800 mg once every 5 weeks. In an embodiment, the antibody is administered at a dose of 8 mg, 9 mg, 10 mg, 15 mg, 20 mg, 21 mg, 22 mg, 23 mg, 24 mg, 25 mg, 30 mg, 35 mg, 40 mg, 45 mg, 50 mg, 55 mg, 60 mg, 65 mg, 70 mg, 75 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, or 1600 mg once every 5 weeks. In an embodiment, the antibody is administered at a dose of 8 mg, 24 mg, 25 mg, 75 mg, 80 mg, 240 mg, 250 mg, 400 mg, 800 mg, or 1600 mg once every 5 weeks.

In an embodiment, the antibody is administered at a dose of about 5 mg to about 2000 mg once every 6 weeks. In an embodiment, the antibody is administered at a dose of about 8 mg to about 1600 mg once every 6 weeks. In an embodiment, the antibody is administered at a dose of about 8 mg to about 800 mg once every 6 weeks. In an embodiment, the antibody is administered at a dose of about 8 mg, about 9 mg, about 10 mg, about 15 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, or about 1600 mg once every 6 weeks. In an embodiment, the antibody is administered at a dose of about 8 mg, about 24 mg, about 25 mg, about 75 mg, about 80 mg, about 240 mg, about 250 mg, about 400 mg, about 800 mg, or about 1600 mg once every 6 weeks.

In an embodiment, the antibody is administered at a dose of 5 mg to 2000 mg once every 6 weeks. In an embodiment, the antibody is administered at a dose of 8 mg to 1600 mg once every 6 weeks. In an embodiment, the antibody is administered at a dose of 8 mg to 800 mg once every 6 weeks. In an embodiment, the antibody is administered at a dose of 8 mg, 9 mg, 10 mg, 15 mg, 20 mg, 21 mg, 22 mg, 23 mg, 24 mg, 25 mg, 30 mg, 35 mg, 40 mg, 45 mg, 50 mg, 55 mg, 60 mg, 65 mg, 70 mg, 75 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, or 1600 mg once every 6 weeks. In an embodiment, the antibody is administered at a dose of 8 mg, 24 mg, 25 mg, 75 mg, 80 mg, 240 mg, 250 mg, 400 mg, 800 mg, or 1600 mg once every 6 weeks.

In an embodiment, the antibody is administered at a dose of about 5 mg to about 2000 mg once every 7 weeks. In an embodiment, the antibody is administered at a dose of about 8 mg to about 1600 mg once every 7 weeks. In an embodiment, the antibody is administered at a dose of about 8 mg to about 800 mg once every 7 weeks. In an embodiment, the antibody is administered at a dose of about 8 mg, about 9 mg, about 10 mg, about 15 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, or about 1600 mg once every 7 weeks. In an embodiment, the antibody is administered at a dose of about 8 mg, about 24 mg, about 25 mg, about 75 mg, about 80 mg, about 240 mg, about 250 mg, about 400 mg, about 800 mg, or about 1600 mg once every 7 weeks.

In an embodiment, the antibody is administered at a dose of 5 mg to 2000 mg once every 7 weeks. In an embodiment, the antibody is administered at a dose of 8 mg to 1600 mg once every 7 weeks. In an embodiment, the antibody is administered at a dose of 8 mg to 800 mg once every 7 weeks. In an embodiment, the antibody is administered at a dose of 8 mg, 9 mg, 10 mg, 15 mg, 20 mg, 21 mg, 22 mg, 23 mg, 24 mg, 25 mg, 30 mg, 35 mg, 40 mg, 45 mg, 50 mg, 55 mg, 60 mg, 65 mg, 70 mg, 75 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, or 1600 mg once every 7 weeks. In an embodiment, the antibody is administered at a dose of 8 mg, 24 mg, 25 mg, 75 mg, 80 mg, 240 mg, 250 mg, 400 mg, 800 mg, or 1600 mg once every 7 weeks.

In an embodiment, the antibody is administered at a dose of about 5 mg to about 2000 mg once every 8 weeks. In an embodiment, the antibody is administered at a dose of about 8 mg to about 1600 mg once every 8 weeks. In an embodiment, the antibody is administered at a dose of about 8 mg to about 800 mg once every 8 weeks. In an embodiment, the antibody is administered at a dose of about 8 mg, about 9 mg, about 10 mg, about 15 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, or about 1600 mg once every 8 weeks. In an embodiment, the antibody is administered at a dose of about 8 mg, about 24 mg, about 25 mg, about 75 mg, about 80 mg, about 240 mg, about 250 mg, about 400 mg, about 800 mg, or about 1600 mg once every 8 weeks.

In an embodiment, the antibody is administered at a dose of 5 mg to 2000 mg once every 8 weeks. In an embodiment, the antibody is administered at a dose of 8 mg to 1600 mg once every 8 weeks. In an embodiment, the antibody is administered at a dose of 8 mg to 800 mg once every 8 weeks. In an embodiment, the antibody is administered at a dose of 8 mg, 9 mg, 10 mg, 15 mg, 20 mg, 21 mg, 22 mg, 23 mg, 24 mg, 25 mg, 30 mg, 35 mg, 40 mg, 45 mg, 50 mg, 55 mg, 60 mg, 65 mg, 70 mg, 75 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, or 1600 mg once every 8 weeks. In an embodiment, the antibody is administered at a dose of 8 mg, 24 mg, 25 mg, 75 mg, 80 mg, 240 mg, 250 mg, 400 mg, 800 mg, or 1600 mg once every 8 weeks.

In an embodiment, the antibody is administered intravenously at a dose of 8 mg once every 3 weeks. In an embodiment, the antibody is administered intravenously at a dose of 24 mg once every 3 weeks. In an embodiment, the antibody is administered intravenously at a dose of 25 mg once every 3 weeks. In an embodiment, the antibody is administered intravenously at a dose of 75 mg once every 3 weeks. In an embodiment, the antibody is administered intravenously at a dose of 80 mg once every 3 weeks. In an embodiment, the antibody is administered intravenously at a dose of 240 mg once every 3 weeks. In an embodiment, the antibody is administered intravenously at a dose of 250 mg once every 3 weeks. In an embodiment, the antibody is administered intravenously at a dose of 400 mg once every 3 weeks. In an embodiment, the antibody is administered intravenously at a dose of 800 mg once every 3 weeks. In an embodiment, the antibody is administered intravenously at a dose of 1600 mg once every 3 weeks.

In an embodiment, the antibody is administered intravenously. In an embodiment, the antibody is administered via intravenous infusion over 60 minutes. In an embodiment, the antibody is administered via intravenous infusion over 30 minutes. In some embodiments, the antibody is administered intra-tumoraly.

In an embodiment, the dose is a therapeutically effective amount.

In some embodiments, the antibody is administered using a flat dose (e.g., the administered amount of the antibody is not based on the body weight of the subject).

In an aspect, provided herein is a method of reducing immune exclusion of a tumor in a subject in need thereof, the method comprising administering to the subject 0.1 mg/kg to 100 mg/kg of an antibody that specifically binds to human discoidin domain receptor tyrosine kinase 1 (DDR1).

In an aspect, provided herein is a method of reducing tumor burden in a subject in need thereof, the method comprising administering to the subject 0.1 mg/kg to 100 mg/kg of an antibody that specifically binds to human discoidin domain receptor tyrosine kinase 1 (DDR1).

In an embodiment, the subject has cancer. In an embodiment, the antibody treats the cancer in the subject.

In an embodiment, the antibody is administered at a dose of about 0.1 mg/kg to about 100 mg/kg. In an embodiment, the antibody is administered at a dose of about 0.1 mg/kg to about 50 mg/kg. In an embodiment, the antibody is administered at a dose of about 0.1 mg/kg to about 25 mg/kg. In an embodiment, the antibody is administered at a dose of about 0.1 mg/kg to about 20 mg/kg. In an embodiment, the antibody is administered at a dose of about 0.1 mg/kg, about 0.2 mg/kg, about 0.3 mg/kg, about 0.4 mg/kg, about 0.5 mg/kg, about 0.75 mg/kg, about 1 mg/kg, about 2 mg/kg, about 3 mg/kg, about 4 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg. In an embodiment, the antibody is administered at a dose of about 0.1 mg/kg, 0.3 mg/kg, 1 mg/kg, 3 mg/kg, 10 mg/kg, or 20 mg/kg.

In an embodiment, the antibody is administered at a dose of 0.1 mg/kg to 100 mg/kg. In an embodiment, the antibody is administered at a dose of 0.1 mg/kg to 50 mg/kg. In an embodiment, the antibody is administered at a dose of 0.1 mg/kg to 25 mg/kg. In an embodiment, the antibody is administered at a dose of 0.1 mg/kg to 20 mg/kg. In an embodiment, the antibody is administered at a dose of 0.1 mg/kg, 0.2 mg/kg, 0.3 mg/kg, 0.4 mg/kg, 0.5 mg/kg, 0.75 mg/kg, 1 mg/kg, 2 mg/kg, 3 mg/kg, 4 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. In an embodiment, the antibody is administered at a dose of 0.1 mg/kg, 0.3 mg/kg, 1 mg/kg, 3 mg/kg, 10 mg/kg, or 20 mg/kg.

In an embodiment, the antibody is administered intravenously or subcutaneously. In some embodiments, the antibody is administered intra-tumoraly.

In an embodiment, the antibody is administered once weekly. In some embodiments, the antibody is administered no more frequently than once weekly. In an embodiment, the antibody is administered once every 2 weeks. In some embodiments, the antibody is administered no more frequently than once every 2 weeks. In an embodiment, the antibody is administered once every 3 weeks. In some embodiments, the antibody is administered no more frequently than once every 3 weeks. In an embodiment, the antibody is administered once every 4 weeks. In some embodiments, the antibody is administered no more frequently than once every 4 weeks. In an embodiment, the antibody is administered once every 5 weeks. In some embodiments, the antibody is administered no more frequently than once every 5 weeks. In an embodiment, the antibody is administered once every 6 weeks. In some embodiments, the antibody is administered no more frequently than once every 6 weeks. In an embodiment, the antibody is administered once every 7 weeks. In some embodiments, the antibody is administered no more frequently than once every 7 weeks. In an embodiment, the antibody is administered once every 8 weeks. In some embodiments, the antibody is administered no more frequently than once every 8 weeks.

In an embodiment, the antibody is administered at a dose of about 0.1 mg/kg to about 100 mg/kg once weekly. In an embodiment, the antibody is administered at a dose of about 0.1 mg/kg to about 50 mg/kg once weekly. In an embodiment, the antibody is administered at a dose of about 0.1 mg/kg to about 25 mg/kg once weekly. In an embodiment, the antibody is administered at a dose of about 0.1 mg/kg to about 20 mg/kg once weekly. In an embodiment, the antibody is administered at a dose of about 0.1 mg/kg, about 0.2 mg/kg, about 0.3 mg/kg, about 0.4 mg/kg, about 0.5 mg/kg, about 0.75 mg/kg, about 1 mg/kg, about 2 mg/kg, about 3 mg/kg, about 4 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg once weekly. In an embodiment, the antibody is administered at a dose of about 0.1 mg/kg, about 0.3 mg/kg, about 1 mg/kg, about 3 mg/kg, about 10 mg/kg, or about 20 mg/kg once weekly.

In an embodiment, the antibody is administered at a dose of 0.1 mg/kg to 100 mg/kg once weekly. In an embodiment, the antibody is administered at a dose of 0.1 mg/kg to 50 mg/kg once weekly. In an embodiment, the antibody is administered at a dose of 0.1 mg/kg to 25 mg/kg once weekly. In an embodiment, the antibody is administered at a dose of 0.1 mg/kg to 20 mg/kg once weekly. In an embodiment, the antibody is administered at a dose of 0.1 mg/kg, 0.2 mg/kg, 0.3 mg/kg, 0.4 mg/kg, 0.5 mg/kg, 0.75 mg/kg, 1 mg/kg, 2 mg/kg, 3 mg/kg, 4 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once weekly. In an embodiment, the antibody is administered at a dose of 0.1 mg/kg, 0.3 mg/kg, 1 mg/kg, 3 mg/kg, 10 mg/kg, or 20 mg/kg once weekly.

In an embodiment, the antibody is administered at a dose of about 0.1 mg/kg to about 100 mg/kg once every 2 weeks. In an embodiment, the antibody is administered at a dose of about 0.1 mg/kg to about 50 mg/kg once every 2 weeks. In an embodiment, the antibody is administered at a dose of about 0.1 mg/kg to about 25 mg/kg once every 2 weeks. In an embodiment, the antibody is administered at a dose of about 0.1 mg/kg to about 20 mg/kg once every 2 weeks. In an embodiment, the antibody is administered at a dose of about 0.1 mg/kg, about 0.2 mg/kg, about 0.3 mg/kg, about 0.4 mg/kg, about 0.5 mg/kg, about 0.75 mg/kg, about 1 mg/kg, about 2 mg/kg, about 3 mg/kg, about 4 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg once every 2 weeks. In an embodiment, the antibody is administered at a dose of about 0.1 mg/kg, about 0.3 mg/kg, about 1 mg/kg, about 3 mg/kg, about 10 mg/kg, or about 20 mg/kg once every 2 weeks.

In an embodiment, the antibody is administered at a dose of 0.1 mg/kg to 100 mg/kg once every 2 weeks. In an embodiment, the antibody is administered at a dose of 0.1 mg/kg to 50 mg/kg once every 2 weeks. In an embodiment, the antibody is administered at a dose of 0.1 mg/kg to 25 mg/kg once every 2 weeks. In an embodiment, the antibody is administered at a dose of 0.1 mg/kg to 20 mg/kg once every 2 weeks. In an embodiment, the antibody is administered at a dose of 0.1 mg/kg, 0.2 mg/kg, 0.3 mg/kg, 0.4 mg/kg, 0.5 mg/kg, 0.75 mg/kg, 1 mg/kg, 2 mg/kg, 3 mg/kg, 4 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 2 weeks. In an embodiment, the antibody is administered at a dose of 0.1 mg/kg, 0.3 mg/kg, 1 mg/kg, 3 mg/kg, 10 mg/kg, or 20 mg/kg once every 2 weeks.

In an embodiment, the antibody is administered at a dose of about 0.1 mg/kg to about 100 mg/kg once every 3 weeks. In an embodiment, the antibody is administered at a dose of about 0.1 mg/kg to about 50 mg/kg once every 3 weeks. In an embodiment, the antibody is administered at a dose of about 0.1 mg/kg to about 25 mg/kg once every 3 weeks. In an embodiment, the antibody is administered at a dose of about 0.1 mg/kg to about 20 mg/kg once every 3 weeks. In an embodiment, the antibody is administered at a dose of about 0.1 mg/kg, about 0.2 mg/kg, about 0.3 mg/kg, about 0.4 mg/kg, about 0.5 mg/kg, about 0.75 mg/kg, about 1 mg/kg, about 2 mg/kg, about 3 mg/kg, about 4 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg once every 3 weeks. In an embodiment, the antibody is administered at a dose of about 0.1 mg/kg, about 0.3 mg/kg, about 1 mg/kg, about 3 mg/kg, about 10 mg/kg, or about 20 mg/kg once every 3 weeks.

In an embodiment, the antibody is administered at a dose of 0.1 mg/kg to 100 mg/kg once every 3 weeks. In an embodiment, the antibody is administered at a dose of 0.1 mg/kg to 50 mg/kg once every 3 weeks. In an embodiment, the antibody is administered at a dose of 0.1 mg/kg to 25 mg/kg once every 3 weeks. In an embodiment, the antibody is administered at a dose of 0.1 mg/kg to 20 mg/kg once every 3 weeks. In an embodiment, the antibody is administered at a dose of 0.1 mg/kg, 0.2 mg/kg, 0.3 mg/kg, 0.4 mg/kg, 0.5 mg/kg, 0.75 mg/kg, 1 mg/kg, 2 mg/kg, 3 mg/kg, 4 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 3 weeks. In an embodiment, the antibody is administered at a dose of 0.1 mg/kg, 0.3 mg/kg, 1 mg/kg, 3 mg/kg, 10 mg/kg, or 20 mg/kg once every 3 weeks.

In an embodiment, the antibody is administered at a dose of about 0.1 mg/kg to about 100 mg/kg once every 4 weeks. In an embodiment, the antibody is administered at a dose of about 0.1 mg/kg to about 50 mg/kg once every 4 weeks. In an embodiment, the antibody is administered at a dose of about 0.1 mg/kg to about 25 mg/kg once every 4 weeks. In an embodiment, the antibody is administered at a dose of about 0.1 mg/kg to about 20 mg/kg once every 4 weeks. In an embodiment, the antibody is administered at a dose of about 0.1 mg/kg, about 0.2 mg/kg, about 0.3 mg/kg, about 0.4 mg/kg, about 0.5 mg/kg, about 0.75 mg/kg, about 1 mg/kg, about 2 mg/kg, about 3 mg/kg, about 4 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg once every 4 weeks. In an embodiment, the antibody is administered at a dose of about 0.1 mg/kg, about 0.3 mg/kg, about 1 mg/kg, about 3 mg/kg, about 10 mg/kg, or about 20 mg/kg once every 4 weeks.

In an embodiment, the antibody is administered at a dose of 0.1 mg/kg to 100 mg/kg once every 4 weeks. In an embodiment, the antibody is administered at a dose of 0.1 mg/kg to 50 mg/kg once every 4 weeks. In an embodiment, the antibody is administered at a dose of 0.1 mg/kg to 25 mg/kg once every 4 weeks. In an embodiment, the antibody is administered at a dose of 0.1 mg/kg to 20 mg/kg once every 4 weeks. In an embodiment, the antibody is administered at a dose of 0.1 mg/kg, 0.2 mg/kg, 0.3 mg/kg, 0.4 mg/kg, 0.5 mg/kg, 0.75 mg/kg, 1 mg/kg, 2 mg/kg, 3 mg/kg, 4 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 4 weeks. In an embodiment, the antibody is administered at a dose of 0.1 mg/kg, 0.3 mg/kg, 1 mg/kg, 3 mg/kg, 10 mg/kg, or 20 mg/kg once every 4 weeks.

In an embodiment, the antibody is administered at a dose of about 0.1 mg/kg to about 100 mg/kg once every 5 weeks. In an embodiment, the antibody is administered at a dose of about 0.1 mg/kg to about 50 mg/kg once every 5 weeks. In an embodiment, the antibody is administered at a dose of about 0.1 mg/kg to about 25 mg/kg once every 5 weeks. In an embodiment, the antibody is administered at a dose of about 0.1 mg/kg to about 20 mg/kg once every 5 weeks. In an embodiment, the antibody is administered at a dose of about 0.1 mg/kg, about 0.2 mg/kg, about 0.3 mg/kg, about 0.4 mg/kg, about 0.5 mg/kg, about 0.75 mg/kg, about 1 mg/kg, about 2 mg/kg, about 3 mg/kg, about 4 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg once every 5 weeks. In an embodiment, the antibody is administered at a dose of about 0.1 mg/kg, about 0.3 mg/kg, about 1 mg/kg, about 3 mg/kg, about 10 mg/kg, or about 20 mg/kg once every 5 weeks.

In an embodiment, the antibody is administered at a dose of 0.1 mg/kg to 100 mg/kg once every 5 weeks. In an embodiment, the antibody is administered at a dose of 0.1 mg/kg to 50 mg/kg once every 5 weeks. In an embodiment, the antibody is administered at a dose of 0.1 mg/kg to 25 mg/kg once every 5 weeks. In an embodiment, the antibody is administered at a dose of 0.1 mg/kg to 20 mg/kg once every 5 weeks. In an embodiment, the antibody is administered at a dose of 0.1 mg/kg, 0.2 mg/kg, 0.3 mg/kg, 0.4 mg/kg, 0.5 mg/kg, 0.75 mg/kg, 1 mg/kg, 2 mg/kg, 3 mg/kg, 4 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 5 weeks. In an embodiment, the antibody is administered at a dose of 0.1 mg/kg, 0.3 mg/kg, 1 mg/kg, 3 mg/kg, 10 mg/kg, or 20 mg/kg once every 5 weeks.

In an embodiment, the antibody is administered at a dose of about 0.1 mg/kg to about 100 mg/kg once every 6 weeks. In an embodiment, the antibody is administered at a dose of about 0.1 mg/kg to about 50 mg/kg once every 6 weeks. In an embodiment, the antibody is administered at a dose of about 0.1 mg/kg to about 25 mg/kg once every 6 weeks. In an embodiment, the antibody is administered at a dose of about 0.1 mg/kg to about 20 mg/kg once every 6 weeks. In an embodiment, the antibody is administered at a dose of about 0.1 mg/kg, about 0.2 mg/kg, about 0.3 mg/kg, about 0.4 mg/kg, about 0.5 mg/kg, about 0.75 mg/kg, about 1 mg/kg, about 2 mg/kg, about 3 mg/kg, about 4 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg once every 6 weeks. In an embodiment, the antibody is administered at a dose of about 0.1 mg/kg, about 0.3 mg/kg, about 1 mg/kg, about 3 mg/kg, about 10 mg/kg, or about 20 mg/kg once every 6 weeks.

In an embodiment, the antibody is administered at a dose of 0.1 mg/kg to 100 mg/kg once every 6 weeks. In an embodiment, the antibody is administered at a dose of 0.1 mg/kg to 50 mg/kg once every 6 weeks. In an embodiment, the antibody is administered at a dose of 0.1 mg/kg to 25 mg/kg once every 6 weeks. In an embodiment, the antibody is administered at a dose of 0.1 mg/kg to 20 mg/kg once every 6 weeks. In an embodiment, the antibody is administered at a dose of 0.1 mg/kg, 0.2 mg/kg, 0.3 mg/kg, 0.4 mg/kg, 0.5 mg/kg, 0.75 mg/kg, 1 mg/kg, 2 mg/kg, 3 mg/kg, 4 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 6 weeks. In an embodiment, the antibody is administered at a dose of 0.1 mg/kg, 0.3 mg/kg, 1 mg/kg, 3 mg/kg, 10 mg/kg, or 20 mg/kg once every 6 weeks.

In an embodiment, the antibody is administered at a dose of about 0.1 mg/kg to about 100 mg/kg once every 7 weeks. In an embodiment, the antibody is administered at a dose of about 0.1 mg/kg to about 50 mg/kg once every 7 weeks. In an embodiment, the antibody is administered at a dose of about 0.1 mg/kg to about 25 mg/kg once every 7 weeks. In an embodiment, the antibody is administered at a dose of about 0.1 mg/kg to about 20 mg/kg once every 7 weeks. In an embodiment, the antibody is administered at a dose of about 0.1 mg/kg, about 0.2 mg/kg, about 0.3 mg/kg, about 0.4 mg/kg, about 0.5 mg/kg, about 0.75 mg/kg, about 1 mg/kg, about 2 mg/kg, about 3 mg/kg, about 4 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg once every 7 weeks. In an embodiment, the antibody is administered at a dose of about 0.1 mg/kg, about 0.3 mg/kg, about 1 mg/kg, about 3 mg/kg, about 10 mg/kg, or about 20 mg/kg once every 7 weeks.

In an embodiment, the antibody is administered at a dose of 0.1 mg/kg to 100 mg/kg once every 7 weeks. In an embodiment, the antibody is administered at a dose of 0.1 mg/kg to 50 mg/kg once every 7 weeks. In an embodiment, the antibody is administered at a dose of 0.1 mg/kg to 25 mg/kg once every 7 weeks. In an embodiment, the antibody is administered at a dose of 0.1 mg/kg to 20 mg/kg once every 7 weeks. In an embodiment, the antibody is administered at a dose of 0.1 mg/kg, 0.2 mg/kg, 0.3 mg/kg, 0.4 mg/kg, 0.5 mg/kg, 0.75 mg/kg, 1 mg/kg, 2 mg/kg, 3 mg/kg, 4 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 7 weeks. In an embodiment, the antibody is administered at a dose of 0.1 mg/kg, 0.3 mg/kg, 1 mg/kg, 3 mg/kg, 10 mg/kg, or 20 mg/kg once every 7 weeks.

In an embodiment, the antibody is administered at a dose of about 0.1 mg/kg to about 100 mg/kg once every 8 weeks. In an embodiment, the antibody is administered at a dose of about 0.1 mg/kg to about 50 mg/kg once every 8 weeks. In an embodiment, the antibody is administered at a dose of about 0.1 mg/kg to about 25 mg/kg once every 8 weeks. In an embodiment, the antibody is administered at a dose of about 0.1 mg/kg to about 20 mg/kg once every 8 weeks. In an embodiment, the antibody is administered at a dose of about 0.1 mg/kg, about 0.2 mg/kg, about 0.3 mg/kg, about 0.4 mg/kg, about 0.5 mg/kg, about 0.75 mg/kg, about 1 mg/kg, about 2 mg/kg, about 3 mg/kg, about 4 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg once every 8 weeks. In an embodiment, the antibody is administered at a dose of about 0.1 mg/kg, about 0.3 mg/kg, about 1 mg/kg, about 3 mg/kg, about 10 mg/kg, or about 20 mg/kg once every 8 weeks.

In an embodiment, the antibody is administered at a dose of 0.1 mg/kg to 100 mg/kg once every 8 weeks. In an embodiment, the antibody is administered at a dose of 0.1 mg/kg to 50 mg/kg once every 8 weeks. In an embodiment, the antibody is administered at a dose of 0.1 mg/kg to 25 mg/kg once every 8 weeks. In an embodiment, the antibody is administered at a dose of 0.1 mg/kg to 20 mg/kg once every 8 weeks. In an embodiment, the antibody is administered at a dose of 0.1 mg/kg, 0.2 mg/kg, 0.3 mg/kg, 0.4 mg/kg, 0.5 mg/kg, 0.75 mg/kg, 1 mg/kg, 2 mg/kg, 3 mg/kg, 4 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 8 weeks. In an embodiment, the antibody is administered at a dose of 0.1 mg/kg, 0.3 mg/kg, 1 mg/kg, 3 mg/kg, 10 mg/kg, or 20 mg/kg once every 8 weeks.

In an embodiment, the antibody is administered intravenously at a dose of 0.1 mg/kg once every 3 weeks. In an embodiment, the antibody is administered intravenously at a dose of 0.3 mg/kg once every 3 weeks. In an embodiment, the antibody is administered intravenously at a dose of 1 mg/kg once every 3 weeks. In an embodiment, the antibody is administered intravenously at a dose of 3 mg/kg once every 3 weeks. In an embodiment, the antibody is administered intravenously at a dose of 10 mg/kg once every 3 weeks. In an embodiment, the antibody is administered intravenously at a dose of 20 mg/kg once every 3 weeks.

In an embodiment, before administration of the antibody the subject: a) has confirmed metastatic or advanced, unresectable cancer, measurable disease per Response Evaluation Criteria in Solid Tumors (RECIST) v1.1; b) has a pathologically documented advanced, unresectable, or metastatic cancer that is refractory to or intolerable to standard treatment known to confer benefit or for which no standard treatment is available; c) has an Eastern Cooperative Oncology Group performance status (PS) 0-1; d) has a predicted life expectancy of ≥3 months; e) has one or more of: i) calculated creatinine clearance (CrCL)≥ 50 mL/min by Cockcroft-Gault formula calculation; ii) total bilirubin≤1.5; iii) AST and ALT≤2.5×ULN; iv) hemoglobin≥9.0 g/dL; v) platelets≥100×10⁹ cells/L; or vi) absolute neutrophil count≥1.5×10⁹ cells/L; f) has a Corrected QT interval (QTc)≤470 ms (as calculated by the Fridericia correction formula); and/or g) is not receiving other cancer therapy.

In an embodiment, before administration of the antibody the subject: a) has not received prior treatment with systemic agents comprising radio-immunoconjugates, antibody-drug conjugates, immune/cytokines or monoclonal antibodies within 28 days or five half-lives of the drug, whichever is shorter; b) does not have ongoing toxicity from prior therapy; c) has not undergone a major surgery<3 months prior to administration of the antibody; d) has not received radiation therapy<28 days prior to administration of the antibody; e) has not undergone organ transplantation, allogeneic stem-cell transplantation, or autologous stem-cell transplantation; f) has not received a diagnosis of primary or acquired immunodeficiency; g) has not received treatment with systemic steroids or any other form of immunosuppressive therapy within 14 days prior to administration of the antibody; h) does not have central nervous system (CNS) tumor involvement not definitively treated with surgery or radiation that is active; i) does not have active autoimmune disease requiring immunosuppressive therapy or a history of such disease; j) does not have clinical symptoms of CNS metastases within 28 days prior to administration of the antibody; and/or k) does not have leptomeningeal carcinomatosis.

In an embodiment of the methods described herein, the cancer is associated with elevated DDR1 phosphorylation. Exemplary cancer tissues that may be associated with elevated DDR1 phosphorylation include, but are not limited to, cancer or cancer cells of the bladder, blood, bone, bone marrow, brain, breast, colon, esophagus, intestine, gum, head, kidney, liver, lung, nasopharynx, neck, ovary, prostate, skin, stomach, pancreas, testis, tongue, cervix, or uterus.

Exemplary histological types of cancer that may associated with elevated DDR1 phosphorylation include, but are not limited to, neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; bronchioloalveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non-encapsulating sclerosing carcinoma; adrenal cortical carcinoma; endometrial carcinoma; skin appending carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; bookmark ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma; mixed tumor; Mullerian mixed tumor; nephroblastoma; hepatoblastoma; mesenchymoma, malignant; Brenner tumor, malignant; phyllodes tumor, malignant; synovial sarcoma; mesothelioma, malignant; dysgerminoma; embryonic carcinoma; teratoma, malignant; struma ovarian carcinoma; choriocarcinoma; mesonephroma, malignant; hemangioendothelioma, hemangiopericytoma; chondroblastoma; giant cell tumor of bone; odontogenic tumor, malignant; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; oligodendroglioma; oligodendroblastoma; primitive neuroectodermal; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; small lymphocytic; malignant lymphoma, large cell, diffuse; malignant lymphoma, follicular; mycosis fungoides; other specified non-Hodgkin's lymphomas; malignant histiocytosis; multiple myeloma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; and hairy cell leukemia.

In an embodiment, the cancer is pancreatic cancer, lung cancer, small cell lung cancer, non-small cell lung cancer, colorectal cancer, head and neck cancer, stomach (gastric) cancer, ovarian cancer, breast cancer, kidney cancer, prostate cancer, cervical cancer, brain cancer, skin cancer, melanoma, cholangiocarcinoma, or bone cancer. In an embodiment, the cancer is colorectal cancer, ovarian cancer, or non-small cell lung cancer. In an embodiment, the colorectal cancer is microsatellite stable (MSS). In some embodiments, the cancer is colorectal cancer. In some embodiments, the cancer does not include breast cancer.

In an embodiment, the cancer is not sarcoma, hepatocellular carcinoma, or glioma.

In an embodiment, the cancer expresses DDR1. In an embodiment, the cancer is a solid cancer. In an embodiment, the cancer is a locally advanced or metastatic solid cancer. In an embodiment, the cancer is unresectable.

In an embodiment, the cancer is refractory to immunotherapy. In an embodiment, the cancer is refractory to an antagonist anti-PD-1 antibody, an antagonist anti-PD-L1 antibody, an antagonist anti-PD-L2 antibody, an antagonist anti-CTLA-4 antibody, an antagonist anti-BTLA antibody, an antagonist anti-TREMR antibody, an antagonist anti-TIGIT antibody, an antagonist anti-VISTA antibody, an antagonist anti-TIM-3 antibody, an antagonist anti-LAG-3 antibody, an antagonist anti-CEACAM1 antibody, an agonist anti-GITR antibody, an agonist anti-OX40 antibody, and an agonist anti-CD137 antibody, an agonist anti-DR3 antibody, an agonist anti-TNFSF14 antibody, an agonist anti-CD27 antibody, an agonist anti-ICOS antibody, or an agonist anti-CD28 antibody.

In an embodiment, the subject is not a candidate for standard of care treatment. In an embodiment, the subject is intolerant to a standard of care treatment.

In an embodiment, the cancer is refractory to a standard of care treatment. In an embodiment, the standard of care treatment is chemotherapy and/or radiation. In an embodiment, the standard of care treatment is a chemotherapeutic agent selected from the group consisting of abiraterone acetate, afatinib, aldesleukin, alemtuzumab, alitretinoin, altretamine, amifostine, aminoglutethimide anagrelide, anastrozole, arsenic trioxide, asparaginase, azacitidine, azathioprine, bendamustine, bevacizumab, bexarotene, bicalutamide, bleomycin, bortezomib, busulfan, capecitabine, carboplatin, carmustine, cetuximab, chlorambucil, cisplatin, cladribine, crizotinib, cyclophosphamide, cytarabine, dacarbazine, dactinomycin, dasatinib, daunorubicin, denileukin diftitox, decitabine, docetaxel, dexamethasone, doxifluridine, doxorubicin, epirubicin, epoetin alpha, epothilone, erlotinib, estramustine, entinostat, etoposide, everolimus, exemestane, filgrastim, floxuridine, fludarabine, fluorouracil, fluoxymesterone, flutamide, folate linked alkaloids, gefitinib, gemcitabine, gemtuzumab ozogamicin, GM-CT-01, goserelin, hexamethylmelamine, hydroxyureas, ibritumomab, idarubicin, ifosfamide, imatinib, interferon alpha, interferon beta, irinotecan, ixabepilone, lapatinib, leucovorin, leuprolide, lenalidomide, letrozole, lomustine, mechlorethamine, megestrol, melphalan, mercaptopurine, methotrexate, mitomycin, mitoxantrone, nelarabine, nilotinib, nilutamide, octreotide, ofatumumab, oprelvekin, oxaliplatin, paclitaxel, panitumumab, pemetrexed, pentostatin, polysaccharide galectin inhibitors, procarbazine, raloxifene, retinoic acids, rituximab, romiplostim, sargramostim, sorafenib, streptozocin, sunitinib, tamoxifen, temsirolimus, temozolomide, teniposide, thalidomide, thioguanine, thiotepa, tioguanine, topotecan, toremifene, tositumomab, trametinib, trastuzumab, tretinoin, valrubicin, VEGF inhibitors and traps, vinblastine, vincristine, vindesine, vinorelbine, vintafolide (EC145), and vorinostat.

In an aspect, provided herein is an antibody that specifically binds human DDR1 for use in the treatment of cancer, wherein the treatment is performed according to a method disclosed herein.

In an aspect, provided herein is an antibody that specifically binds human DDR1 for use in the manufacture of a medicament for the treatment of cancer, wherein the treatment is performed according to a method disclosed herein.

### Combination Therapy

In an aspect, provided herein is a method of reducing immune exclusion of a tumor in a subject in need thereof, the method comprising administering to the subject an immune checkpoint inhibitor and an antibody that specifically binds to human DDR1.

In an aspect, provided herein is a method of reducing tumor burden in a subject in need thereof, the method comprising administering to the subject an immune checkpoint inhibitor and an antibody that specifically binds to human DDR1.

In some embodiments, the immune checkpoint inhibitor comprises a PD-1 antagonist. In some embodiments, the PD-1 antagonist is an anti-PD-1 antibody that specifically binds to human PD-1. In some embodiments, the anti-PD-1 antibody comprises pembrolizumab, nivolumab, cemiplimab, sintilimab, penpulimab, tislelizumab, toripalimab, or retifanlimab. In some embodiments, the immune checkpoint inhibitor comprises a PD-L1 antagonist. In some embodiments, the PD-L1 antagonist is an anti-PD-L1 antibody that specifically binds to human PD-L1. In some embodiments, the immune checkpoint inhibitor comprises a PVRIG antagonist. In some embodiments, the PVRIG antagonist is an anti-PVRIG antibody that specifically binds to human PVRIG.

In some embodiments, the methods provided herein comprise administering to a subject a PD-1 or PD-L1 antagonist at a dose of about 100 mg, about 200 mg, about 240 mg, about 350 mg, about 360 mg, about 400 mg, about 480 mg, about 500 mg, about 840 mg, about 1000 mg, about 1200 mg, about 1500 mg, about 1680 mg, about 1700 mg, about 1800 mg, about 1900 mg, or about 2000 mg. In some embodiments, the methods provided herein comprise administering to a subject a PD-1 or PD-L1 antagonist at a dose of about 0.5 mg/kg, about 1 mg/kg, about 2 mg/kg, about 3 mg/kg, about 5 mg/kg, about 10 mg/kg, about 15 mg/kg, about 20 mg/kg, about 25 mg/kg, or about 30 mg/kg.

In some embodiments, the immune checkpoint inhibitor is administered intravenously. In some embodiments, the PD-1 or PD-L1 antagonist is administered intravenously. In some embodiments, the immune checkpoint inhibitor is administered intra-tumoraly. In some embodiments, the PD-1 or PD-L1 antagonist is administered intra-tumoraly. In some embodiments, the PD-1 or PD-L1 antagonist is administered once weekly, every 2 weeks, every 3 weeks, every 4 weeks, every 6 weeks, or every 8 weeks. In some embodiments, the PD-1 or PD-L1 antagonist is administered no more frequently than once weekly, no more frequently than every 2 weeks, no more frequently than every 3 weeks, no more frequently than every 4 weeks, no more frequently than every 6 weeks, or no more frequently than every 8 weeks. In some embodiments, the immune checkpoint inhibitor is administered no more frequently than once weekly, no more frequently than every 2 weeks, no more frequently than every 3 weeks, no more frequently than every 4 weeks, no more frequently than every 6 weeks, or no more frequently than every 8 weeks.

In some embodiments, the PD-1 antagonist is pembrolizumab. In some embodiments, pembrolizumab is administered at a dose of 400 mg once every 6 weeks, or 200 mg once every 3 weeks. In some embodiments, pembrolizumab is administered at a dose of 400 mg once every 3 weeks. In some embodiments, pembrolizumab is administered at a dose of 2 mg/kg once every 3 weeks.

In some embodiments, the PD-1 antagonist is nivolumab. In some embodiments, nivolumab is administered at a dose of 240 mg once every 2 weeks, 360 mg once every 3 weeks, or 480 mg once every 4 weeks. In some embodiments, nivolumab is administered at a dose of 3 mg/kg once every 2 weeks, or 3 mg/kg once every 3 weeks.

In some embodiments, the PD-1 antagonist is cemiplimab. In some embodiments, cemiplimab is administered at a dose of 350 mg once every 3 weeks.

In some embodiments, the PD-1 antagonist is dostarlimab. In some embodiments, dostarlimab is administered at a dose of 500 mg once every 3 weeks, or 1000 mg once every 6 weeks.

In some embodiments, the PD-L1 antagonist is atezolizumab. In some embodiments, atezolizumab is administered at a dose of 840 mg once every 2 weeks, 1200 mg once every 3 weeks, or 1680 mg once every 4 weeks.

In some embodiments, the PD-L1 antagonist is durvalumab. In some embodiments, durvalumab is administered at a dose of 1500 mg once every 3 weeks. In some embodiments, durvalumab is administered at a dose of 10 mg/kg once every 2 weeks, or 20 mg/kg once every 3 weeks.

The antibody that specifically binds to human DDR1 and the immune checkpoint inhibitor can be administered in any suitable manner. In some embodiments, the antibody and the immune checkpoint inhibitor are administered to the subject concurrently, or at the same time, or within the same session (e.g., the same visit to the clinical site). In some embodiments, the antibody and the immune checkpoint inhibitor are administered to the subject in different sessions. The antibody that specifically binds to human DDR1 and the immune checkpoint inhibitor can be administered in any suitable order. In some embodiments, the antibody is administered first, followed by the immune checkpoint inhibitor. In some embodiments, the immune checkpoint inhibitor is administered first, followed by the antibody that specifically binds to human DDR1. In some embodiments, the antibody and the immune checkpoint inhibitor are administered at the same dosing frequency. In some embodiments, the antibody and the immune checkpoint inhibitor are administered at different dosing frequencies. In some embodiments, the antibody is administered more frequently (e.g., by, by about, or by at least 10, 20, 30, 40, 50, 60, 70, 80, 90, 100, 150, 200% more frequently, or more frequently by a percentage in a range defined by any two of the preceding values (e.g., 10-200%, 20-150%, 30-100%, 50-100%, etc.)) than the immune checkpoint inhibitor. In some embodiments, the immune checkpoint inhibitor is administered more frequently (e.g., by, by about, or by at least 10, 20, 30, 40, 50, 60, 70, 80, 90, 100, 150, 200% more frequently, or more frequently by a percentage in a range defined by any two of the preceding values (e.g., 10-200%, 20-150%, 30-100%, 50-100%, etc.)) than the antibody.

In some embodiments, administration of the antibody that specifically binds human DDR1 and the immune checkpoint inhibitor to the subject achieves at least stable disease (e.g., of the cancer). In some embodiments, administration of the antibody that specifically binds human DDR1 and the immune checkpoint inhibitor to the subject prevents further progression of cancer in the subject. In some embodiments, administration of the antibody that specifically binds human DDR1 and the immune checkpoint inhibitor to the subject prevents further growth of tumor size in the subject. In some embodiments, further growth of tumor size is prevented when the tumor size after administering the antibody increases in size by no more than, or no more than about 30%, 25%, 20%, 15%, 10%, 5%, 3%, 1% or no more than a percentage in a range defined by any two of the preceding values (e.g., 1-30%, 3-25%, 5-20%, 1-10%, 1-5%, etc.), or does not increase in size, compared to the size of the tumor at baseline (e.g., before administering the antibody). In some embodiments, administration of the antibody that specifically binds human DDR1 and the immune checkpoint inhibitor to the subject achieves complete response (e.g., elimination of the tumor). In some embodiments, administration of the antibody that specifically binds human DDR1 and the immune checkpoint inhibitor to the subject achieves at least partial response (e.g., shrinking of the tumor by about 50% or more).

### Kits

Also provided herein are kits, e.g., for treating a subject for cancer, where the kits include at least one anti-DDR1 antibody of the present disclosure. In some embodiments, the kit also includes an immune checkpoint inhibitor (e.g., PD-1 or PD-L1 antagonist). In some embodiments, the kit includes one or more containers that holds the anti-DDR1 antibody and/or the immune checkpoint inhibitor. Any suitable container can be used. Suitable containers include without limitation, a vial, test tube, flask, bottle, syringe or other container means, into which the antibody may be placed, or preferably, suitably aliquoted. In some embodiments, the kit includes a pre-fille syringe containing the anti-DDR1 antibody. In some embodiments, the pre-filled syringe is configured to deliver a unit dose (e.g., a therapeutically effective amount) of the anti-DDR1 antibody. In some embodiments, the pre-filled syringe is configured for single-use delivery of a unit dose of the anti-DDR1 antibody. In some embodiments the kit includes instructions for use.

### Additional Embodiments

Additional, non-limiting embodiments, of the present disclosure are provided according to the following numbered arrangements.
1. A method of reducing immune exclusion of a tumor in a subject in need thereof, the method comprising administering to the subject an anti-DDR1 antibody that specifically binds to human DDR1 and an immune checkpoint inhibitor, wherein the anti-DDR1 antibody is administered at a dose of 5 mg to 2000 mg.
2. A method of reducing tumor burden in a subject in need thereof, the method comprising administering to the subject an anti-DDR1 antibody that specifically binds to human DDR1 and an immune checkpoint inhibitor, wherein the anti-DDR1 antibody is administered at a dose of 5 mg to 2000 mg.
3. The method of arrangement 1 or 2, wherein the anti-DDR1 antibody is administered at a dose of about 8 mg to about 1600 mg.
4. The method of arrangement 1 or 2, wherein the anti-DDR1 antibody is administered at a dose of about 8 mg to about 800 mg.
5. The method of any one of arrangements 1-4, wherein the anti-DDR1 antibody is administered at a dose of about 8 mg, about 24 mg, about 80 mg, about 240 mg, about 400 mg, about 800 mg, or about 1600 mg.
6 The method of any one of arrangements 1-5, wherein the anti-DDR1 antibody is administered at a dose of 8 mg, 24 mg, 80 mg, 240 mg, 400 mg, 800 mg, or 1600 mg.
7 The method of any one of arrangements 1-6, wherein the anti-DDR1 antibody is administered intravenously.
8 The method of any one of arrangements 1-7, wherein the anti-DDR1 antibody is administered via intravenous infusion over 60 minutes.
9. The method of any one of arrangements 1-7, wherein the anti-DDR1 antibody is administered via intravenous infusion over 30 minutes.
10. The method of any one of arrangements 1-9, wherein the anti-DDR1 antibody is administered once weekly.
11. The method of any one of arrangements 1-9, wherein the anti-DDR1 antibody is administered once every 2 weeks.
12. The method of any one of arrangements 1-9, wherein the anti-DDR1 antibody is administered once every 3 weeks.
13. The method of any one of arrangements 1-9, wherein the anti-DDR1 antibody is administered once every 4 weeks.
14. The method of any one of arrangements 1-9, wherein the anti-DDR1 antibody is administered once every 8 weeks.
15. The method of any one of arrangements 1-9, wherein the anti-DDR1 antibody is administered intravenously at a dose of 8 mg once every 3 weeks.
16. The method of any one of arrangements 1-9, wherein the anti-DDR1 antibody is administered intravenously at a dose of 24 mg once every 3 weeks.
17. The method of any one of arrangements 1-9, wherein the anti-DDR1 antibody is administered intravenously at a dose of 80 mg once every 3 weeks.
18. The method of any one of arrangements 1-9, wherein the anti-DDR1 antibody is administered intravenously at a dose of 240 mg once every 3 weeks.
19. The method of any one of arrangements 1-9, wherein the anti-DDR1 antibody is administered intravenously at a dose of 400 mg once every 3 weeks.
20. The method of any one of arrangements 1-9, wherein the anti-DDR1 antibody is administered intravenously at a dose of 800 mg once every 3 weeks.
21. The method of any one of arrangements 1-9, wherein the anti-DDR1 antibody is administered intravenously at a dose of 1600 mg once every 3 weeks.
22. The method of any one of arrangements 1-21, wherein the immune checkpoint inhibitor comprises a PD-1 or PD-L1 antagonist.
23. The method of any one of arrangements 1-22, wherein the PD-1 or PD-L1 antagonist is administered at a dose of 100 mg to 2000 mg.
24. The method of any one of arrangements 1-22, wherein the PD-1 or PD-L1 antagonist is administered at a dose of 200 mg, 240 mg, 350 mg, 360 mg, 400 mg, 480 mg, 500 mg, 840 mg, 1000 mg, 1200 mg, 1500 mg, or 1680 mg.
25. The method of any one of arrangements 1-22, wherein the PD-1 or PD-L1 antagonist is administered at a dose of 0.5 mg/kg to 30 mg/kg.
26. The method of any one of arrangements 1-22, wherein the PD-1 or PD-L1 antagonist is administered at a dose of 1 mg/kg, 2 mg/kg, 3 mg/kg, 10 mg/kg, or 20 mg/kg.
27. The method of any one of arrangements 1-26, wherein the PD-1 or PD-L1 antagonist is administered intravenously.
28. The method of any one of arrangements 1-27, wherein the PD-1 or PD-L1 antagonist is administered once weekly.
29. The method of any one of arrangements 1-27, wherein the PD-1 or PD-L1 antagonist is administered once every 2 weeks.
30. The method of any one of arrangements 1-27, wherein the PD-1 or PD-L1 antagonist is administered once every 3 weeks.
31. The method of any one of arrangements 1-27, wherein the PD-1 or PD-L1 antagonist is administered once every 4 weeks.
32. The method of any one of arrangements 1-27, wherein the PD-1 or PD-L1 antagonist is administered once every 6 weeks.
33. The method of any one of arrangements 1-27, wherein the PD-1 or PD-L1 antagonist is administered once every 8 weeks.
34. The method of any one of the preceding arrangements, wherein the subject has cancer.
35. The method of arrangement 34, wherein the administration of the anti-DDR1 antibody and the PD-1 or PD-L1 antagonist treats the cancer in the subject.
36. The method of arrangement 34, wherein the PD-1 antagonist is an anti-PD-1 antibody that specifically binds to human PD-1.
37. The method of arrangement 34, wherein the PD-L1 antagonist is an anti-PD-L1 antibody that specifically binds to human PD-L1.
38. The method of arrangement 36, wherein the anti-PD-1 antibody is pembrolizumab, nivolumab, dostarlimab, cemiplimab, sintilimab, penpulimab, tislelizumab, toripalimab, or retifanlimab.
39. The method of arrangement 37, wherein the anti-PD-L1 antibody is avelumab, atezolizumab, or durvalumab.
40. The method of any one of arrangements 1-36, wherein the PD-1 antagonist is pembrolizumab.
41. The method of arrangement 40, wherein pembrolizumab is administered at a dose of 400 mg once every 6 weeks.
42. The method of arrangement 40, wherein pembrolizumab is administered at a dose of 200 mg once every 3 weeks.
43. The method of arrangement 40, wherein pembrolizumab is administered at a dose of 2 mg/kg once every 3 weeks.
44. The method of any one of arrangements 1-36, wherein the PD-1 antagonist is nivolumab.
45. The method of arrangement 44, wherein nivolumab is administered at a dose of 240 mg once every 2 weeks.
46. The method of arrangement 44, wherein nivolumab is administered at a dose of 360 mg once every 3 weeks.
47. The method of arrangement 44, wherein nivolumab is administered at a dose of 480 mg once every 4 weeks.
48. The method of arrangement 44, wherein nivolumab is administered at a dose of 3 mg/kg once every 2 weeks.
49. The method of arrangement 44, wherein nivolumab is administered at a dose of 3 mg/kg once every 3 weeks.
50. The method of any one of arrangements 1-36, wherein the PD-1 antagonist is cemiplimab.
51. The method of arrangement 50, wherein cemiplimab is administered at a dose of 350 mg once every 3 weeks.
52. The method of any one of arrangements 1-36, wherein the PD-1 antagonist is dostarlimab.
53. The method of arrangement 52, wherein dostarlimab is administered at a dose of 500 mg once every 3 weeks.
54. The method of arrangement 52, wherein dostarlimab is administered at a dose of 1000 mg once every 6 weeks.
55. The method of any one of arrangements 1-35, or 37, wherein the PD-L1 antagonist is atezolizumab.
56. The method of arrangement 55, wherein atezolizumab is administered at a dose of 840 mg once every 2 weeks.
57. The method of arrangement 55, wherein atezolizumab is administered at a dose of 1200 mg once every 3 weeks.
58. The method of arrangement 55, wherein atezolizumab is administered at a dose of 1680 mg once every 4 weeks.
59. The method of any one of arrangements 1-35, or 37, wherein the PD-L1 antagonist is durvalumab.
60. The method of arrangement 59, wherein durvalumab is administered at a dose of 10 mg/kg once every 2 weeks.
61. The method of arrangement 59, wherein durvalumab is administered at a dose of 20 mg/kg once every 3 weeks.
62. The method of arrangement 59, wherein durvalumab is administered at a dose of 1500 mg once every 3 weeks.
63. The method of any one of the preceding arrangements, wherein the cancer expresses DDR1.
64. The method of any one of the preceding arrangements, wherein the cancer is a solid cancer.
65. The method of any one of the preceding arrangements, wherein the cancer is a locally advanced or metastatic solid cancer.
66. The method of any one of the preceding arrangements, wherein the cancer is unresectable.
67. The method of any one of the preceding arrangements, wherein the cancer is refractory to immunotherapy.
68. The method of arrangement 67, wherein the immunotherapy is an antagonist anti-PD-1 antibody, an antagonist anti-PD-L1 antibody, an antagonist anti-PD-L2 antibody, an antagonist anti-PD-1/anti-PD-L1 antibody bispecific antibody, an antagonist anti-CTLA-4 antibody, an antagonist anti-BTLA antibody, an antagonist anti-TREMR antibody, an antagonist anti-TIGIT antibody, an antagonist anti-VISTA antibody, an antagonist anti-TIM-3 antibody, an antagonist anti-LAG-3 antibody, an antagonist anti-CEACAM1 antibody, an agonist anti-GITR antibody, an agonist anti-OX40 antibody, and an agonist anti-CD137 antibody, an agonist anti-DR3 antibody, an agonist anti-TNFSF14 antibody, an agonist anti-CD27 antibody, an agonist anti-ICOS antibody, or an agonist anti-CD28 antibody.
69. The method of arrangement 67, wherein the immunotherapy is an antagonist anti-PD-L1 antibody.
70. The method of any one of the preceding arrangements, wherein the cancer is not sarcoma, hepatocellular carcinoma, or glioma.
71. The method of any one of the preceding arrangements, wherein the cancer is pancreatic cancer, lung cancer, small cell lung cancer, non-small cell lung cancer, colorectal cancer, head and neck cancer, stomach (gastric) cancer, ovarian cancer, breast cancer, kidney cancer, prostate cancer, cervical cancer, brain cancer, skin cancer, melanoma, cholangiocarcinoma, or bone cancer.
72. The method of any one of the preceding arrangements, wherein the cancer is colorectal cancer, ovarian cancer, or non-small cell lung cancer.
73. The method of any one of the preceding arrangements, wherein the subject is not a candidate for standard of care treatment.
74. The method of any one of the preceding arrangements, wherein the cancer is refractory to a standard of care treatment.
75. The method of arrangement 74, wherein the standard of care treatment is chemotherapy or radiation.
76. The method of any one of arrangements 1-75, wherein administration of the anti-DDR1 antibody and the PD-1 or PD-L1 antagonist reduces tumor size in the subject.
77. The method of any one of the preceding arrangements, wherein before administration of the anti-DDR1 antibody and the PD-1 or PD-L1 antagonist the subject:
   a) has confirmed metastatic or advanced, unresectable cancer, measurable disease per Response Evaluation Criteria in Solid Tumors (RECIST) v1.1;
   b) has a pathologically documented advanced, unresectable, or metastatic cancer that is refractory to or intolerable to standard treatment known to confer benefit or for which no standard treatment is available;
   c) has an Eastern Cooperative Oncology Group performance status (PS) 0-1;
   d) has a predicted life expectancy of ≥3 months;
   e) has one or more of:
      i) calculated creatinine clearance (CrCL)≥50 mL/min by Cockcroft-Gault formula calculation;
      ii) total bilirubin≤1.5;
      iii) AST and ALT≤2.5×ULN;
      iv) hemoglobin≥9.0 g/dL;
      v) platelets≥100× 10⁹ cells/L; or
      vi) absolute neutrophil count≥1.5×10⁹ cells/L;
   f) has a Corrected QT interval (QTc)≤470 ms (as calculated by the Fridericia correction formula); and/or
   g) is not receiving other cancer therapy.
78. The method of any one of the preceding arrangements, wherein before administration of the anti-DDR1 antibody and the PD-1 or PD-L1 antagonist the subject:
   a) has not received prior treatment with systemic agents comprising radio-immunoconjugates, antibody-drug conjugates, immune/cytokines or monoclonal antibodies within 28 days or five half-lives of the drug, whichever is shorter;
   b) does not have ongoing toxicity from prior therapy;
   c) has not undergone a major surgery<3 months prior to administration of the anti-DDR1 antibody;
   d) has not received radiation therapy<28 days prior to administration of the anti-DDR1 antibody;
   e) has not undergone organ transplantation, allogeneic stem-cell transplantation, or autologous stem-cell transplantation;
   f) has not received a diagnosis of primary or acquired immunodeficiency;
   g) has not received treatment with systemic steroids or any other form of immunosuppressive therapy within 14 days prior to administration of the anti-DDR1 antibody;
   h) does not have central nervous system (CNS) tumor involvement not definitively treated with surgery or radiation that is active;
   i) does not have active autoimmune disease requiring immunosuppressive therapy or a history of such disease;
   j) does not have clinical symptoms of CNS metastases within 28 days prior to administration of the anti-DDR1 antibody; and/or
   k) does not have leptomeningeal carcinomatosis.
79. The method of any one of the preceding arrangements, wherein the anti-DDR1 antibody comprises: a heavy chain variable region (VH) comprising the CDRH1, CDRH2, and CDRH3 amino acid sequences of the VH amino acid sequence set forth in SEQ ID NO: 7, or a variant thereof comprising 1-5 amino acid changes in any one of the CDRH1, CDRH2, or CDRH3 amino acid sequences; and/or a light chain variable region (VL) comprising the CDRL1, CDRL2, and CDRL3 amino acid sequences of the VL amino acid sequence set forth in SEQ ID NO: 8 or 9, or a variant thereof comprising 1-5 amino acid changes in any one of the CDRL1, CDRL2, or CDRL3 amino acid sequences.
80. The method of arrangement 79, wherein:
(a) the VH comprises the CDRH1, CDRH2, and CDRH3 amino acid sequences, respectively, of:
   SEQ ID NO: 1, or a variant thereof comprising 1-5 amino acid changes,
   SEQ ID NO: 2, or a variant thereof comprising 1-5 amino acid changes, and
   SEQ ID NO: 3, or a variant thereof comprising 1-5 amino acid changes; and/or
(b) the VL comprises the CDRL1, CDRL2, and CDRL3 amino acid sequences, respectively, of:
   SEQ ID NO: 4, or a variant thereof comprising 1-5 amino acid changes,
   SEQ ID NO: 5, or a variant thereof comprising 1-5 amino acid changes, and
   SEQ ID NO: 6, or a variant thereof comprising 1-5 amino acid changes.
81. The method of arrangement 79 or 80, wherein the anti-DDR1 antibody that specifically binds to human DDR1 comprises the CDRH1, CDRH2, CDRH3, CDRL1, CDRL2, and CDRL3 amino acid sequences set forth in SEQ ID NOs: 1, 2, 3, 4, 5, and 6, respectively.
82. The method of any one of arrangements 79-81, wherein the anti-DDR1 antibody that specifically binds to human DDR1 comprises: a VH comprising an amino acid sequence which is at least 75%, 80%, 85%, 90%, 95%, 96%, 97%, 98%, 99%, or 100% identical to the amino acid sequence set forth in SEQ ID NO: 7; and/or a VL comprising an amino acid sequence which is at least 75%, 80%, 85%, 90%, 95%, 96%, 97%, 98%, 99%, or 100% identical to the amino acid sequence set forth in SEQ ID NO: 8 or 9.
83. The method of arrangement 82, wherein the anti-DDR1 antibody comprises a VH comprising the amino acid sequence set forth in SEQ ID NO: 7 and a VL comprising the amino acid sequence set forth in SEQ ID NO: 8.
84. The method of arrangement 82, wherein the anti-DDR1 antibody comprises a VH comprising the amino acid sequence set forth in SEQ ID NO: 7 and a VL comprising the amino acid sequence set forth in SEQ ID NO: 9.
85. The method of any one of arrangements 79-84, wherein the anti-DDR1 antibody comprises a heavy chain comprising an amino acid sequence which is at least 75%, 80%, 85%, 90%, 95%, 96%, 97%, 98%, 99%, or 100% identical to the amino acid sequence set forth in SEQ ID NO: 10 or 11 and/or a light chain comprising an amino acid sequence which is at least 75%, 80%, 85%, 90%, 95%, 96%, 97%, 98%, 99%, or 100% identical to the amino acid sequence set forth in SEQ ID NO: 12.
86. The method of arrangement 85, wherein the anti-DDR1 antibody comprises a heavy chain comprising the amino acid sequence set forth in SEQ ID NO: 10 and a light chain comprising the amino acid sequence set forth in SEQ ID NO: 12.
87. The method of arrangement 85, wherein the anti-DDR1 antibody comprises a heavy chain comprising the amino acid sequence set forth in SEQ ID NO: 11 and a light chain comprising the amino acid sequence set forth in SEQ ID NO: 12.
88. The method of any one of the preceding arrangements, wherein the anti-DDR1 is administered to the subject before the PD-1 or PD-L1 antagonist.
89. The method of any one of the preceding arrangements, wherein the anti-DDR1 antibody is administered to the subject after the PD-1 or PD-L1 antagonist.
90. The method of any one of the preceding arrangements, wherein the anti-DDR1 antibody is administered to the subject at the same time as the PD-1 or PD-L1 antagonist.
91. An anti-DDR1 antibody and a PD-1 or PD-L1 antagonist for use in the treatment of cancer, wherein the treatment is performed according to the method of any one of the previous arrangements.
92. An anti-DDR1 antibody and a PD-1 or PD-L1 antagonist for use in the manufacture of a medicament for the treatment of cancer, wherein the treatment is performed according to the method of any one of the previous arrangements.
93. Use of an anti-DDR1 antibody and a PD-1 or PD-L1 antagonist for the treatment of cancer, wherein the treatment is performed according to the method of any one of the previous arrangements.
94. A therapeutic combination comprising an antibody that specifically binds to human DDR1 and a PD-1 or PD-L1 antagonist.
95. A combination comprising an anti-DDR1 antibody and a PD-1 or PD-L1 antagonist for use in the treatment of cancer, wherein the treatment is performed according to the method of any one of the previous arrangements.

### EXAMPLES

### Example 1—Anti-DDR1 Antibody 9H-1 Binding Characteristics and Anti-Tumor Activity

Anti-DDR1 antibody 9H-1 was tested for its binding characteristics in various species and for its ability to inhibit tumor growth in vivo in a mouse model of cancer.

### A. Binding Characteristics

First, 9H-1 was tested for its affinity to DDR1 protein from human, mouse, rat, and cynomolgus monkey. Biacore assays were used to determine the affinity of 9H-1 for the DDR1 extracellular domain from human, mouse, rat, and cynomolgus monkey. Table 5 below shows that 9H-1 binds to DDR1 from various species with a high affinity.

TABLE 5
Affinity of 9H-1 for DDR1 from Different Species

| | K_{D} | K_{D} | kₐ | k_{d} | t_{1/2} |
|---|---|---|---|---|---|
| Antigen | (nM) | (ng/mL) | (M⁻¹s⁻¹) | (s⁻¹) | (s) |
| Human | 1.36 | 204 | 1.33 × 10⁵ | 1.81 × 10⁻⁴ | 3.84 × 10³ |
| DDR1 | | | | | |
| Mouse | 16.91 | 2,537 | 2.35 × 10⁵ | 3.97 × 10⁻³ | 174 |
| DDR1 | | | | | |
| Rat DDR1 | 2.28 | 342 | 1.99 × 10⁵ | 4.55 × 10⁻⁴ | 1.52 × 10³ |
| Cyno DDR1 | 1.30 | 195 | 1.16 × 10⁵ | 1.50 × 10⁻⁴ | 4.62 × 10³ |

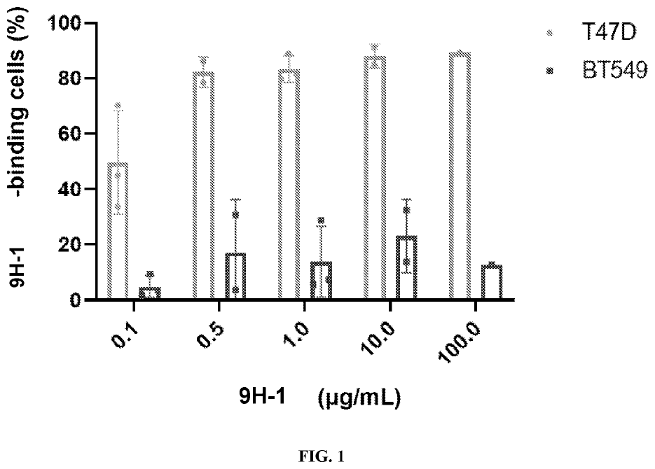

Next, 9H-1 was tested for its ability to bind human cells that express DDR1. A flow cytometry analysis showed that 9H-1 binds significantly more T47D cells (DDR1 high/DDR2 low) compared to BT549 cells (DDR1 low/DDR2 high) (see FIG. 1).

### B. Anti-Tumor Activity

Briefly, mice were inoculated with human DDR1-expressing E0771 mouse Ddr1−/− cells (an engineered triple negative breast cancer cell line). The mice were treated with 10 mg/kg 9H-1, 9H-1-LALAPG (which includes a heavy chain of SEQ ID NO:11 with the following mutations: L238A, L239A, P333G, numbered according to SEQ ID NO:11), or an IgG control every other day. Tumors were measured bi-weekly.

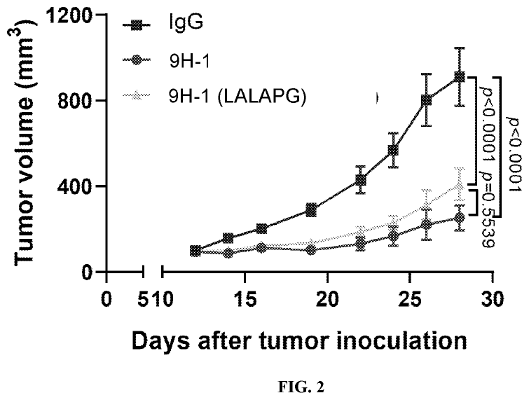

The results shown in FIG. 2 demonstrate that 9H-1 significantly inhibits tumor growth in mice compared to the mice treated with the IgG control.

### Example 2—Anti-DDR1 Antibody 9H-1 In Vivo Pharmacokinetics to Model FIH Doses

In order to determine first in human (FIH) doses for anti-DDR1 antibody 9H-1, single doses of the antibody were administered to cynomolgus monkeys for pharmacokinetic modeling.

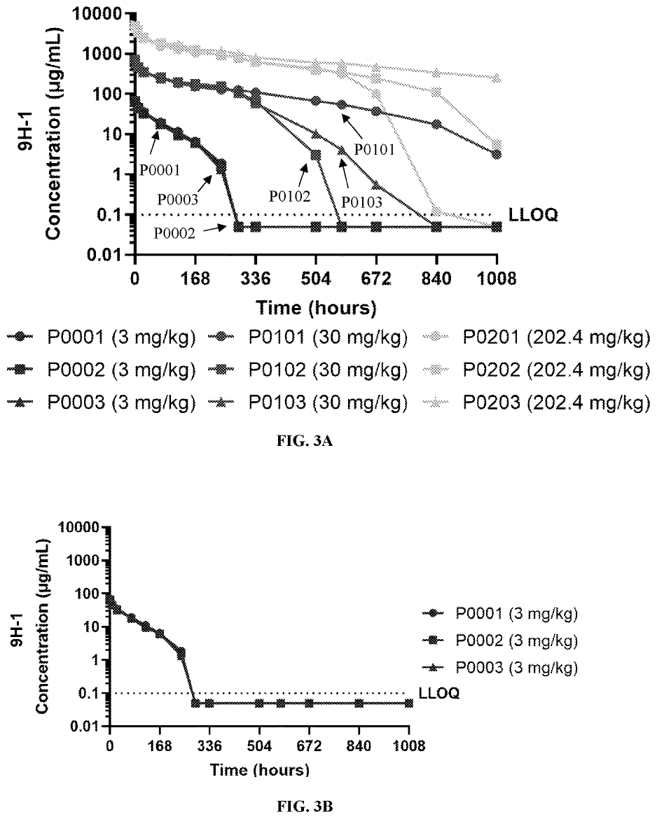

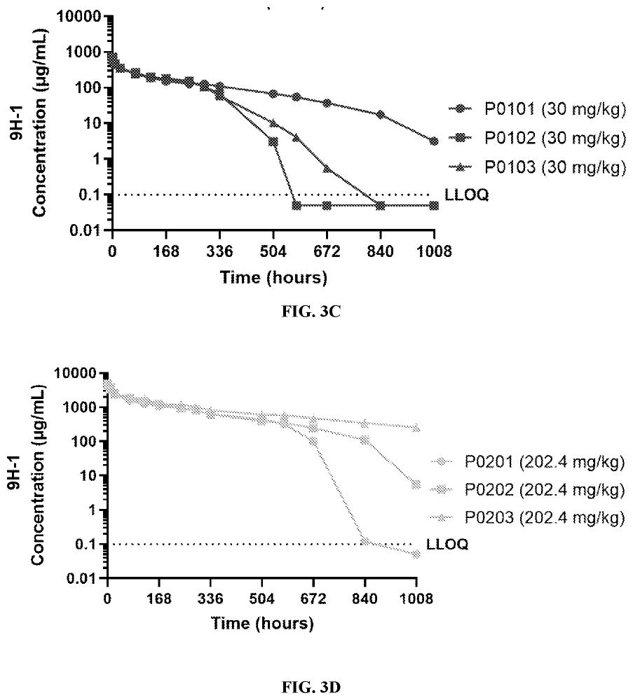

The 9H-1 antibody was administered to cynomolgus monkeys by intravenous infusion pump at a low, intermediate, and high dose, according to Table 6 below. Serum samples were collected pre-dose and at 0.25, 8, 24, 72, 120, 168, 240, 288, 336, 504, 576, 672, 840, and 1008 hours. Analysis was done by antigen capture ELISA, the individual concentrations of 9H-1 in each animal is shown in FIG. 3A-D.

TABLE 6
Dose Levels of 9H-1 in Cynomolgus Monkeys

| | Dose | Dose | Dose | Number of |
|---|---|---|---|---|
| | Level | Concentration | Volume | Animals |
| Group | (mg/kg) | (mg/mL) | (mL/kg) | (male) |
| 1 (Low) | 3 | 0.375 | 8 | 3 |
| 2 | 30 | 3.75 | 8 | 3 |
| (Intermediate) | | | | |
| 3 (High) | 202.4 | 25.3 | 8 | 3 |

A 2-compartment model with first-order elimination was selected to fit PK data from the cynomolgus monkey study. During model building, a 3-compartment structure resulted in slightly lower AIC, however, the 2-compartment model was selected to reduce the risk of overparameterization.

The 3-compartment model goodness of fit is included for comparison. Parameters were scaled using the following equations:

$$V h = tvV * ( BWh / BWm ) \hat{} 1$$

$$V 2 h - tvV 2 * ( BWh / BWm ) \hat{} 1$$

$$Clh - tvCl * ( BWh / BWm ) \hat{} 0.8$$

$$Cl 2 h = tvCl 2 * ( BWh / BWm ) \hat{} 0.8$$

The average weight of the cynomolgus monkeys was 2.15 kg, while 80 kg was used as an estimate for human weight. Some animals and timepoints were excluded from the modeling database due to apparent irregular PK profiles indicative of antidrug antibodies. The following animals and timepoints were included in the modeling:
P0101: 0.25-240 hours
P0102: 0.25-240 hours
P0103: 0.25-240 hours
P0201: 0.25-504 hours
P0202: 0.25-504 hours
P0203: 0.25-1008 hours

Table 7 below shows the scaled human model parameters:

TABLE 7
Scaled Human Model Parameters

| Parameter | Estimate | |
|---|---|---|
| V | 3707.94 | (mL) |
| V2 | 3794.43 | (mL) |
| Cl | 10.85 | (mL/hr) |
| Cl2 | 50.5 | (mL/hr) |

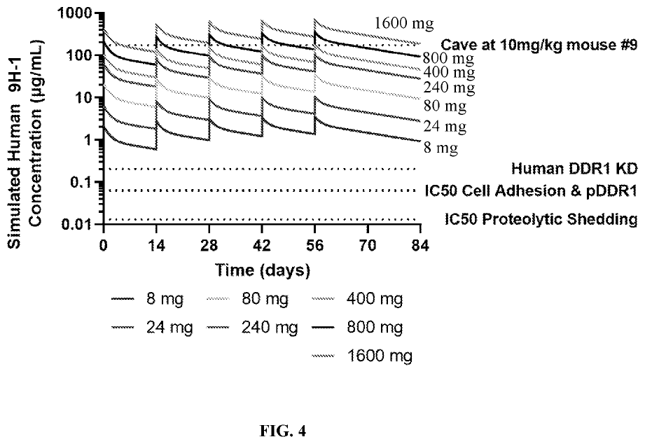

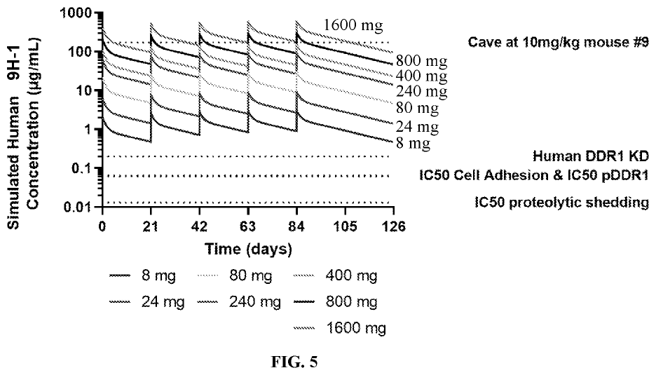

Based on modeling of the cynomolgus data and the scaled human parameters, FIG. 4 and FIG. 5 show the simulated concentrations of 9H-1 at fixed doses of 8-1600 mg, administered once every 2 weeks (Q2W, FIG. 4) or once every 3 weeks (Q3W, FIG. 5). These data show that the concentration of 9H-1 remains above the concentration sufficient to bind human DDR1 and inhibit DDR1 function, when the antibody is administered at any of the fixed doses either Q2W or Q3W. Table 8 below steady state parameters of simulated human 9H-1 exposure.

TABLE 8
Simulated Human 9H-1 Pharmacokinetics

| Dose | Dose | Cₘₐₓ | Tₘₐₓ | Half-Life | AUC₀₋₃₃₆ | AUC₀₋₅₀₄ | AUC_{inf} |
|---|---|---|---|---|---|---|---|
| Number | (mg) | (μg/mL) | (hr) | (hr) | (hr * μg/mL) | (hr * μg/mL) | (hr * μg/mL) |
| Dose 20 | 8 | 3.784 | 0.083 | 507.1 | 737.3 | — | 1929 |
| Q2W | 24 | 11.352 | 0.083 | 507.1 | 2211.9 | — | 5787 |
| | 80 | 37.84 | 0.083 | 507.1 | 7373 | — | 19290 |
| | 240 | 113.52 | 0.083 | 507.1 | 22119 | — | 57870 |
| | 400 | 189.2 | 0.083 | 507.1 | 36865 | — | 96450 |
| | 800 | 378.4 | 0.083 | 507.1 | 73730 | — | 192900 |
| | 1600 | 756.8 | 0.083 | 507.1 | 147460 | — | 385800 |
| Dose 20 | 8 | 3.113 | 0.083 | 507.1 | — | 736 | 1438 |
| Q3W | 24 | 9.339 | 0.083 | 507.1 | — | 2208 | 4314 |
| | 80 | 31.13 | 0.083 | 507.1 | — | 7360 | 14380 |
| | 240 | 93.39 | 0.083 | 507.1 | — | 22080 | 43140 |
| | 400 | 155.65 | 0.083 | 507.1 | — | 36800 | 71900 |
| | 800 | 311.3 | 0.083 | 507.1 | — | 73600 | 143800 |
| | 1600 | 622.6 | 0.083 | 507.1 | — | 147200 | 287600 |

### Example 3—Investigation of the Safety, Pharmacokinetics, Pharmacodynamics, and Activity of 9H-1 and Anti-PD-1 Antibody Combination Therapy in Adult Subjects with Locally Advanced or Metastatic Solid Tumors

This example describes an open-label Phase 1 dose escalation and expansion trial to evaluate the safety and tolerability, efficacy, pharmacokinetics (PK), pharmacodynamics (PD), and activity of dosage regimens of 9H-1 (an anti-DDR1 humanized monoclonal antibody) in combination with pembrolizumab (an anti-PD-1 antibody) in adults with advanced solid cancers. 9H-1 is a first-in-class, humanized monoclonal antibody targeting DDR1 that is designed to disrupt the collagen alignment in the tumor stroma and allow a subject's own immune cells to infiltrate the tumor.

DDR1 targeted therapy has the potential to benefit broad number of cancer subjects. Analysis of tumor biopsies from a variety of cancer types has demonstrated some measurable level of DDR1 expression across a range of malignancies, with some notable exceptions. DDR1 expression is noted to be low in hepatocellular carcinoma and sarcomas, which is why they are excluded from enrollment in the trial. To understand the level of DDR1 expression that is associated with benefit and better understand the cancer types that may potentially benefit, the enrollment in the trial includes all solid tumors with the exceptions of hepatocellular carcinoma and sarcoma, as discussed above, as well as gliomas due to uncertainty about penetration of the blood-brain barrier.

### A. Study Design

### Overall Design

The objective of the dose escalation plan is to determine the safety, tolerability, optimal dose, and optimal dosing schedule of 9H-1 in combination with an anti-PD-1 antibody therapy. An additional expansion trial of cohorts of subjects with specific histology, treatment history, and/or baseline characteristics will be conducted to evaluate for signals of antitumor activity.

For each combination cohort, initially 3 subjects will be enrolled. The first combination cohort will begin at dose level 3 and no earlier than after the next higher dose monotherapy cohort has been cleared to escalate in the BOIN methodology and by the SRC in the Phase 1 study described in Example 3. Pembrolizumab will be given at 400 mg per dosing event. The study will include five cohorts of subjects, 9H-1 will be administered intravenously to the subjects at doses of 8 mg (cohort 1), 24 mg (cohort 2), 80 mg (cohort 3), 240 mg (cohort 4), 800 mg (cohort 5), or 1600 mg (cohort 6) every three weeks. The subjects enrolled in cohorts 1 and 2 can have their doses escalated to the dose level of cohort 3 if they have not had grade≥2 related adverse events after 90 days of treatment and dose level 3 has not been eliminated per the dose escalation design described below.

9H-1 will be administered as a diluted solution, intravenously (IV) over 1 hour, using an intravenous line containing a sterile, non-pyrogenic, low-protein binding 0.2 micron to 5 micron in-line or add-on filter. After the first two doses have been given to each subject without any evidence of infusion reaction, the infusion time can be decreased to 30 minutes with permission of the site PI. The SRC may adjust pre-medications and infusion duration if infusion reactions are observed. Pembrolizumab will be administered in a diluted solution intravenously over 30 minutes through an intravenous line containing a sterile, non-pyrogenic, low-protein binding 0.2 micron to 5 micron in-line or add-on filter. No other drugs will be administered through the same infusion line.

Starting with dose cohort 4, up to ten biomarker backfill slots will be made available to subjects wishing to enroll in the trial. Backfill spots will be made available after the dosing cohort has been completely enrolled and been deemed safe to continue escalation or determined to be the recommended phase 2 dose (RP2D). Pre-treatment archival tissue or fresh pre-treatment biopsies and a post-treatment biopsy at six weeks after initiation of treatment will be mandatory. Pre- and post-treatment CD8 PET scans will be mandatory if the capability is available at the enrolling site. A specific number of slots available for biomarker backfill and the specific histology types that will be included or excluded will be determined based on available data.

### Concomitant Medication

Any medication or vaccine (including over-the-counter (OTC) or prescription medicines, vitamins, and/or herbal supplements) that the participant is receiving at the time of enrollment or receives during the study must be recorded along with reason for use, dates of administration, including start and end dates, and dosage information, including dose and frequency.

The Medical Monitor should be contacted if there are any questions regarding concomitant or prior therapy. Medications or vaccinations specifically prohibited in the exclusion criteria are not allowed during the ongoing trial. If there is a clinical indication for any medication or vaccination specifically prohibited during the trial, discontinuation from study treatment or vaccination may be required. The investigator is to discuss prohibited medication and vaccination with the Sponsor's Clinical Director. The final decision on any supportive therapy or vaccination rests with the investigator and/or the subject's primary physician. However, the decision to continue the subject on study treatment requires the mutual agreement of the investigator, the Sponsor, and the subject.

The following medications and vaccinations are prohibited during the study:
Antineoplastic systemic chemotherapy or biological therapy
Immunotherapy not specified in this protocol
Chemotherapy not specified in this protocol
Investigational agents other than 9H-1
Radiation therapy (radiation therapy to a symptomatic solitary lesion or to the brain may be allowed at the investigator's discretion)
Live or live attenuated vaccines within 30 days prior to the first dose of study treatment and while participating in the study (killed vaccines are allowed). Note: Any licensed COVID-19 vaccine (including for Emergency Use) in a particular country is allowed in the study as long as they are mRNA vaccines, adenoviral vaccines, or inactivated vaccines. These vaccines will be treated just as any other concomitant therapy. Investigational vaccines (i.e., those not licensed or approved for Emergency Use) are not allowed.
Systemic glucocorticoids except when used for the following purposes:
   To modulate symptoms of an AE that is suspected to have an immunologic etiology
   For the prevention of emesis
   To premedicate for IV contrast allergies
   To treat COPD exacerbations (only short-term oral or IV use in doses>10 mg/day prednisone equivalent)
   For chronic systemic replacement not to exceed 10 mg/day prednisone equivalent
   Other glucocorticoid use except when used for the following purposes:
      For topical use or ocular use
      Intraarticular joint use
      For inhalation in the management of asthma or chronic obstructive pulmonary disease
      Note: Inhaled steroids are allowed for management of asthma.

If the investigator determines that a subject requires any of the aforementioned treatments for any reason, 9H-1 must be discontinued.

All treatments that the investigator considers necessary for a subject's welfare may be administered at the discretion of the investigator in keeping with the community standards of medical care.

All concomitant medication will be recorded on the eCRF, including all prescription, over-the-counter products, herbal supplements, and IV medications and fluids. If changes occur during the study period, documentation of drug dosage, frequency, route, and date should also be included on the eCRF.

All concomitant medications received within 28 days prior to the first dose of study treatment and up to 30 days after the last dose of study treatment should be recorded. All concomitant medications administered during SAEs or ECIs are to be recorded.

### BOIN Design for Dose Escalation

The Bayesian optimal interval (BOIN) design will be used to find the maximum tolerated dose (MTD). The BOIN design is implemented in a simple way similar to the traditional 3+3 design but is more flexible and possesses superior operating characteristics that are comparable to those of the more complex model-based designs, such as the continual reassessment method (CRM). Dose-limiting toxicities (DLTs) that occur within the first cycle will be used for dose finding.

The steps to implement the BOIN design are described as follows:
1. Perform accelerated titration up to dose level 3 (1 mg/kg) as follows: treat the first subject at dose level 1. If no DLT is observed, escalate the dose to the next higher level. Continue this one-subject-per-dose dose escalation process until either of the following events is observed: (i) the first instance of DLT, or (ii) the second instance of moderate (grade 2) toxicity. Then treat two additional subjects at the current dose level. Hereafter, subjects are treated in cohorts of size 3 as described in steps 2 and 3. In the case that the titration reaches dose level 3 without observing (i) or (ii), subjects are treated in cohorts of size 3 from dose level 4.
2. To assign a dose to the next cohort of subjects, conduct dose escalation/de-escalation according to the rule displayed in Table 9 below. When using Table 9, please note the following:
3. “Eliminate” means eliminate the current and higher doses from the trial to prevent treating any future subjects at these doses because they are overly toxic.
4. When we eliminate a dose, automatically de-escalate the dose to the next lower level. When the lowest dose is eliminated, stop the trial for safety. In this case, no dose should be selected as the MTD.
5. If none of the actions (i.e., escalation, de-escalation, or elimination) is triggered, treat the new subjects at the current dose.
6. If the current dose is the lowest dose and the rule indicates dose de-escalation, treat the new subjects at the lowest dose unless the number of DLTs reaches the elimination boundary, at which point terminate the trial for safety.
7. If the current dose is the highest dose and the rule indicates dose escalation, treat the new subjects at the highest dose.
8. Repeat step 2 until the maximum sample size of 30 is reached or stop the trial if the number of evaluable subjects treated at the current dose reaches 9 and the decision according to Table 9 is to stay at the current dose.

TABLE 9
Dose escalation/de-escalation rule for the BOIN design.

| | Number of evaluable subjects | | | | | | | | | | | |
|---|---|---|---|---|---|---|---|---|---|---|---|---|
| | treated at current dose | | | | | | | | | | | |
| | 1 | 2 | 3 | 4 | 5 | 6 | 7 | 8 | 9 | 10 | 11 | 12 |
| Escalate if # of | 0 | 0 | 0 | 0 | 1 | 1 | 1 | 1 | 2 | 2 | 2 | 2 |
| DLT ≤ | | | | | | | | | | | | |
| De-escalate if # | 1 | 1 | 2 | 2 | 2 | 3 | 3 | 3 | 4 | 4 | 4 | 5 |
| of DLT ≥ | | | | | | | | | | | | |
| Eliminate if # | NA | NA | 3 | 3 | 4 | 4 | 5 | 5 | 5 | 6 | 6 | 7 |
| of DLT ≥ | | | | | | | | | | | | |

“# of DLT” is the number of subjects with at least 1 DLT. When none of the actions (i.e., escalate, de-escalate. or eliminate) is triggered, stay at the current dose for treating the next cohort of subjects.
“NA” means that a dose cannot be eliminated before treating 3 DLT-evaluable subjects.

All adverse events (AEs) specified in Table 10 below should count as DLTs except those that are clearly due to disease progression or extraneous causes.

TABLE 10
Adverse events.
Hematologic

| | |
|---|---|
| Neutropenia | Grade ≥4 |
| Febrile Neutropenia | Any Grade |
| Thrombocytopenia | Grade ≥3 that is associated with bleeding event or a Grade 4 |
| | thrombocytopenia |

Non-Hematologic Toxicities
Any Grade 3 adverse events with the following exceptions:
Grade 3 nausea, vomiting, or diarrhea lasting less than 72 hours
Grade 3 fatigue <1 week
Grade ≥3 electrolyte abnormality that lasts <72 hours, unless the subject has clinical
symptoms, in which case the electrolyte abnormality should count as a DLT.
Grade 3 amylase or lipase elevation NOT associated with symptoms or clinical
manifestations of pancreatitis.
For subjects with hepatic metastases, AST or ALT > 8 × ULN or AST or ALT > 5 × ULN
for ≥14 days (unless clearly due to disease progression)
A dose delay for greater than 14 days due to an AE.
Immune-Mediated Toxicities
Any Grade immune-related encephalopathy or myelitis
Grade ≥3 immune mediated pneumonitis, colitis, hepatitis, ocular toxicities, or neuropathy
Any Grade ≥3 endocrinopathy that does not resolve with treatment or is not adequately
controlled by hormone replacement.
Grade 2 hepatitis that persists for >12 weeks
Any Grade 4 immune-related adverse event
Any other Grade ≥3 immune-related adverse events that do not resolve to ≤Grade 1 or to
baseline with immunosuppressive therapy within 21 days, with the exception of alopecia and
vitiligo.
Any ≥Grade 3 immune-related AE not included above that in the opinion of the
Investigator is assessed to be dose limiting.
Other Toxicities
Any Grade 5 AE not clearly due to disease progression or extraneous causes.
Any AE that in the opinion of the Investigator is assessed to be dose limiting.

### Long Term Extension Phase

Subjects will receive treatment with 9H-1 and pembrolizumab until meeting criteria for study discontinuation. Subjects in the dose escalation portions of the trial will be able to transition from their assigned dose cohort to receive the RP2D, once that is determined.

### Study Termination

The end of the study is defined as the primary completion date, which is defined as the date on which the last subject completes the last visit (phone contact is also considered as a visit) or when the Sponsor decides to terminate the study, whichever occurs first.

Recruitment will cease when one of the following occurs:
Study treatment is considered too toxic to continue treatment before the anticipated number of subjects are recruited. This assessment will be made by the CTSC.
The stated number of subjects to be recruited is reached. This number may be increased to include replacement subjects for those who are not DLT-evaluable and subjects added to intermediate dose or expanded cohorts.
The stated objectives of the study are achieved.
Sponsor decision.

In terminating the study, the Sponsor and the Investigators must ensure that adequate consideration is given to the protection of the subjects' interests.

A subject is considered to have completed the study if he or she has completed all phases of the study, including the Follow-Up Visit 30 Days (+7 days) after their last dose of study treatment received unless they are experiencing ongoing study treatment-related AEs or SAEs. For subjects being followed for ongoing SAEs or study treatment-related AEs, follow-up visits will continue at least every 4 weeks until resolution or return to baseline, stabilization of the event, the subject is lost to follow-up or withdraws consent, or the Medical Monitor deems it necessary, whichever occurs first. If a subject begins another anti-cancer therapy, Safety Follow-up Visits will stop.

The total length of the study, from screening of the first participant to the end of the study, is expected to be approximately 40 months.

### B. Study Population

### Inclusion Criteria

Participants are eligible for study inclusion if the following criteria apply:
1. Be willing and able to read, understand, and sign an Informed Consent Form.
2. Be age≥18 years.
3. Biopsy-confirmed metastatic or advanced, unresectable malignancy, measurable disease per Response Evaluation Criteria in Solid Tumors (RECIST) v1.1 as assessed at Screening, excluding sarcomas and gliomas.
4. Has a pathologically documented advanced/unresectable or metastatic cancer that is refractory to or intolerable to standard treatment known to confer benefit or for which no standard treatment is available.
5. Subject must have an Eastern Cooperative Oncology Group performance status (PS) 0-1.
6. Subject must have a predicted life expectancy of ≥3 months.
7. Subject must have the following laboratory values (obtained≤28 days prior to enrollment)
   a. Calculated creatinine clearance (CrCL) must be ≥50 mL/min by Cockcroft-Gault formula calculation.
   b. Total bilirubin≤1.5×ULN unless has known history of Gilbert's syndrome (in which case, total bilirubin must be ≤3×ULN).
   c. AST and ALT≤2.5×ULN.
   d. Hemoglobin≥9.0 g/dL.
   e. Platelets≥100×10⁹ cells/L.
   f. Absolute neutrophil count≥1.5×10⁹ cells/L (without the use of hematopoietic growth factors).
8. Corrected QT interval (QTc)≤470 ms (as calculated by the Fridericia correction formula).
9. Women of child-bearing potential (WOCBP) must have a negative urine pregnancy test within 72 hours prior to first administration of 9H-1 and a negative urine pregnancy test on the first day of dosing.
10. WOCBP and males with female partners of child-bearing potential must agree to use adequate birth control throughout their participation and for 90 days following the last dose of 9H-1.
11. Subject must be willing to adhere to the study visit schedule and the prohibitions and restrictions specified in this protocol.
12. Subject must have a site of disease amenable to biopsy and be a candidate for tumor biopsy according to the treating institution's guidelines or have archived tissue available at enrollment.
   a. Subjects with sites of disease not amenable to biopsy may be considered after discussion with the Sponsor.
13. The subject is not enrolled in any other clinical trial and is not receiving other therapy directed at their malignancy.
14. Subjects may have received prior PD-1/PD-L1 therapy, but no prior history of immune-related adverse events to immune checkpoint inhibitors≥grade 3. For patients with grade 2 immune-related adverse events with prior immune checkpoint inhibitors, these must have resolved to grade 1 at the time of enrollment with the following exceptions:
   a. Alopecia and vitiligo,
   b. Grade 2 neuropathy that is stable,
   c. Well-controlled hypo/hyperthyroidism or other endocrinopathies that are well controlled with hormone replacement.
   Such exceptions must be assessed by the Investigator (and approved by the Sponsor) as not placing the subject at undue safety risk from participating in this study.

Additional Inclusion criteria for biomarker backfill subjects:
1. Subject must have a site of disease amenable to biopsy and be a candidate for tumor biopsy according to the treating institution's guidelines. Subjects must be willing to undergo a new tumor biopsy or have archived tissue available at enrollment and be willing to undergo at least one biopsy while on study.
   a. Subjects with sites of disease not amenable to biopsy may be considered after discussion with the Sponsor.

Additional inclusion criteria for expansion trial:
1. Subjects with following indications can be included in the expansion trial:
   a. Cohort B: Colon cancer, MSS, without BRAFV600E mutation that has been previously treated or intolerant of with 5-fluorouracil, oxaliplatin, irinotecan and appropriate biologic therapy.
   b. Cohort C: Platinum-resistant ovarian carcinoma, with one or more previous lines of therapy, with platinum-resistance defined as a recurrence or progression within 6 months of completing the 1st or subsequent course of platinum therapy, TMB-low
   c. Cohort D: NSCLC with primary resistance to PD-1/PD-L1 therapy, defined as progression within 6 months of initiating therapy with a PD-1 or PD-L1 therapy, 1 or more prior therapies, and no history of targetable driver mutation(s).

### Exclusion Criteria

Participants are excluded from the trial if any of the following criteria apply:
1. Subject has received prior treatment with systemic agents, including, but not limited to, radio-immunoconjugates, antibody-drug conjugates, immune/cytokines and monoclonal antibodies (e.g., anti-CTLA4, anti-PD-1 and anti-PD-L1) within 28 days or five half-lives of the drug, whichever is shorter
2. Subject has ongoing toxicity from prior therapy>Grade 1 according to the CTCAE, with the following
   a. alopecia, and vitiligo
   b. Grade≤2 neuropathy
   c. Well-controlled hypo/hyperthyroidism or other endocrinopathies that are well controlled with hormone replacement
3. Subject has undergone a major surgery (excluding minor procedures e.g., placement of vascular access)<3 months prior to administration of 9H-1,
4. Subject has received radiation therapy<28 days prior to administration of 9H-1.
   a. Exception: limited (e.g., pain palliation) radiation therapy is allowed prior to and during study treatment as long as there are no acute toxicité subject has measurable disease outside the radiation field.
5. Subject has undergone or is anticipated to undergo organ transplantation, including allogeneic or autologous stem-cell transplantation, at any time.
6. Subject has a diagnosis of immunodeficiency, either primary or acquired.
7. Subject has received treatment with systemic steroids or any other form of immunosuppressive therapy within 14 days prior to administration of 9H-1. Exception: inhaled or topical (to include mouthwash) steroids and adrenal replacement doses are permitted in the absence of active autoimmune disease.
8. Subject has an active or prior history of autoimmune disease requiring immunosuppressive therapy. Exceptions can be made.
9. Subject has a known severe intolerance to or hypersensitivity reactions to monoclonal antibodies, Fc-bearing proteins (e.g., soluble receptors or other Fc fusion proteins), or IV immunoglobulin preparations; prior history of human anti-human antibody response; known allergy to any of the study medications, their analogues, or excipients in the various formulations of any agent.
10. Subject has central nervous system (CNS) tumor involvement not definitively treated with surgery or radiation that is active (including evidence of cerebral edema by magnetic resonance imaging (MRI), or progression from prior imaging study), or has had any requirement for steroids, or clinical symptoms of/from CNS metastases within 28 days prior to study treatment.
11. The subject has leptomeningeal carcinomatosis, regardless of treatment history.
12. Subject has current second malignancy at other sites (exceptions: nonmelanomatous skin cancer, adequately treated in situ carcinoma (e.g., cervical), or indolent prostate cancer under observation). A history of other malignancies is allowed as long as subject has been free of recurrence for ≥2 years, or if the subject has been treated with curative intent within the past 2 years and, in the opinion of the Investigator, is unlikely to have a recurrence.
13. Subject has active and clinically significant bacterial, fungal, or viral infection, including known Hepatitis A, B, or C or HIV (testing not required).
14. Subject has received live vaccines within the past 30 days (inactivated vaccines are allowed; seasonal vaccines should be up to date>30 days prior to administration of 9H-1).
15. Women who are pregnant or breastfeeding.
16. History of any of the following≤6 months before first dose:
   a. congestive heart failure New York Heart Association Grade III or IV,
   b. unstable angina,
   c. myocardial infarction,
   d. unstable symptomatic ischemic heart disease,
   e. uncontrolled hypertension despite appropriate medical therapy,
   f. ongoing symptomatic cardiac arrhythmias of >Grade 2,
   g. pulmonary embolism or symptomatic cerebrovascular events, or any other serious cardiac condition (e.g., pericardial effusion or restrictive cardiomyopathy). Chronic atrial fibrillation on stable anticoagulant therapy is allowed.
17. Subject has any contraindications to the imaging assessments or other study procedures that subjects will be undergoing.
18. Subject has any medical or social condition that, in the opinion of the Investigator, might place a subject at increased risk, affect compliance, or confound safety or other clinical study data interpretation.

### C. Efficacy Assessments

Participants will undergo tumor assessments, until loss of clinical benefit, as determined by the Investigator (unless the participant withdraws consent, or the Sponsor terminates the study). All participants who discontinue study intervention for reasons other than disease progression (e.g., adverse events) will continue tumor assessments until death, disease progression, initiation of another systemic anticancer therapy, loss to follow-up, withdrawal of consent, or study termination, whichever occurs first. At the Investigator's discretion, tumor assessments may be repeated at any time if progressive disease is suspected.

Measurable and evaluable lesions should be assessed and documented at screening. Tumor assessments performed as standard of care prior to obtaining informed consent and within 30 days prior to enrollment do not have to be repeated at screening.

Screening assessments must include CT scans (with IV contrast unless contraindicated and oral contrast as appropriate per institutional standards) of the chest/abdomen and pelvis. If a CT scan with contrast is contraindicated (i.e., in participants with contrast allergy or impaired renal clearance), a non-contrast CT scan of the chest may be performed and MRI scans of the abdomen, pelvis should be performed. MRIs of the brain will be required for all subjects with neurologic symptoms.

If a CT scan for a tumor assessment is performed in a positron emission tomography (PET)/CT scanner, the CT acquisition must be consistent with the standards for a full contrast diagnostic CT scan.

Bone scans (technetium-99m [TC-99m]) or sodium fluoride (NaF) PET should be performed at screening if clinically indicated. If bone metastases are present at screen and cannot be seen on CT or MRI scans, or if clinically indicated, TC-99m and NaF-PET bone scan should be repeated when complete response is identified in target disease or when progression in bone is suspected.

CT scans of the neck or extremities should also be performed if clinically indicated and repeated throughout the study if there is evidence of disease at screening.

CD8 PET scans obtained during the study are considered exploratory and will not be used to assess treatment response by RECIST v1.1. However, the CD8 PET scans should be performed with a CT scan consistent with the standards for a full contrast diagnostic CT scan, when it is possible. The contrasted CT scan should be evaluated separate from the CD8 PET scan and will count as an efficacy endpoint evaluation.

All measurable and evaluable lesions should be reassessed at each subsequent tumor evaluation. The same radiographic procedures used to assess disease sites at screening should be used for subsequent tumor assessments (e.g., same contrast protocol for CT scans).

Response will be assessed by the Investigator using RECIST v1.1. Assessments should be performed by the same evaluator, if possible, to ensure internal consistency across visits. Results must be reviewed by the Investigator before dosing at the next cycle.

The Overall Response Rate (ORR) is defined as the primary efficacy endpoint for each dose expansion cohort. A subject will be considered as a responder if a CR/PR is observed from the overall objective response assessments, by Investigator(s) per RECIST. This endpoint will be analyzed following the BOP2 method. At the end of the cohort, the ORR will be calculated with the 95% confidence interval using binomial exact method for each expansion cohort separately, where the denominator will include all efficacy evaluable subjects of the cohort (who either completed at least three post-baseline RECIST assessments or discontinued the study because of death, AE, lack of efficacy, or progressive disease). As exploratory endpoints, overall confirmed response rate (a CR/PR confirmed by the next RECIST assessment), ORR per iRECIST, and ORR, including all dosed subjects in the denominator (ITT approach) will be analyzed similarly with the 95% confidence interval for each expansion cohort after enrollment is complete.

Progression Free Survival (PFS) is defined as an exploratory efficacy endpoint for each histology-specific expansion cohort, which is the duration from Day 1 to the date of first disease progression, as assessed by Investigator per RECIST v1.1 (or as primary reason of discontinuation), or death. This time-to-event variable will be censored at the date of last RECIST assessment, or the date of discontinuation not because of disease progression or death. This endpoint will be analyzed using Kaplan-Meier method for each expansion cohort, including all dosed subjects. PFS according to iRECIST criteria will be derived and analyzed similarly.

Duration of Response (DOR) is defined as an exploratory for each histology-specific expansion cohort, which is the duration from first observed response (CR/PR per RECIST v1.1) to the date of first disease progression after the response. This time-to-event variable will be censored at the date of last RECIST assessment, or date of discontinuation not because of reason of AEs, disease progression, or death, after the initial response date. This endpoint will be analyzed using Kaplan-Meier method for each expansion cohort, including all responders of the cohort. DOR according to iRECIST criteria will be derived and analyzed similarly.

Disease Control Rate (DCR) is defined as the percentage of subjects with advanced or metastatic cancer who have achieved complete response, partial response, and stable disease by RECIST v1.1. This endpoint will be analyzed using Kaplan-Meier method for each expansion cohort, including all dosed subjects.

Overall Survival (OS) is defined as an exploratory endpoint for each histology-specific expansion cohort, which is the duration from Day 1 to the date of death. This time-to-event variable will be censored at the date of last known alive. This endpoint will be analyzed using Kaplan-Meier method for each expansion cohort, including all dosed subjects.

### D. Safety Assessments

Safety assessments will consist of monitoring and recording adverse events, including serious adverse events (SAEs) and adverse events (AEs) of special interest, performing protocol-specified safety laboratory assessments, measuring protocol specify vital signs, and conducting other protocol specified test that are deemed critical to the safety evaluation of the study.

### E. Pharmacokinetics

Serum PK for 9H-1 and pembrolizumab will be collected in all parts of the study. Planned PK timepoints may be updated or discontinued after initial assessment of the PK profile has been characterized. If emerging PK data show that a less frequent schedule of events is warranted, PK sampling may be reduced without a protocol amendment.

The following PK parameters will be determined for 9H-1 using non-compartmental methods: Cₘₐₓ, time to Cₘₐₓ (Tₘₐₓ), last validated plasma concentration (Cₗₐₛₜ), AUC₀₋ₗₐₛₜ (AUC_{504 h} or AUC_{336 h} or AUC_{672 h}-Cycle 1 Day 1 and Cycle 3 Day 1 doses), time to last measurable concentration (Tₗₐₛₜ), (t_{1/2}), accumulation ratio of 9H-1 and pembrolizumab, and, if possible, Vd and CL. Possible relationships between PK and PD variables, efficacy, and/or selected toxicities will be explored, as appropriate.

PK profiles to assess PK properties of 9H-1 and pembrolizumab will be collected from all enrolled subjects. All subjects enrolled in the combination dose escalation study will have a full PK profile collected. Subjects enrolled in the expansion study will have an abbreviated PK sampling schedule.

Residual PK, PD, and ADA samples used for PK and ADA analysis may also be used for exploratory PK and/or PD analyses related to 9H-1 therapy and cancer. This could include using leftover samples for exploratory, alternative PK assay development and analysis.

9H-1 levels will be determined using blood samples collected before and after dosing through the EOT Visit. These determinations will be used to calculate the single- and repeat-dose PK profiles for each evaluable subject at each dose level administered. 9H-1 in combination with pembrolizumab, and pembrolizumab alone PK parameters will be estimated using non-compartmental analysis. PK parameters will include, but are not limited to, accumulation ratio, Cₘₐₓ, Tₘₐₓ, Cₗₐₛₜ, Tₗₐₛₜ, AUC₀₋ₗₐₛₜ (AUC_{504 h} or AUC_{336 h} or AUC_{672 h}), Vd, CL, and t_{1/2}.

The 9H-1 and pembrolizumab concentrations will be listed and summarized in tabular formats using descriptive statistics. 9H-1 and pembrolizumab concentrations will be plotted against timepoints by cohort. Individual and summary PK parameters will be listed and summarized in tabular format using descriptive statistics.

### F. Immunogenicity

The relationship between immunogenicity and 9H-1 serum concentration and adverse events will be represented graphically and tabulated to characterize a relationship between the changes in ADA at screening and serum concentration of 9H-1 in combination with pembrolizumab.

In addition, the potential correlation between immunogenicity and other endpoints (major safety, efficacy, and biomarker parameters) may be evaluated. This will be done in two steps. First, a descriptive analysis will be performed graphically between immunogenicity change from screening values and major safety, efficacy, and biomarker parameters (either as categories or continuous variables). If any potential correlation is identified, further investigation will be performed using a mechanism-based modeling approach, as appropriate.

The concentration/adverse event-immunogenicity relationship will be represented graphically and tabulated to characterize a relationship between the changes from screening immunogenicity presence and serum concentration of single agent 9H-1.

In addition, the potential correlation between immunogenicity and other endpoints (major safety, efficacy, and biomarker parameters) may be evaluated. This will be done in two steps. First, a descriptive analysis will be performed graphically between immunogenicity change from screening values and major safety, efficacy, and biomarker parameters (either as categories or continuous variables). If any potential correlation is identified, further investigation will be performed using a mechanism-based modeling approach, as appropriate.

### G Biomarkers

The exploratory biomarker objectives of this study are to identify biomarkers associated with immuno-oncology study intervention by assessing tumor tissue and circulating soluble factors, including, but not limited to, DNA, RNA, enzymes, growth factors, cytokines, antibodies, and immune cells in tissue and blood. Additionally, microbiome profiles may be evaluated from stool samples. Evaluation of baseline levels and/or changes with study intervention may be performed to determine association with clinical outcomes, including clinical response and resistance, as well as study intervention tolerability.

Collection of samples for biomarker research is part of this study. The following samples for biomarker research are required and will be collected from all participants in this study as specified in the SoA:
Blood, including PBMC
Skin biopsies
Tumor tissue biopsy (before, archival, or fresh)
Optional samples for biomarker research that should be collected from participants in the study where possible are the following:
   Tumor tissue biopsy (six weeks post-treatment), optional for all subjects. Mandatory for biomarker backfill subjects.

Samples may be tested for genetic analysis on tumor and blood samples, including, but not limited to, assays on circulating free DNA, DNA from tumor and/or immune cells and T cell receptor sequencing may be performed. This research may evaluate whether genetic variation corresponds with the outcomes of treatment. If genetic variation is found to predict efficacy or adverse events, the data might inform optimal use of therapies in cancer subjects. Circulating soluble analytes may be assessed that may include, but not limited to, immune cytokines, growth factors, antibodies, and/or markers associated with immune characteristics and activation or cancer. Additionally, tumor and blood samples will be collected before and on study intervention for immune cell profiling that may include immune cell phenotyping, enumerations and/or activation state. Both genome-wide and targeted messenger RNA (mRNA) expression profiling and sequencing in tumor and/or blood may be performed to define gene signatures that correlate with treatment outcomes. Epigenetic analyses may also be performed as these are important biomarkers for some cancers.

Blood, skin, tissue, and tumor biopsies will be collected from subjects throughout the trial for biomarker analysis.

### H. Genetics

Sample collection, storage, and shipment instructions for planned genetic analysis samples will be provided in the Laboratory Manual. Samples should be collected for planned analysis of associations between genetic variants in germline/tumor DNA and clinical outcomes. Blood for planned genetic analysis will be collected for DNA as described in the Schedule of Activities. If a documented law or regulation prohibits (or local IRB/IEC does not approve) sample collection for these purposes, then such samples should not be collected at the corresponding sites. Additional DNA extracted from planned genetic analysis samples will be stored for future biomedical research only if participant signs the Future Biomedical Research consent.

In the event of DNA extraction failure, a replacement genetic blood sample may be requested from the participant. Signed informed consent will be required to obtain a replacement sample unless it was included in the original consent.

### I. Objectives and Endpoints

TABLE 11
Objectives and endpoints.

| Objectives | Endpoints |
|---|---|
| Primary | |
| Evaluate the safety and tolerability of 9H-1 | Safety: incidence and severity of adverse |
| in combination with pembrolizumab | events and serious adverse events, including |
| Determine the Recommended phase 2 dose | changes in laboratory parameters, vital |
| [RP2D] of 9H-1 together with | signs, and ECGs |
| pembrolizumab | Tolerability: incidence of dose interruptions |
| Determine the optimal dosing interval of | and dose reductions |
| 9H-1. | Incidence of DLTs/AEs/SAEs during the |
| Define PK profile of 9H-1 together with | first cycle of treatment with 9H-1 |
| pembrolizumab | Serum PK parameters (e.g., Cₘₐₓ, Tₘₐₓ, |
| | AUCs, clearance, half-lives, V_{d}); serum |
| | concentration vs time profiles |
| Secondary | |
| Determine degree of target engagement by | Incidence of ADA |
| 9H-1 at different dose levels | |
| Determine the presence of antidrug | |
| antibodies (ADA) and their impact on the | |
| PK profile of 9H-1 in combination with | |
| pembrolizumab antibody therapy | |
| Evaluate the PK of pembrolizumab | |
| Evaluate the incidence and persistance of | |
| (ADA) formation against anti- | |
| pembrolizumab and its impact on the PK | |
| profile of pembrolizumab when in | |
| combination with 9H-1 antibody therapy | |
| Exploratory | |
| Evaluate anti-tumor activity of 9H-1 together | Associations between expression of DDR1 |
| with pembrolizumab | and other immunological markers such as, |
| Determine pharmacodynamic effects of 9H- | but not restricted to, PD-L1, CD8 tumor |
| 1 combination with pembrolizumab in the | infiltrating lymphocyte (TIL) level |
| skin and tumor microenvironment and their | TIL counts and expression of immune- |
| relationships to dose | related genes (RNA/protein) at baseline and |
| Evaluate tumor immune biomarkers and | after treatment |
| their association with treatment outcome | Circulating collagen fragments at baseline |
| Identify molecular biomarkers that may be | and after treatment |
| indicative of clinical response/resistance, | Collagen alignment in the tumor bed at |
| safety, pharmacodynamic activity, and/or the | baseline and after treatment |
| mechanism of action of 9H-1 together with | CD8 PET scan intensity in and around the |
| pembrolizumab | tumor at baseline and after treatment |
| | Changes in phosphorylated DDR1 in skin |
| | and tumor tissue |
| | Target engagement in skin and tumor tissue |
| | Change in circulating DDR1 Extracellular |
| | Domain (ECD) levels |
| | Objective response rate (ORR) |
| | Disease Control Rate (DCR) |
| | Progression free survival (PFS) |
| | Overall survival (OS) |
| | Duration of response (DOR) |

TABLE 12
Objectives and endpoints for expansion study.

| Objectives | Endpoints |
|---|---|
| Primary | |
| Evaluate anti-tumor activity of 9H-1 as | Objective Response Rate by RECIST v1.1 |
| monotherapy and in combination with | Safety: incidence and severity of study- |
| pembrolizumab in selected indications | related adverse events and serious adverse |
| Evaluate the safety and tolerability of 9H-1 in | events |
| combination with pembrolizumab | Tolerability: incidence of dose interruptions |
| | and dose reductions |
| Secondary | |
| N/A | ORR by iRECIST |
| | DCR |
| | PFS |
| | DOR |
| Exploratory | |
| To determine the pharmacodynamic effects | Associations between expression of DDR1 |
| of 9H-1 in combination with pembrolizumab | and other immunological markers such as, |
| in the skin and tumor microenvironment and | but not restricted to, PD-L1, CD8 tumor |
| in circulation | infiltrating lymphocyte (TIL) level |
| To identify biomarkers that may be indicative | Changes in immune contexture, including |
| of clinical response/resistance, safety, | spatial distribution, and expression |
| pharmacodynamic activity, and/or the | (RNA/protein) in tumor tissue at baseline and |
| mechanism of action of 9H-1 together with | after treatment |
| pembrolizumab | Circulating collagen fragments at baseline |
| | and after treatment |
| | Collagen alignment in the tumor bed at |
| | baseline and after treatment |
| | CD8 PET scan intensity in and around the |
| | tumor and in reference normal tissues at |
| | baseline and after treatment |
| | Changes in circulating DDR1 Extracellular |
| | Domain (ECD) levels and target engagement |
| | in serum |

For purposes of analysis, the following populations are defined:

TABLE 13
Populations for analysis.

| Population | Description |
|---|---|
| Enrolled | All participants who sign the ICF |
| DLT- | All enrolled subjects who complete at least 3 weeks of |
| Evaluable | follow-up after a dose of 9H-1 |
| Clinically | All enrolled subjects who complete at least one follow-up |
| Evaluable | cross-sectional image to assess response |
| Safety | All participants who take at least 1 dose 9H-1 |

### J. Statistical Analyses

The study will use an adaptive approach, with on-treatment data guiding trial adaptation and success or futility of each combination in each study subpopulation.

The statistical analysis plan will be developed and finalized before database lock and will describe the study populations to be included in the analyses, and procedures for accounting for missing, unused, and spurious data. This section is a summary of the planned statistical analyses of the primary and secondary endpoints.

### Example 4—Anti-DDR1 Antibody Treatment Enhances T Cell Infiltration Induced by Anti PD-1 Antibody

This example demonstrates that a rabbit monoclonal antibody version of 9H-1 (an anti-DDR1 humanized monoclonal antibody) (where the rabbit monoclonal antibody includes a heavy chain and light chain as shown in Table 17 below) enhances T cell infiltration into solid tumors in mice when administered in combination with an anti-PD-1 antibody.

TABLE 17

| Rabbit monoclonal | | |
|---|---|---|
| antibody version | | SEQ |
| of 9H-1 | Sequence | ID NO |
| Light chain | ELVMTQTPASVEAAVGGTVTIKCQASQSIGSVLA | 15 |
| | WYQQKPGQRPKLLISGVFDLASGVPSRFKGSGSGT | |
| | EFTLTISDLECADAATYYCQYIPYGSSPFGGGTEVV | |
| | VKGDPVAPTVLIFPPAADQVATGTVTIVCVANKYF | |
| | PDVTVTWEVDGTTQTTGIENSKTPQNSADCTYNLS | |
| | STLTLTSTQYNSHKEYTCKVTQGTTSVVQSFNRGD | |
| | C | |
| Heavy chain | QSLEESGGRLVTPGTPLTLTCTASGFSLNRYYMLW | 16 |
| | VRQAPGEGLEWIGTISYGDTTYYASWAKGRFTISK | |
| | TSTTVDLKMTSPTTEDTATYFCARADTGDNGYLG | |
| | LQLWGPGTLVTVSSGQPKAPSVFPLAPCCGDTPSS | |
| | TVTLGCLVKGYLPEPVTVTWNSGTLTNGVRTFPSV | |
| | RQSSGLYSLSSVVSVTSSSQPVTCNVAHPATNTKV | |
| | DKTVAPSTCSKPTCPPPELLGGPSVFIFPPKPKDTL | |
| | MISRTPEVTCVVVDVSQDDPEVQFTWYINNEQVR | |
| | TARPPLREQQFNSTIRVVSTLPIAHQDWLRGKEFK | |
| | CKVHNKALPAPIEKTISKARGQPLEPKVYTMGPPR | |
| | EELSSRSVSLTCMINGFYPSDISVEWEKNGKAEDN | |
| | YKTTPAVLDSDGSYFLYSKLSVPTSEWQRGDVFTC | |
| | SVMHEALHNHYTQKSISRSPGK | |

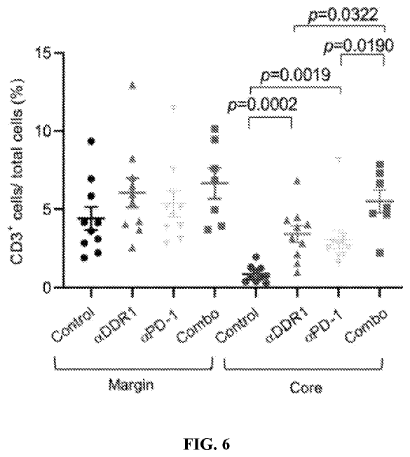

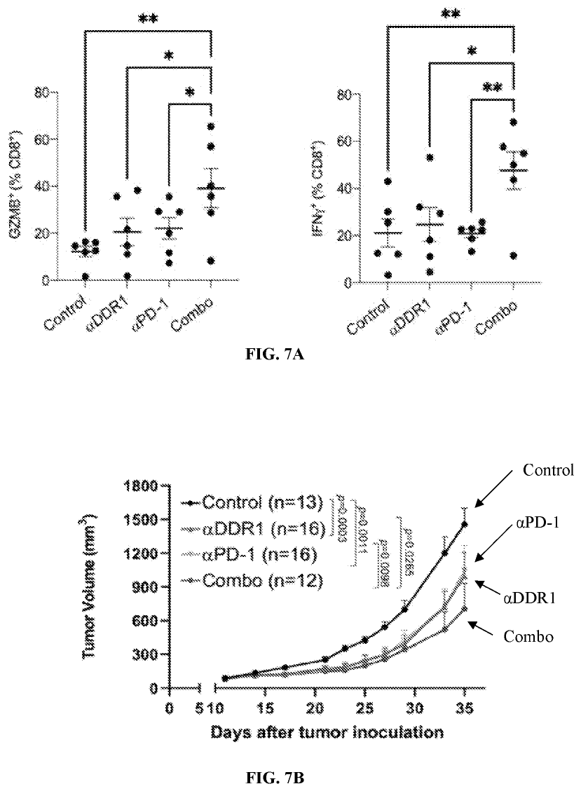

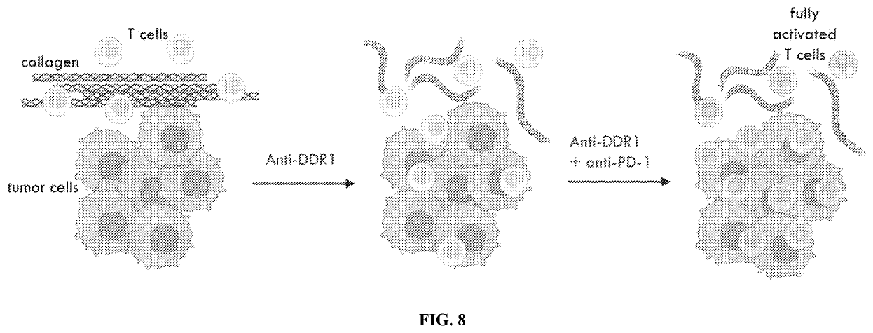

As shown in FIG. 6, the percentage of CD3+ cells in the core of solid tumors in mice was significantly higher when the rabbit monoclonal antibody version of 9H-1 (αDDR1) was administered in combination with mouse anti-PD-1 antibody (αPD-1), as compared to either the rabbit monoclonal antibody version of 9H-1 or mouse anti-PD-1 antibody alone. Moreover, FIG. 7A and FIG. 7B demonstrates significantly increased T cell activation and significantly reduced tumor volume, respectively, for the combination therapy, as compared to either monotherapy or control treatment. While not being bound by theory, it is believed that anti-DDR1 therapy disrupts collagen barriers surrounding solid tumors, allowing increased T cell infiltration, and that the addition of an anti-PD-1 therapeutic increases T cell activation and infiltration to further enhance the anti-tumor effect (FIG. 8).

### Example 5—In Vivo Syngeneic Mouse Tumor Model Selection: Identification of DDR1 Dependent Mouse Models of Immune Exclusion

The objective of these studies was to identify DDR1 dependent syngeneic mouse models of immune exclusion for evaluating novel DDR1-targeted therapies. The selection criteria used for identifying immune exclusion mouse tumor models included: (i) expression of DDR1; (ii) presence of a functioning immune system; (iii) immunohistochemical (IHC) evidence that immune cells are present in the periphery of the tumor (i.e., excluded); and (iv) resistance to checkpoint inhibition therapy or other treatment to enable investigation of combination strategies (e.g., checkpoint inhibitor (CPI); chemotherapy, radiation therapy (RT), etc.)

Eight different syngeneic mouse tumor models that express variable levels of DDR1, having variable levels of resistance to anti-PD1, and known to be immune models were chosen for the selection of a DDR-1 dependent mouse model of immune exclusion. (Table 14). Clean cell lines (i.e., free of mycoplasma and mouse-pathogen contamination) were used for the studies described herein. The knockout cell lines used were generated using transient expression of CRISPR/Cas9 system (i.e., no stable Cas9 expression) to ensure that no non-mouse protein is expressed in any of the cell lines. Further, rather than isolating a single cell clone, DDR1-negative (DDR1 KO) cell pools were sorted using flow cytometry to dilute out any potential off-target effect. The sorted cells were expanded and implanted into wild-type immunocompetent mice of the same, syngeneic strain.

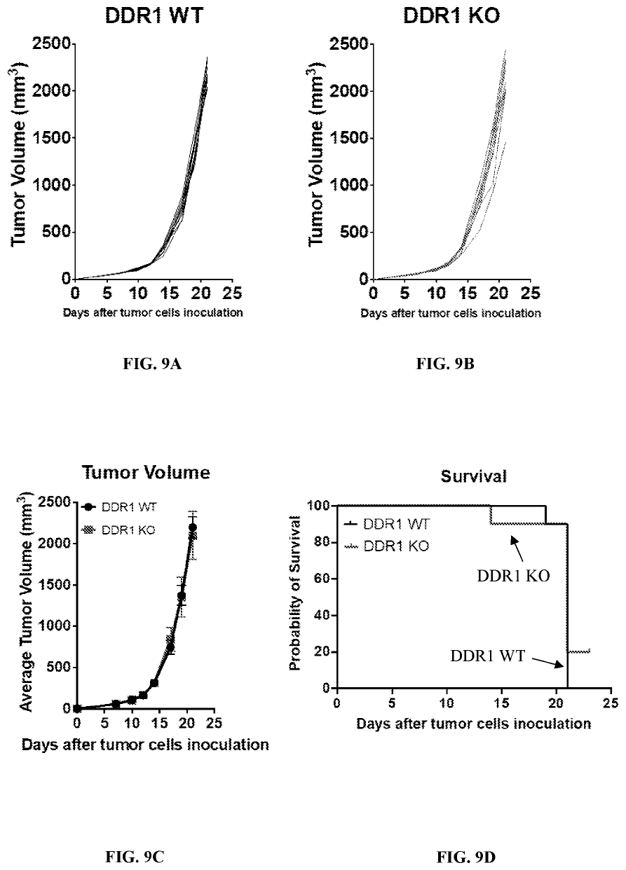

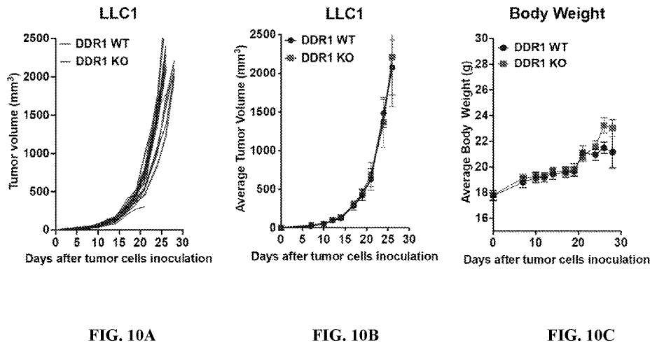

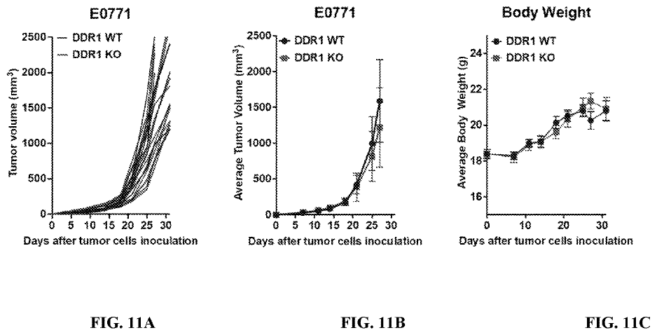

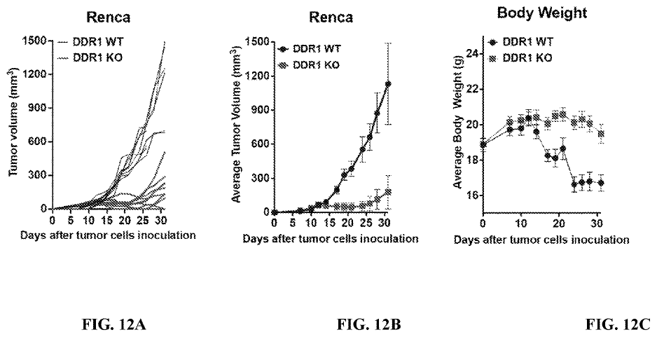

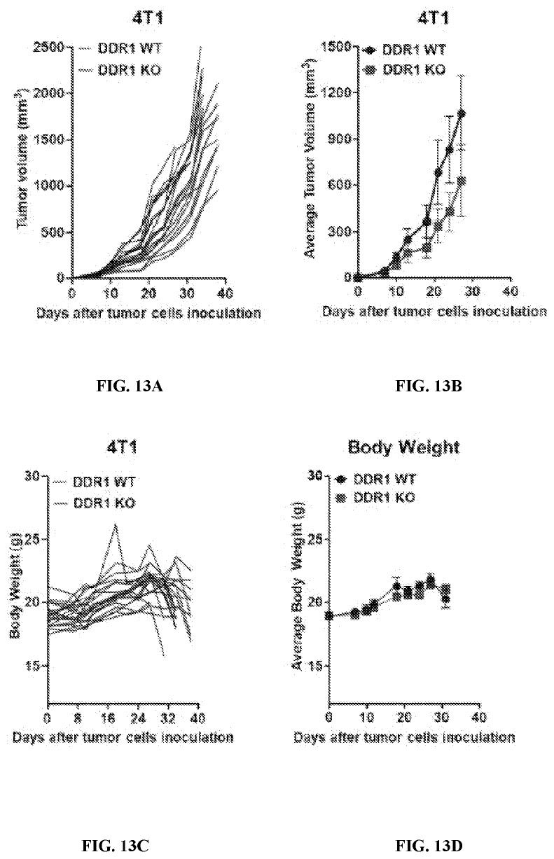

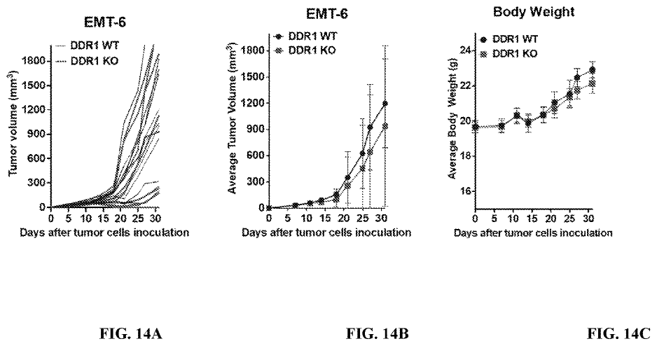

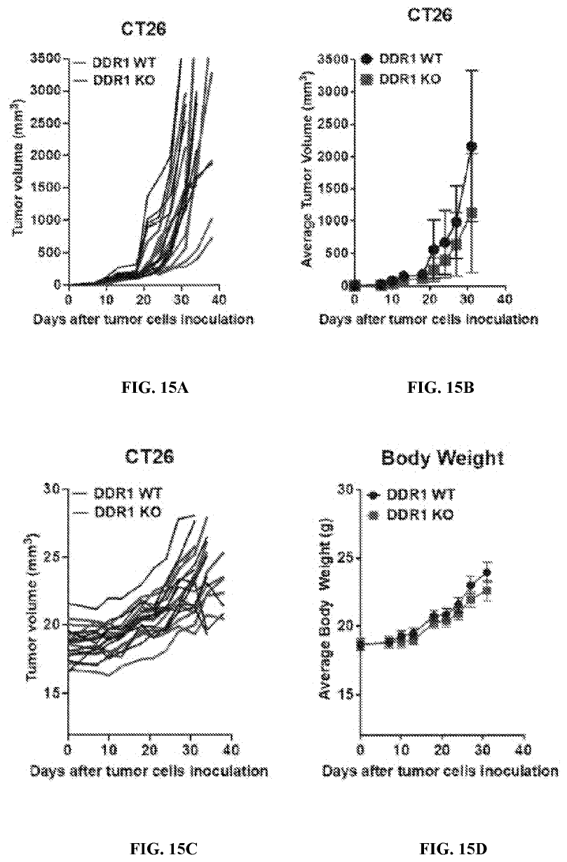

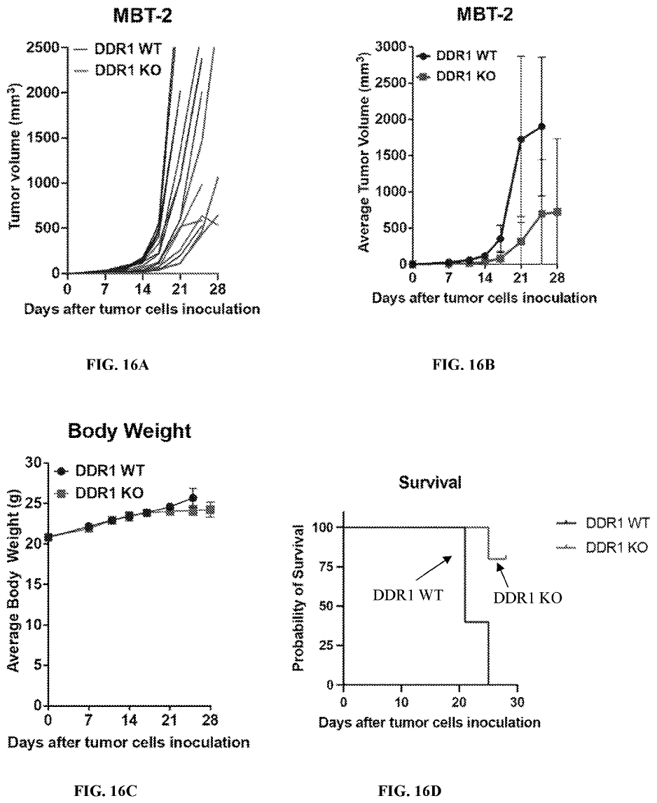

The tumor growth data observed confirmed the anti-tumor effect with DDR1 KO. (e.g., FIG. 12A through FIG. 16D). Interestingly, the anti-tumor effect was observed predominantly in mouse models of the BALB/c strain (FIG. 12A-15D), and C3H/HeN strain (FIG. 16A-16D), and to a lesser extent in C57BL/6 mouse tumor models (FIG. 9A-11C). It was observed that the Renca (renal cancer) and the MBT-2 mouse models demonstrated strong anti-tumor effects. (FIG. 12A-12C; FIG. 16A-16D). The observed anti-tumor effects are summarized in Table 14 below.

TABLE 14
Summary of antitumor effects observed in mouse tumor models.

| | | | Anti-tumor effect |
|---|---|---|---|
| Model | Tumor Type | Host Strain | observed with DDR1−/− |
| B16F10 | Melanoma | C57BL/6 | − |
| LLC1 | Lewis lung | C57BL/6 | − |
| E0771 | Breast | C57BL/6 | − |
| Renca | Renal | Balb/c | +++ |
| 4T1 | Breast | Balb/c | ++ |
| EMT6 | Breast | Balb/c | ++ |
| CT26 | Colon | Balb/c | + |
| MBT-2 | Urothelial | C3H/HeN | +++ |

### Example 6—Tumor Kinetics Study with Murine Colorectal Carcinoma Tumor Model-CT26 (Wild Type DDR1 (WT) and DDR1 Knockout (KO)) Cell Lines in Balb/c Mice

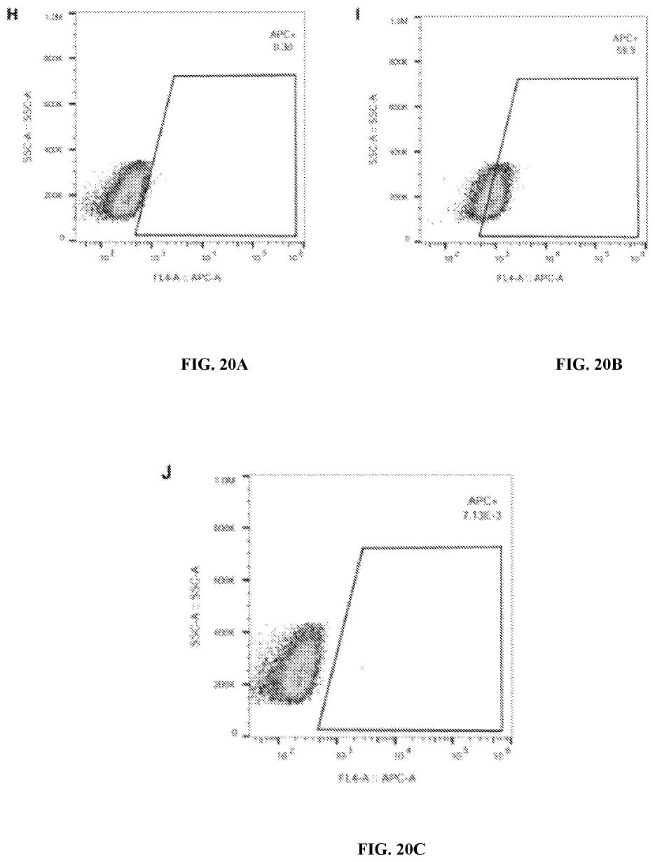

A CRISPR-Cas9 system was used to perturb DDR1 expression in the CT26 murine colorectal cells referred to in Example 5. Following that, isolated CT26 cells with decreased surface DDR1 levels (DDR1r/DDR1 KO) compared to WT control were sorted using flow cytometry (FIG. 20A-20C). DDR1r/DDR1 KO and control cells (DDR1 WT) were injected into the flanks of BALB/c mice (n=10 for each condition) to form tumors. Tumor volumes at 31 days (where all mice were still in the study) showed a significant reduction in tumor size in the DDRIr/DDR1 KO condition. These results demonstrated that DDR1 plays a functional role in restraining tumor growth and progression.

### Methods and Results:

DDR1 CRISPR knockout: DDR1 was knocked out in the mouse colon carcinoma cell line CT26 (ATCC, CRL-2638) by using two DDR1 sgRNAs and Cas9-RFP (IDT, Cat #10008163). Both DDR1 sgRNAs target DDR1 extracellular domain-encoding sequences.

sgRNA sequences are as follows: sgRNAI: TCCATCTCCACGTAGCCCGTGGG (IDT, Design ID: Mm.Cas9.DDR1.1.AC); [SEQ ID NO: 13]; sgRNA2: ACTTACGATG-GATATACTGCTGG (Design ID: Mm.Cas9.DDR1.1.AD). [SEQ ID NO: 14]. The RNP complex with Cas9-RFP and sgRNA was generated in vitro following the manufacturer's instructions. The RNP complex was electroporated into CT26 cells with a Lonza Nucleofector™ System.

FACS sorting: A CT26 DDR1 knockout cell pool was isolated by cell sorting (Sony SH800S). Briefly, CT26 cells were collected 72 hours after electroporation and cell surface DDR1 expression was detected by anti-mouse DDR1 antibody (Sun et al. Nature, 2021 November; 599 (7886): 673-678; antibody #33). A DDR1-negative population was sorted out for continuous culture for one week. Sorted and expanded cells were collected for a second round of sorting to generate a >99% DDR1-negative population. This CT26 cell pool tested negative for mycoplasma and murine pathogens (Mouse Essential CLEAR panel, Charles River Research Animal Diagnostic Services).

Tumor mouse model: Female BALB/c mice (strain BALB/cAnNCrl) (n=20; age=6-8 weeks) obtained from Charles River Laboratories were used for the present study. The body weight of the mice at the time of inoculation was about 17-25 g.

CT26 tumor cell lines were used for the study and are described below in Table 15 and Table 16.

TABLE 15
CT26 (WT) [WT: Wild type DDR1]

| Tumor cell line: | CT26 (WT) |
|---|---|
| Source: | Parthenon Therapeutics |
| Passage # (user stock): | P0 |
| Passage # (inoculated): | P2 |
| Preparation media (+10% FBS): | DMEM |
| Adherent or suspension: | Adherent |
| Initial viable cell conc. (10⁶ cells/mL): | 1.95e6 |
| Volume of initial viable cell conc. used (mL): | 27 |
| Final conc. (10⁶ cells/mL): | 3.0 |
| Matrigel Yes/No? | No |
| Volume of final cell conc. (mL): | 17.55 |
| # Cells (×10⁶) injected per mouse: | 0.3 |
| Volume injected per mouse (mL): | 0.1 |
| Implant route: | SC |
| Implant site: | Right lower flank |
| Cell Viability before Inoculation (%): | 94.6 |
| Cell Viability after Inoculation (%): | 89.1 |

TABLE 16
CT26 (KO) [KO: DDR1 Knockout]

| Tumor cell line: | CT26 (KO) |
|---|---|
| Source: | Parthenon Therapeutics |
| Passage # (user stock): | P1 |
| Passage # (inoculated): | P3 |
| Preparation media (+10% FBS): | DMEM |
| Adherent or suspension: | Adherent |
| Initial viable cell conc. (10⁶ cells/mL): | 1.22e6 |
| Volume of initial viable cell conc. used (mL): | 27 |
| Final conc. (10⁶ cells/mL): | 3.0 |
| Matrigel Yes/No? | No |
| Volume of final cell conc. (mL): | 19.98 |
| # Cells (×10⁶) injected per mouse: | 0.3 |
| Volume injected per mouse (mL): | 0.1 |
| Implant route: | SC |
| Implant site: | Right lower flank |
| Cell Viability before Inoculation (%): | 87.9 |
| Cell Viability after Inoculation (%): | 94.2 |

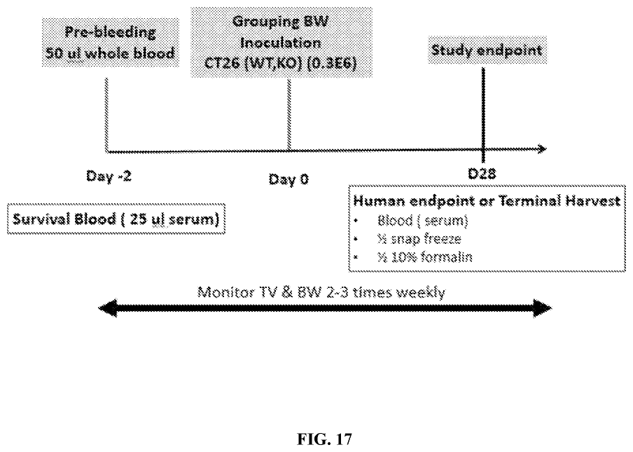

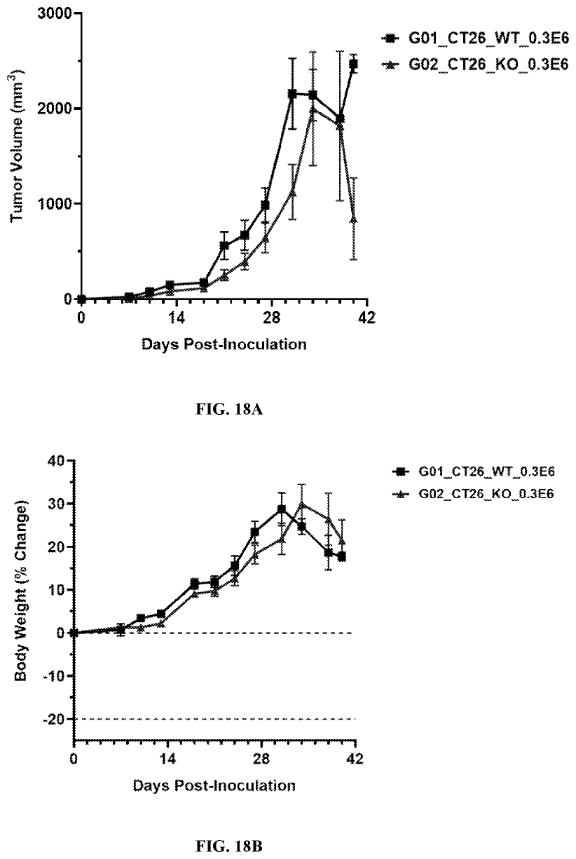

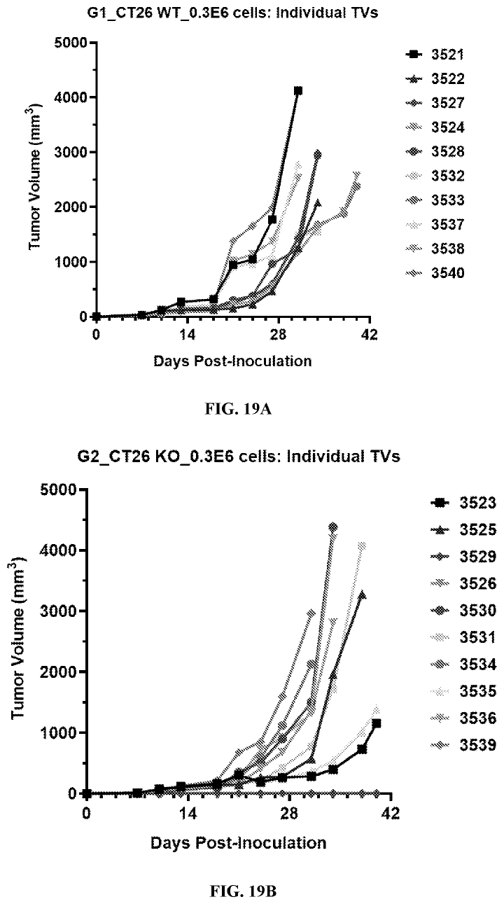

FIG. 17 shows the design of the tumor kinetics study with CT26 (wild type DDR1 (WT) and DDR1 knock out (KO)) cell lines in Balb/c Mice. Tumor volumes (TV) (mm³) and body weight (BW) were monitored 2-3 times weekly over the entire period of study, and the changes plotted as shown in FIG. 18A and FIG. 18B. Mice were removed from the average if its tumor volume reached >2,000 mm³ tumor volume or upon animal death, whatever occurred earlier. This led to a reduction in average tumor volume in later timepoints. Individual tumor volumes are shown in FIG. 19A and FIG. 19B.

On Day 31 of the study, all the mice were alive and TV (mean±SEM) of animals in CT26 WT (Group 1 (G1)) was 2156.77+371.38 mm³ and CT26 KO (Group 2 (G2)) was 1126.50+290.45 mm³.

Body weight (BW) changes were not different between the groups throughout the study. There were no unexpected deaths or clinical observations during the study except for mouse #3532 in G1, which was euthanized due to morbidity.

Survival and terminal blood (serum) were collected two days prior to the beginning of the study (Day 2) and at the study endpoint (Day 28), respectively. Further, at the study endpoint (Day 28), one half (½) of the tumors were put into 10% formalin and the other half (½) of the tumors were snap frozen with liquid nitrogen. (FIG. 17).

### INCORPORATION BY REFERENCE

All patent and non-patent literature references cited above are incorporated herein by reference in their entirety.

What is claimed is:

1. A method of reducing immune exclusion of a tumor in a subject in need thereof, the method comprising administering to the subject an anti-DDR1 antibody that specifically binds to human DDR1 and an immune checkpoint inhibitor, wherein the anti-DDR1 antibody is administered at a dose of 5 mg to 2000 mg.

2. A method of reducing tumor burden in a subject in need thereof, the method comprising administering to the subject an anti-DDR1 antibody that specifically binds to human DDR1 and an immune checkpoint inhibitor, wherein the anti-DDR1 antibody is administered at a dose of 5 mg to 2000 mg.

3. The method of claim 1 or 2, wherein the anti-DDR1 antibody is administered at a dose of about 8 mg to about 1600 mg.

4. The method of claim 1 or 2, wherein the anti-DDR1 antibody is administered at a dose of about 8 mg to about 800 mg.

5. The method of any one of claims 1-4, wherein the anti-DDR1 antibody is administered at a dose of about 8 mg, about 24 mg, about 80 mg, about 240 mg, about 400 mg, about 800 mg, or about 1600 mg.

6. The method of any one of claims 1-5, wherein the anti-DDR1 antibody is administered at a dose of 8 mg, 24 mg, 80 mg, 240 mg, 400 mg, 800 mg, or 1600 mg.

7. The method of any one of claims 1-6, wherein the anti-DDR1 antibody is administered intravenously.

8. The method of any one of claims 1-7, wherein the anti-DDR1 antibody is administered via intravenous infusion over 60 minutes.

9. The method of any one of claims 1-7, wherein the anti-DDR1 antibody is administered via intravenous infusion over 30 minutes.

10. The method of any one of claims 1-9, wherein the anti-DDR1 antibody is administered once weekly.

11. The method of any one of claims 1-9, wherein the anti-DDR1 antibody is administered once every 2 weeks.

12. The method of any one of claims 1-9, wherein the anti-DDR1 antibody is administered once every 3 weeks.

13. The method of any one of claims 1-9, wherein the anti-DDR1 antibody is administered once every 4 weeks.

14. The method of any one of claims 1-9, wherein the anti-DDR1 antibody is administered once every 8 weeks.

15. The method of any one of claims 1-9, wherein the anti-DDR1 antibody is administered intravenously at a dose of 8 mg once every 3 weeks.

16. The method of any one of claims 1-9, wherein the anti-DDR1 antibody is administered intravenously at a dose of 24 mg once every 3 weeks.

17. The method of any one of claims 1-9, wherein the anti-DDR1 antibody is administered intravenously at a dose of 80 mg once every 3 weeks.

18. The method of any one of claims 1-9, wherein the anti-DDR1 antibody is administered intravenously at a dose of 240 mg once every 3 weeks.

19. The method of any one of claims 1-9, wherein the anti-DDR1 antibody is administered intravenously at a dose of 400 mg once every 3 weeks.

20. The method of any one of claims 1-9, wherein the anti-DDR1 antibody is administered intravenously at a dose of 800 mg once every 3 weeks.

21. The method of any one of claims 1-9, wherein the anti-DDR1 antibody is administered intravenously at a dose of 1600 mg once every 3 weeks.

22. The method of any one of claims 1-21, wherein the immune checkpoint inhibitor comprises a PD-1 or PD-L1 antagonist.

23. The method of any one of claims 1-22, wherein the PD-1 or PD-L1 antagonist is administered at a dose of 100 mg to 2000 mg.

24. The method of any one of claims 1-22, wherein the PD-1 or PD-L1 antagonist is administered at a dose of 200 mg, 240 mg, 350 mg, 360 mg, 400 mg, 480 mg, 500 mg, 840 mg, 1000 mg, 1200 mg, 1500 mg, or 1680 mg.

25. The method of any one of claims 1-22, wherein the PD-1 or PD-L1 antagonist is administered at a dose of 0.5 mg/kg to 30 mg/kg.

26. The method of any one of claims 1-22, wherein the PD-1 or PD-L1 antagonist is administered at a dose of 1 mg/kg, 2 mg/kg, 3 mg/kg, 10 mg/kg, or 20 mg/kg.

27. The method of any one of claims 1-26, wherein the PD-1 or PD-L1 antagonist is administered intravenously.

28. The method of any one of claims 1-27, wherein the PD-1 or PD-L1 antagonist is administered once weekly.

29. The method of any one of claims 1-27, wherein the PD-1 or PD-L1 antagonist is administered once every 2 weeks.

30. The method of any one of claims 1-27, wherein the PD-1 or PD-L1 antagonist is administered once every 3 weeks.

31. The method of any one of claims 1-27, wherein the PD-1 or PD-L1 antagonist is administered once every 4 weeks.

32. The method of any one of claims 1-27, wherein the PD-1 or PD-L1 antagonist is administered once every 6 weeks.

33. The method of any one of claims 1-27, wherein the PD-1 or PD-L1 antagonist is administered once every 8 weeks.

34. The method of any one of the preceding claims, wherein the subject has cancer.

35. The method of claim 34, wherein the administration of the anti-DDR1 antibody and the PD-1 or PD-L1 antagonist treats the cancer in the subject.

36. The method of claim 34, wherein the PD-1 antagonist is an anti-PD-1 antibody that specifically binds to human PD-1.

37. The method of claim 34, wherein the PD-L1 antagonist is an anti-PD-L1 antibody that specifically binds to human PD-L1.

38. The method of claim 36, wherein the anti-PD-1 antibody is pembrolizumab, nivolumab, dostarlimab, cemiplimab, sintilimab, penpulimab, tislelizumab, toripalimab, or retifanlimab.

39. The method of claim 37, wherein the anti-PD-L1 antibody is avelumab, atezolizumab, or durvalumab.

40. The method of any one of claims 1-36, wherein the PD-1 antagonist is pembrolizumab.

41. The method of claim 40, wherein pembrolizumab is administered at a dose of 400 mg once every 6 weeks.

42. The method of claim 40, wherein pembrolizumab is administered at a dose of 200 mg once every 3 weeks.

43. The method of claim 40, wherein pembrolizumab is administered at a dose of 2 mg/kg once every 3 weeks.

44. The method of any one of claims 1-36, wherein the PD-1 antagonist is nivolumab.

45. The method of claim 44, wherein nivolumab is administered at a dose of 240 mg once every 2 weeks.

46. The method of claim 44, wherein nivolumab is administered at a dose of 360 mg once every 3 weeks.

47. The method of claim 44, wherein nivolumab is administered at a dose of 480 mg once every 4 weeks.

48. The method of claim 44, wherein nivolumab is administered at a dose of 3 mg/kg once every 2 weeks.

49. The method of claim 44, wherein nivolumab is administered at a dose of 3 mg/kg once every 3 weeks.

50. The method of any one of claims 1-36, wherein the PD-1 antagonist is cemiplimab.

51. The method of claim 50, wherein cemiplimab is administered at a dose of 350 mg once every 3 weeks.

52. The method of any one of claims 1-36, wherein the PD-1 antagonist is dostarlimab.

53. The method of claim 52, wherein dostarlimab is administered at a dose of 500 mg once every 3 weeks.

54. The method of claim 52, wherein dostarlimab is administered at a dose of 1000 mg once every 6 weeks.

55. The method of any one of claim 1-35, or 37, wherein the PD-L1 antagonist is atezolizumab.

56. The method of claim 55, wherein atezolizumab is administered at a dose of 840 mg once every 2 weeks.

57. The method of claim 55, wherein atezolizumab is administered at a dose of 1200 mg once every 3 weeks.

58. The method of claim 55, wherein atezolizumab is administered at a dose of 1680 mg once every 4 weeks.

59. The method of any one of claim 1-35, or 37, wherein the PD-L1 antagonist is durvalumab.

60. The method of claim 59, wherein durvalumab is administered at a dose of 10 mg/kg once every 2 weeks.

61. The method of claim 59, wherein durvalumab is administered at a dose of 20 mg/kg once every 3 weeks.

62. The method of claim 59, wherein durvalumab is administered at a dose of 1500 mg once every 3 weeks.

63. The method of any one of the preceding claims, wherein the cancer expresses DDR1.

64. The method of any one of the preceding claims, wherein the cancer is a solid cancer.

65. The method of any one of the preceding claims, wherein the cancer is a locally advanced or metastatic solid cancer.

66. The method of any one of the preceding claims, wherein the cancer is unresectable.

67. The method of any one of the preceding claims, wherein the cancer is refractory to immunotherapy.

68. The method of claim 67, wherein the immunotherapy is an antagonist anti-PD-1 antibody, an antagonist anti-PD-L1 antibody, an antagonist anti-PD-L2 antibody, an antagonist anti-PD-1/anti-PD-L1 antibody bispecific antibody, an antagonist anti-CTLA-4 antibody, an antagonist anti-BTLA antibody, an antagonist anti-TREMR antibody, an antagonist anti-TIGIT antibody, an antagonist anti-VISTA antibody, an antagonist anti-TIM-3 antibody, an antagonist anti-LAG-3 antibody, an antagonist anti-CEACAM1 antibody, an agonist anti-GITR antibody, an agonist anti-OX40 antibody, and an agonist anti-CD137 antibody, an agonist anti-DR3 antibody, an agonist anti-TNFSF14 antibody, an agonist anti-CD27 antibody, an agonist anti-ICOS antibody, or an agonist anti-CD28 antibody.

69. The method of claim 67, wherein the immunotherapy is an antagonist anti-PD-L1 antibody.

70. The method of any one of the preceding claims, wherein the cancer is not sarcoma, hepatocellular carcinoma, or glioma.

71. The method of any one of the preceding claims, wherein the cancer is pancreatic cancer, lung cancer, small cell lung cancer, non-small cell lung cancer, colorectal cancer, head and neck cancer, stomach (gastric) cancer, ovarian cancer, breast cancer, kidney cancer, prostate cancer, cervical cancer, brain cancer, skin cancer, melanoma, cholangiocarcinoma, or bone cancer.

72. The method of any one of the preceding claims, wherein the cancer is colorectal cancer, ovarian cancer, or non-small cell lung cancer.

73. The method of any one of the preceding claims, wherein the subject is not a candidate for standard of care treatment.

74. The method of any one of the preceding claims, wherein the cancer is refractory to a standard of care treatment.

75. The method of claim 74, wherein the standard of care treatment is chemotherapy or radiation.

76. The method of any one of claims 1-75, wherein administration of the anti-DDR1 antibody and the PD-1 or PD-L1 antagonist reduces tumor size in the subject.

77. The method of any one of the preceding claims, wherein before administration of the anti-DDR1 antibody and the PD-1 or PD-L1 antagonist the subject:
a) has confirmed metastatic or advanced, unresectable cancer, measurable disease per Response Evaluation Criteria in Solid Tumors (RECIST) v1.1;
b) has a pathologically documented advanced, unresectable, or metastatic cancer that is refractory to or intolerable to standard treatment known to confer benefit or for which no standard treatment is available;
c) has an Eastern Cooperative Oncology Group performance status (PS) 0-1;
d) has a predicted life expectancy of ≥3 months;
e) has one or more of:
$$i ) calculated creatinine clearance ( CrCL ) \geq 50 mL / min by Crockcroft - Gault fomulation calculation ;$$
$$ii ) total bilirubin \leq 1.5 ;$$
$$iii ) AST and ALT \leq 2.5 \times ULN ;$$
$$iv ) hemoglobin \geq 9. g / dL ;$$
$$v ) platelets \geq 100 \times {10}^{9} cells / L ; or$$
$$vi ) absolute neutrophil count \geq 1.5 \times {10}^{9} cells / L ;$$
f) has a Corrected QT interval (QTc)≤470 ms (as calculated by the Fridericia correction formula); and/or
g) is not receiving other cancer therapy.

78. The method of any one of the preceding claims, wherein before administration of the anti-DDR1 antibody and the PD-1 or PD-L1 antagonist the subject:
a) has not received prior treatment with systemic agents comprising radio-immunoconjugates, antibody-drug conjugates, immune/cytokines or monoclonal antibodies within 28 days or five half-lives of the drug, whichever is shorter;
b) does not have ongoing toxicity from prior therapy;
c) has not undergone a major surgery<3 months prior to administration of the anti-DDR1 antibody;
d) has not received radiation therapy<28 days prior to administration of the anti-DDR1 antibody;
e) has not undergone organ transplantation, allogeneic stem-cell transplantation, or autologous stem-cell transplantation;
f) has not received a diagnosis of primary or acquired immunodeficiency;
g) has not received treatment with systemic steroids or any other form of immunosuppressive therapy within 14 days prior to administration of the anti-DDR1 antibody;
h) does not have central nervous system (CNS) tumor involvement not definitively treated with surgery or radiation that is active;
i) does not have active autoimmune disease requiring immunosuppressive therapy or a history of such disease;
j) does not have clinical symptoms of CNS metastases within 28 days prior to administration of the anti-DDR1 antibody; and/or
k) does not have leptomeningeal carcinomatosis.

79. The method of any one of the preceding claims, wherein the anti-DDR1 antibody comprises: a heavy chain variable region (VH) comprising the CDRH1, CDRH2, and CDRH3 amino acid sequences of the VH amino acid sequence set forth in SEQ ID NO: 7, or a variant thereof comprising 1-5 amino acid changes in any one of the CDRH1, CDRH2, or CDRH3 amino acid sequences; and/or a light chain variable region (VL) comprising the CDRL1, CDRL2, and CDRL3 amino acid sequences of the VL amino acid sequence set forth in SEQ ID NO: 8 or 9, or a variant thereof comprising 1-5 amino acid changes in any one of the CDRL1, CDRL2, or CDRL3 amino acid sequences.

80. The method of claim 79, wherein:
(a) the VH comprises the CDRH1, CDRH2, and CDRH3 amino acid sequences, respectively, of:
SEQ ID NO: 1, or a variant thereof comprising 1-5 amino acid changes,
SEQ ID NO: 2, or a variant thereof comprising 1-5 amino acid changes, and
SEQ ID NO: 3, or a variant thereof comprising 1-5 amino acid changes; and/or
(b) the VL comprises the CDRL1, CDRL2, and CDRL3 amino acid sequences, respectively, of:
SEQ ID NO: 4, or a variant thereof comprising 1-5 amino acid changes,
SEQ ID NO: 5, or a variant thereof comprising 1-5 amino acid changes, and
SEQ ID NO: 6, or a variant thereof comprising 1-5 amino acid changes.

81. The method of claim 79 or 80, wherein the anti-DDR1 antibody that specifically binds to human DDR1 comprises the CDRH1, CDRH2, CDRH3, CDRL1, CDRL2, and CDRL3 amino acid sequences set forth in SEQ ID NOs: 1, 2, 3, 4, 5, and 6, respectively.

82. The method of any one of claims 79-81, wherein the anti-DDR1 antibody that specifically binds to human DDR1 comprises: a VH comprising an amino acid sequence which is at least 75%, 80%, 85%, 90%, 95%, 96%, 97%, 98%, 99%, or 100% identical to the amino acid sequence set forth in SEQ ID NO: 7; and/or a VL comprising an amino acid sequence which is at least 75%, 80%, 85%, 90%, 95%, 96%, 97%, 98%, 99%, or 100% identical to the amino acid sequence set forth in SEQ ID NO: 8 or 9.

83. The method of claim 82, wherein the anti-DDR1 antibody comprises a VH comprising the amino acid sequence set forth in SEQ ID NO: 7 and a VL comprising the amino acid sequence set forth in SEQ ID NO: 8.

84. The method of claim 82, wherein the anti-DDR1 antibody comprises a VH comprising the amino acid sequence set forth in SEQ ID NO: 7 and a VL comprising the amino acid sequence set forth in SEQ ID NO: 9.

85. The method of any one of claims 79-84, wherein the anti-DDR1 antibody comprises a heavy chain comprising an amino acid sequence which is at least 75%, 80%, 85%, 90%, 95%, 96%, 97%, 98%, 99%, or 100% identical to the amino acid sequence set forth in SEQ ID NO: 10 or 11 and/or a light chain comprising an amino acid sequence which is at least 75%, 80%, 85%, 90%, 95%, 96%, 97%, 98%, 99%, or 100% identical to the amino acid sequence set forth in SEQ ID NO: 12.

86. The method of any one of the preceding claims, wherein the anti-DDR1 antibody comprises a heavy chain comprising the amino acid sequence set forth in SEQ ID NO: 10 and a light chain comprising the amino acid sequence set forth in SEQ ID NO: 12.

87. The method of any one of claims 1-85, wherein the anti-DDR1 antibody comprises a heavy chain comprising the amino acid sequence set forth in SEQ ID NO: 10, without the terminal lysine, and a light chain comprising the amino acid sequence set forth in SEQ ID NO: 12.

88. The method of any one of claims 1-85, wherein the anti-DDR1 antibody comprises a heavy chain comprising the amino acid sequence set forth in SEQ ID NO: 11 and a light chain comprising the amino acid sequence set forth in SEQ ID NO: 12.

89. The method of any one of claims 1-85, wherein the anti-DDR1 antibody comprises a heavy chain comprising the amino acid sequence set forth in SEQ ID NO: 11, without the terminal lysine, and a light chain comprising the amino acid sequence set forth in SEQ ID NO: 12.

90. The method of any one of the preceding claims, wherein the anti-DDR1 is administered to the subject before the PD-1 or PD-L1 antagonist.

91. The method of any one of the preceding claims, wherein the anti-DDR1 antibody is administered to the subject after the PD-1 or PD-L1 antagonist.

92. The method of any one of the preceding claims, wherein the anti-DDR1 antibody is administered to the subject at the same time as the PD-1 or PD-L1 antagonist.

93. The method of any one of the preceding claims, wherein administration of the antibody and the immune checkpoint inhibitor prevents further growth of tumor size in the subject.

94. The method of any one of the preceding claims, wherein administration of the antibody and the immune checkpoint inhibitor achieves at least stable disease in the subject.

95. An anti-DDR1 antibody and a PD-1 or PD-L1 antagonist for use in the treatment of cancer, wherein the treatment is performed according to the method of any one of the previous claims.

96. An anti-DDR1 antibody and a PD-1 or PD-L1 antagonist for use in the manufacture of a medicament for the treatment of cancer, wherein the treatment is performed according to the method of any one of the previous claims.

97. Use of an anti-DDR1 antibody and a PD-1 or PD-L1 antagonist for the treatment of cancer, wherein the treatment is performed according to the method of any one of the previous claims.

98. A therapeutic combination comprising an antibody that specifically binds to human DDR1 and a PD-1 or PD-L1 antagonist.

99. A combination comprising an anti-DDR1 antibody and a PD-1 or PD-L1 antagonist for use in the treatment of cancer, wherein the treatment is performed according to the method of any one of the previous claims.
